(19) United States
(12) Patent Application Publication
(10) Pub. No.: US 2026/0000872 A1
(43) Pub. Date: Jan. 1, 2026

(54) INDWELLING CANNULA

(71) Applicant: EBNET MEDICAL GMBH, Schwerin (DE)

(72) Inventors: Jens EBNET, Schwerin (DE); WERNER SCHWARTZ, Rulpolding (DE)

(21) Appl. No.: 18/568,035

(22) PCT Filed: Jun. 15, 2022
(86) PCT No.: PCT/EP2022/066380

§ 371 Date: Feb. 28, 2025

(30) Foreign Application Priority Data
Jun. 18, 2021 (DE) 10 2021 115 847.8
Aug. 23, 2021 (DE) 10 2021 121 790.3
Nov. 9, 2021 (DE) 20 2021 106 110.3

(51) Int. Cl. A61M 25/06; A61M 25/00; A61M 25/01; A61M 25/09

(52) U.S. Cl. CPC A61M 25/0606; A61M 25/005; A61M 25/0113; A61M 25/09; A61M 2025/0059

(57) ABSTRACT

The invention relates to an indwelling cannula (1) for puncturing a hollow body by means of a puncture needle, wherein the indwelling cannula (1) has a catheter (2) with a vasiform catheter tube (22), in which the puncture needle can be guided longitudinally displaceably. The indwelling cannula (1) has a manually actuable forward feed mechanism (5) by which a relative movement can be generated between the catheter tube (22) and the puncture needle as a result of manual actuation, by which a puncture tip (31), protruding from the catheter tube (22) at the near-patient end, of the puncture needle can be received in the catheter tube (22). Preferably, at least one operating element of the forward feed mechanism (5) is configured as a pivotably mounted lever (53) with at least one lever arm.

The invention relates to an indwelling cannula for puncturing a hollow body by means of a puncture needle, wherein the indwelling cannula has at least one catheter with a vasiform catheter tube, in which the puncture needle can be guided longitudinally displaceably, wherein the catheter is adapted to be pushed over at least a part of the length of the catheter tube through the opening created by means of the puncture needle through the tunic of the hollow body to be punctured after puncture of a tunic of the hollow body to be punctured has been carried out, and to remain there over a period of time. The invention will be described with the aid of various embodiments, which may be combined with one another in any desired way.

### 1ST EMBODIMENT

### Indwelling Cannula

The invention relates to an indwelling cannula for puncturing a hollow body by means of a puncture needle, wherein the indwelling cannula has at least one catheter with a vasiform catheter tube, in which the puncture needle can be guided longitudinally displaceably, wherein the catheter is adapted to be pushed over at least a part of the length of the catheter tube through the opening created by means of the puncture needle through the tunic of the hollow body to be punctured after puncture of a tunic of the hollow body to be punctured has been carried out, and to remain there over a period of time.

Such an indwelling cannula may, for example, be configured as an indwelling venous cannula. The hollow body to be punctured may be a hollow body of a living being or a hollow body of an object. If the indwelling cannula relates to the medical field, the hollow body may for example be a blood vessel, for example a vein or artery.

The terms “puncturing” and “puncture” are in this case to be understood in the medical sense. “Puncturing” refers to the entry of a puncture needle into the hollow body, in such a way that the puncture needle penetrates through the tunic of the hollow body to be punctured.

Those parts which are arranged at the remote (distal) end of the indwelling cannula, from the viewpoint of the user, and therefore in the vicinity of the punctured hollow body, are regarded as a section of parts of the indwelling cannula near to the hollow body. Accordingly, sections remote from the hollow body are arranged at the proximal end of the indwelling cannula from the user's viewpoint, i.e. further away from the punctured hollow body. In connection with medical applications of the indwelling cannula, the terms “near-vein” or “near-patient” or “user-remote” are also used synonymously with the term “near to the hollow body”, and “vein-remote” and “patient-remote” and “near-user” are also used synonymously for the term “remote from the hollow body”.

Near-vein components tend to be located inside the patient, and vein-remote components tend to be arranged outside the patient. This need not necessarily always apply, but is meant to further clarify the terms. The additional elements are generally self-evident and the context is essential.

A pierce-resistant indwelling venous cannula is disclosed by PCT/EP2019/057097.

An improved puncture system in the sense of a general puncture system is intended to be provided. In principle, with the improved indwelling cannula it is likewise possible advantageously to puncture all body cavities and interstitial body spaces and all anatomical and morbid structures which are intended to be punctured, and to install a catheter in them.

Components of the improved indwelling cannula may in principle also be combined with all known puncture and catheter systems or be used as independent products.

Although the term “indwelling cannula” will be adopted below, it has a wider meaning in the sense of a general puncture system with which not only veins may be punctured. In what follows, the terms “vein” and “veins” thus include in principle all blood vessels and very generally all body cavities and interstitial body spaces and all anatomical and morbid structures which are intended to be punctured and provided with a catheter.

Thus, in addition, by way of example the trachea, pleural space, abdomen, stomach, intestine, renal pelvis, urinary bladder, structures of the central and peripheral nervous system, cerebrospinal fluid space and bone may be punctured with the indwelling cannula. In addition, morbid structures such as abscesses in and on the patient may be punctured. Advantageously, arterial blood vessels may also be punctured.

In what follows, the term “patient” includes all living beings of all age groups and genders. Applications in technical fields and in and on all objects and structures, for example in and on reservoirs, containers, cavities, extensible materials and in or on pump, hose, pipe and port systems, are likewise explicitly possible.

When the word “or” is used in what follows, possible alternatives are indicated but combinations of the features or configurations separated by “or” are also fundamentally explicitly possible.

All components described may be employed singly or multiply on an indwelling cannula or also, independently of the latter, on/in other products or entirely independently. Various features of different components are also freely combinable, and features of particular components may also be used in other components without this being explicitly mentioned. All components and features may fundamentally be employed inside and outside a patient.

Indwelling cannulas, and in particular indwelling venous cannulas, are special medical devices insofar as they must have particular diameter and length proportions in order to be able to be applied in human and animal patients at the usual venous access sites. For this purpose, a certain flexibility or resilience of the catheter tube is also necessary. Such indwelling cannulas are therefore dissimilar to catheter systems for other applications, for example a urinary catheter, because entirely different requirements apply in those cases.

The object of the invention is to provide an indwelling cannula which is easier and safer to use.

This object is achieved by an indwelling cannula as claimed in claim 1. Advantageous developments are specified in the dependent claims.

According to one advantageous configuration of the invention, the indwelling cannula has a grip region which is adapted in such a way that the indwelling cannula is gripped in the grip region during manipulation by the user. The grip region of the indwelling cannula may in this case be a region in which there are no other functions or functional elements of the indwelling cannula, but which is provided only for manual gripping of the indwelling cannula. This has the advantage that the user intuitively grasps the indwelling cannula at the proper site, so that unnecessary touching at other sites is avoided, for example at sites of the indwelling cannula which must necessarily remain sterile. In this way, unnecessary contaminations of particular sites of the indwelling cannula can be avoided. For example, the grip region may be formed on the housing of the catheter, for example shaped integrally with the housing. The grip region may, for example, be produced by a corresponding configuration of regions of the outer surface of the housing.

For example, ribs, pimples or other surface structuring may be present on the grip region. The grip region may likewise at least partially consist of a material which increases frictional resistance or be coated with at least one such material. Inadvertent slipping of the fingers of the user is thereby prevented. The grip region may also consist for example of an elastomer, for example rubber or silicone, or of any other material which is softer than the rest of the material of the housing of the indwelling cannula.

The grip region may also consist of at least one antimicrobially active material or be coated with at least one such material. It may likewise have particularly hydrophobic properties. Properties in the sense of or similar to a lotus effect may also be envisioned.

The grip region may also be configured at least partly in a different color than the other components of the indwelling venous cannula, for example in the color green. Regions of the indwelling venous cannula which should in no case be touched may be configured at least partly in the color red. The color “yellow” may also be envisioned at least partially in regions which should not be touched but the touching of which is nevertheless possible in special situations. This is meant to remind the user of the colors of a traffic light. In particular, strongly luminous or fluorescent colors may however also be envisioned.

In a further advantageous version, the entire indwelling cannula is configured to be transparent so that flows of fluid/blood can be recognized directly. The puncture needle may also be configured to be transparent, if the material properties allow this.

Special patterns, for example cross-like or arrow-like patterns, are likewise possible in the grip region. This region may also have numbering or in general labelling. It is likewise conceivable for the grip region to be initially covered by an adhering structure, for example a film, which may be removed after the puncture process.

The features explained for the grip region may also be implemented individually or in combination on one or more of the grip faces explained below.

According to one advantageous configuration of the invention, it is provided that the indwelling cannula has a manually actuable forward feed mechanism by which a relative movement can be generated between the catheter tube and the puncture needle as a result of manual actuation, by which a puncture tip, protruding from the catheter tube at the near-patient end, of the puncture needle can be received in the catheter tube. Even more advantages are thereby achieved when using the indwelling cannula. In conventional indwelling cannulas, the installation on the patient, for example on a vein, is carried out in such a way that the vein is initially punctured with a puncture tip, jutting out beyond the catheter tube, of the puncture needle. After the vein has been punctured, the catheter tube is pushed forward manually beyond the puncture needle, which may in this case also already be withdrawn at least partially into the catheter tube. These steps are left to the discretion and skill of the user. For inexperienced persons or in particularly difficult situations, errors may in this case occur, for example by pushing the catheter tube forward too far or not pushing the catheter tube forward far enough, so that a part of the puncture tip still protrudes from the catheter tube.

By the manually actuable forward feed mechanism present in the indwelling cannula according to the invention, defined handling by the user and correspondingly a defined relative movement between the catheter tube and the puncture needle, which is predefined by the design of the indwelling cannula, are ensured. In particular, by the design of the forward feed mechanism it is possible to ensure that the puncture tip is fully received in the catheter tube and therefore can no longer lead to injuries during the further insertion of the indwelling cannula into the hollow body.

By the puncture needle located inside the catheter tube, the relatively resilient or flexible catheter tube is also supported, i.e. braced.

Materials for the puncture needle may be metal materials, in particular stainless steel or fundamentally metal alloys. The puncture needle may, however, also consist of a plastic. It may also be formed in lightweight construction or from at least one material which is resorbable/readily soluble in blood solutions and infusion solutions, for example a sugar or a salt, for example in the region of the puncture tip. Magnesium may also be used. The puncture needle may likewise advantageously be developed with at least one sensor, for example in the region of the puncture tip.

By the design of the forward feed mechanism, it is in this case likewise possible at the same time to ensure that the puncture needle is not withdrawn too far into the catheter tube, so that bracing of the catheter tube can still take place substantially over its entire length. This is not the case with conventional indwelling cannulas because of the fact that how far the puncture needle is withdrawn is up to the preference of the user.

According to one advantageous configuration of the invention, it is provided that, by the forward feed mechanism, the catheter tube can be pushed beyond the puncture tip of the puncture needle and/or the puncture tip of the puncture needle can be retracted into the catheter tube. This allows a large degree of flexibility in the design of the manually actuable forward feed mechanism. Selectively, therefore, it may be possible to push the catheter tube forward in relation to the puncture needle or to retract the puncture needle in relation to the catheter tube, or a combination thereof may be possible.

According to one advantageous configuration of the invention, it is provided that one, several or all of the operating elements of the manually actuable forward feed mechanism are arranged in the grip region and/or form parts of the grip region. This has the advantage that the indwelling cannula can be handled particularly easily by the user, and is suitable in particular for one-handed operation throughout the entire process of installing the indwelling cannula on the patient.

The manually actuable forward feed mechanism may wholly or partially, at least with its predominant elements, for example the manual operating elements, be part of the needle device of the indwelling cannula. This has the advantage that the needle device and therefore also the puncture needle in the case of successful puncture of the vein, often recognizable by a return blood flow into the puncture needle, can be held by the user in a stable, ideally static position, and the distal end of the catheter tube can initially be pushed forward precisely beyond the puncture tip into the vein before the catheter tube braced by the puncture needle is then pushed forward further into the vein. In this case, the puncture needle is configured together with the aforementioned parts of the manually actuable forward feed mechanism as a module which can be removed as a whole from the module connected to the catheter tube, in particular the housing, after correct placement of the catheter tube on the patient, i.e. in the hollow body to be punctured.

The catheter tube can be applied accurately via the puncture needle held stably in position. The puncture needle may in this case be extracted further back out of the catheter tube at any time.

It is advantageous for the user to hold the puncture needle/needle device firmly with their fingers all of the time.

A withdrawal of the puncture needle controlled individually by the user is by construction not prevented at any time with the indwelling venous cannula according to the invention. This has the advantage that the user always keeps control over the withdrawal of the puncture needle, which allows an optimal combination of technical and user control.

According to one advantageous configuration of the invention, it is provided that the relative movement between the catheter tube and the puncture needle, which can be generated by manual actuation of the forward feed mechanism, is limited to a maximum value. This has the advantage that operation is made particularly easy for the user, and the design of the forward feed mechanism ensures that errors are avoided when pushing the catheter tube forward relative to the puncture needle. The limitation of the relative movement to the maximum value may, for example, be achieved by the nature of the force transmission from the operating elements of the forward feed mechanism to the module connected to the catheter tube. For example, the forward feed mechanism may have at least one mechanical stop by which the relative movement that can be generated by the manual actuation between the catheter tube and the puncture needle is limited to the maximum value. The limitation to the maximum value may, however, also be achieved without such a mechanical stop.

According to one advantageous configuration of the invention, it is provided that the maximum value of the relative movement is at least as great as the length of the puncture tip. This ensures that a sufficient relative movement for fully receiving the sharp-edged puncture tip in the catheter tube is guaranteed by the forward feed mechanism.

According to one advantageous configuration of the invention, it is provided that the maximum value of the relative movement is less than two times the length of the puncture tip. This ensures that the puncture needle is not withdrawn unnecessarily far into the catheter tube and sufficient support in the sense of bracing of the catheter tube by the puncture needle is maintained. According to a further advantageous configuration of the invention, it is provided that the maximum value of the relative movement is less than three times the length of the puncture tip.

A puncture tip is in this case intended to mean that near-patient region of the puncture needle which is pointed, for example by a bevel or by conical shaping. The length of the puncture tip is therefore the extent of the puncture tip in the longitudinal direction of the puncture needle.

The puncture tip may in this case be configured so that it can penetrate intact patient skin without substantial pushing forward by the user. In a further version, because of it shaping, it does not penetrate the patient skin without considerable pushing forward, or even kinks before penetrating the skin. The puncture tip is thus configured less sharply/more bluntly. The invention may thereby advantageously be used directly in very injured/soft anatomical regions to which, for example, an access through the skin has already been made surgically. For example, the invention may be used in the region of the central nervous system, particularly in the region of the brain.

If the puncture tip has the aforementioned bevel, various puncture techniques are possible. In the bevel-up technique, the puncture tip is inserted through the skin by the bevel, including the inner opening of the puncture needle that is present therein, pointing upward, i.e. being turned away from the skin of the patient. In the bevel-down technique, by contrast, the puncture tip is twisted by 180° in relation to the catheter tube, so that the bevel, including the inner opening of the puncture needle that is present therein, points downward, i.e. is turned toward the skin of the patient.

The indwelling cannula may, for example, be configured in such a way that the puncture needle, or at least the puncture tip, may at any time be twisted in relation to the catheter tube about the longitudinal axis. The user may then selectively employ the bevel-up technique or the bevel-down technique. The indwelling cannula may also be configured in such a way that in at least one particular longitudinal displacement position, for example when the puncture tip protrudes from the catheter tube at the near-patient end, the puncture needle or at least the puncture tip cannot be twisted in relation to the catheter tube, or can be twisted only with increased force exertion. For example, the module having the puncture needle may be coupled in this longitudinal displacement position with a form fit to the module having the catheter tube and the twistability of the puncture needle in relation to the catheter tube may thereby be prevented. In this case, for example, the indwelling cannula may be configured in such a way that the bevel, including the inner opening of the puncture needle that is present therein, points downward from the start, i.e. is turned toward the skin of the patient or a lower side of the indwelling cannula, on which at least one fixing wing of the indwelling cannula is arranged.

According to a further advantageous configuration of the invention, it is provided that the puncture tip can be twisted/rotated about its own longitudinal axis and/or the longitudinal axis of the puncture needle, and can in this case preferentially be set to a circular measurement of 45°, 90°, 135°, 180°, 225°, 270°, 315° or 360° (angle specifications in degrees with respect to a circular measurement of 360 degrees (360°)), all intermediate stages are conceivable in respect of the circular measurement. The bevel of the puncture tip in this case already by design no longer necessarily points upward before use of the indwelling cannula according to the invention and is thus no longer necessarily turned away from the skin of the patient. This may advantageously allow gentler puncture techniques since, inter alia, puncture/entry angles may be varied. The puncture needle or the module having the puncture needle may in this case have holding/path limiting elements (in what follows, only “holding elements”) by which the twistability/rotatability of the puncture tip/puncture needle is restricted or negated at least in particular longitudinal displacement positions of the puncture tip and/or puncture needle and/or needle device in relation to the catheter tube. In one advantageous configuration of the invention, the twistability/rotatability just described of the puncture tip/puncture needle is fully negated when the puncture tip projects at least partially beyond the catheter tube distally (in the direction of the patient). The holding elements may in this case, for example, be configured as latching elements, notches, furrows or projections. These may interact with holding elements that are located on the catheter tube or on further components of the indwelling cannula according to the invention, for example by latching, wedging or distorting. All holding elements may at least partially consist of a material which increases the frictional resistance or be coated with at least one such material. The aforementioned angle specifications may be made discernible for the user on a component of the indwelling cannula, so that the current circular measurement at which the puncture tip and/or puncture needle is set can be read off easily. Here, for example, circular or semicircular markings in a different line width and, for example, also markings in the traffic light colors “green”, “yellow” and “red” may be envisioned. It is also conceivable that initial puncture and further pushing of the puncture tip and/or puncture needle forward into the tissue to be punctured is carried out in such a way that the puncture tip and/or puncture needle is sequentially set to a different circular measurement, i.e. twisted/rotated about the longitudinal axis of the puncture tip/puncture needle in the chronological sequence of the puncture process.

According to one advantageous configuration of the invention, it is provided that the forward feed mechanism can be fixed by means of locking in the manually actuated position in which the puncture tip is received in the catheter tube. This has the advantage that accidental withdrawal of the puncture tip back out of the catheter tube is avoided by the design of the forward feed mechanism. Once the forward feed mechanism has been manually actuated and the puncture tip is received in the catheter tube, the locking prevents the puncture tip from being inadvertently moved back out of the catheter tube.

According to one advantageous configuration of the invention, it is provided that, in the manually actuated position of the forward feed mechanism, in which the puncture tip is received in the catheter tube, at least one operating element of the forward feed mechanism can be fixed by means of the locking and/or at least a part of the housing of the catheter can be fixed on the needle device. This allows simple implementation of the locking of the forward feed mechanism in the manually actuated position. The operating element of the forward feed mechanism may, for example, be fixable by means of the locking on a further operating element assigned as a mating piece, or on another part of the needle device.

According to one advantageous configuration of the invention, it is provided that at least one operating element of the forward feed mechanism is configured as a pivotably mounted lever with at least one lever arm. In this way, a reliably working robust forward feed mechanism is provided, which can be operated easily and intuitively by the user. The lever may for example have a force arm and a load arm, whereby the lever is pivotably mounted at a position between the force arm and the load arm by means of a bearing element. The load arm is in this case the lever arm which exerts a force onto a constituent part connected to the catheter tube, for example onto the housing of the indwelling cannula. The force arm is the lever arm on which the user applies the actuation force, for example on a grip face of the force arm. In this case, the lengths of the force arm and load arm may be equal or different. For example, the load arm may be longer than the force arm.

As an actuation element for generating the pushing of the catheter tube forward in relation to the puncture needle, there may for example also be a twistably mounted wheel which transmits a forward feed force during the rotation of the wheel onto the catheter tube or a constituent part connected thereto by force-fit coupling and/or friction. A force-fit coupling between the twistably mounted wheel and the catheter tube or the constituent part connected thereto may, for example, be provided by a link/pin guide or by gearing. Such a twistably mounted wheel will be regarded in what follows as a special case of a lever.

The required relative movement between the catheter tube and the puncture needle, by which the puncture tip is ultimately received in the catheter tube, may be generated by a manually applied actuation force alone. The relative movement may also be additionally assisted by a spring force, i.e. by the force of a pretensioned spring by which a relative movement triggered manually by the user is fully or partially continued by the spring force. For example, the indwelling cannula may have as a manual operating element merely a trigger element for triggering the relative movement, the further performance of the relative movement then being carried out by spring force.

According to one advantageous configuration of the invention, it is provided that the forward feed mechanism has at least two grip faces as operating elements, on each of which at least one finger of the user can be placed, the at least two grip faces being arranged turned away from one another. This has the advantage that the user can grip the indwelling cannula very easily and hold and operate it on the same grip faces throughout the entire application process on the patient. In this way, one-handed operation is possible without the user having to change the grip position during the process of installing on the patient (no regripping necessary). Because the two grip faces are arranged turned away from one another, the user can grip these grip faces from two sides and thereby hold the indwelling cannula securely.

According to one advantageous configuration of the invention, it is provided that at least one of the grip faces is arranged on a lever arm of a pivotably mounted lever of the forward feed mechanism. Secure operation of the forward feed mechanism is thereby ensured. The user can place the fingers required for the operation intuitively at the correct site.

According to one advantageous configuration of the invention, it is provided that the lever can be twisted about a rotation axis, the grip face arranged on the lever extending from a position which is further away than the rotation axis from the puncture needle to a position which is closer to the puncture needle than the rotation axis, at least in the unactuated state of the forward feed mechanism. This has the advantage that inadvertent misactuations of the forward feed mechanism during the insertion of the puncture tip of the puncture needle into the hollow body are avoided. By the corresponding placement of the grip face relative to the rotation axis, a sufficient torque is not yet transmitted onto the lever during the puncture process, so that the forward feed mechanism is not inadvertently activated too early. In general, it may be stated that the rotation axis should not be arranged too close to the puncture needle, in order also to allow a sufficient actuation path for the pushing of the catheter tube forward relative to the puncture needle. The grip face arranged on the lever may, for example, be predominantly located on the force arm of the lever, although it may also extend at least over a subregion of the load arm.

According to one advantageous configuration of the invention, it is provided that a forward feed force for carrying out the pushing of the catheter tube forward in relation to the puncture needle may be transmitted by the lever onto the catheter tube, or a constituent part connected thereto, at a site which is arranged closer to the distal end of the catheter tube than the rotation axis D about which the lever is pivotably mounted.

According to one advantageous configuration of the invention, it is provided that the puncture needle is configured together with the manually actuable forward feed mechanism as a module, the constituent parts of which are firmly connected to one another, for example in such a way that they cannot be released from one another without a tool. In particular, the pivotably mounted lever may be configured as a fixed constituent part of this module, which contains the forward feed mechanism and the puncture needle. If the puncture needle is intended to be removed after carrying out a puncture of a vessel, in this way the entire module is always removed, i.e. the puncture needle with the forward feed mechanism including the lever.

According to one advantageous configuration of the invention, it is provided that the lever can be pivoted from an initial position, in which the tip of the puncture needle protrudes fully from the catheter tube, continuously into an end position in which the puncture needle is fully received in the catheter tube. This allows very delicate handling of the indwelling venous cannula during application on a patient.

According to one advantageous configuration of the invention, it is provided that the forward feed mechanism has at least one securing element by which an actuation of the forward feed mechanism is prevented until the securing element is released. For example, the securing element may be configured in such a way that the pivotable lever cannot yet be pivoted initially, i.e. in the secured state. Only by deliberate actuation of an unsecuring element is the securing negated and the lever can be pivoted. Securing against inadvertent pushing of the catheter tube forward in relation to the puncture needle can thereby be provided. For example, the unsecuring of the securing element may be carried out in such a way that a particular part of the forward feed mechanism must first be actuated in a particular direction, particularly in a direction different to that in which the lever is to be actuated for the pivoting. For example, in order to unsecure the securing element the lever may first need to be pressed down in the direction toward the puncture needle, or parts of the lever may first need to be pressed together laterally in order to negate the securing.

According to one advantageous configuration of the invention, it is provided that the indwelling cannula, in particular its catheter tube, has one or more markings by which the relative movement generated as a result of the manual actuation of the forward feed mechanism between the catheter tube and the puncture needle can be displayed. In this way, the user can easily monitor visually whether a sufficient relevant movement takes place between the catheter tube and the puncture needle. In particular, the current setting that has been generated by the forward feed mechanism may be read visually.

According to one advantageous configuration of the invention, it is provided that the forward feed mechanism has at least one intermediate setting which can be haptically detected by the user, for example in the form of a latch setting, between its end settings. In this way, one or more intermediate positions of the relative movement of the puncture needle generated in relation to the catheter tube may be predefined by design and actively adjustable by the user. By the haptic feedback, the user can easily recognize the intermediate setting that they have reached, for example by an increased resistance occurring in the respective intermediate setting during the actuation of the forward feed mechanism. In this way, the rigidity of the catheter tube may be varied and adapted to individual anatomical situations. For instance, in the case of fragile blood vessels it may be advantageous if the catheter tube is already configured more flexibly during insertion into the blood vessel, particularly in the near-patient region, in order to ensure gentler insertion into the blood vessel.

One property of conventional indwelling venous cannulas is the design-related unrestricted longitudinally displaceability of the inner-lying puncture needle in relation to the outer-lying catheter tube. After successful puncture, the puncture needle can thereby be withdrawn/extracted from the catheter tube according to its function in the direction of the user, and therefore removed. The somewhat flexible catheter tube, however, often requires bracing after successful puncture when pushing forward into the vein, so that it can be pushed forward securely into the vein. This bracing is carried out by the inner-lying puncture needle. In conventional indwelling venous cannulas, however, the extent of the withdrawal of the puncture needle is uncertain. In the event of insufficient withdrawal, the tip of the puncture needle may still be free at the near-vein end of the catheter tube and therefore inadvertently damage anatomical structures. In the event of excessive withdrawal, the catheter tube can no longer be braced sufficiently when pushing forward into the vein. However, the puncture needle may also be pushed forward in the direction of the vein again after withdrawal. This may lead to the catheter tube being damaged by the tip of the puncture needle and therefore also, for example, to parts of the catheter tube being sheared off into the vein.

With the indwelling cannula according to the invention, after successful vein puncture, the puncture needle is withdrawn over a defined distance in the longitudinal direction into the catheter tube. This has the advantage that the user can be certain that the tip of the puncture needle no longer projects beyond the catheter tube in the direction of the vein in the longitudinal direction. In this state, the tip is completely enclosed by the outer-lying catheter tube and inadvertent injury to anatomical structures is thus avoided.

With the indwelling cannula according to the invention, however, the puncture needle is also prevented from being withdrawn too far away from the vein into the catheter tube during the puncture process and the bracing of the tube is therefore prevented from being insufficient. The puncture needle thus braces the catheter tube over an accurately defined distance and precisely stabilizes it when pushing forward into the vein. The distance may also be displayed by markings on the indwelling cannula. The user may also vary it precisely by corresponding ribs/indentations, into which for example a grip piece may be latched, being applied on the indwelling cannula.

Advantageously, it is also possible to predefine a maximum value of the relative movement between the catheter tube and the puncture needle, which can be overcome only with particular exertion by the user in order to be able to remove the puncture needle. This prevents the puncture needle from inadvertently slipping completely out of the indwelling cannula. What has been described above is relevant in particular when the catheter tube is configured very flexibly and/or for example also has spiral or undulating structure, as may be the case with the indwelling cannula according to the invention.

This may also be particularly relevant when the indwelling cannula is configured very flexibly, as just described, only at the transition site between the catheter tube and/or only in a vein-remote portion, for example at the level of the skin.

There is maximum safety against inadvertent injury to anatomical structures by the tip of the puncture needle particularly when the catheter tube is additionally configured to be piercing-resistant.

A pronounced thermoplasticity of the catheter tube is no longer absolutely necessary since it can be configured very flexibly and the rigidity often required for pushing forward into the vein is ensured primarily by the puncture needle.

It is also conceivable that, for the puncture, the puncture needle first needs to be pushed forward into a position in which the tip of the puncture needle projects beyond the catheter tube in the direction of the vein.

On the concave bulge (see the drawings), the indwelling cannula may also be fixed on the skin by a suture, in particular when the bulge has ribs/indentations/elevations, lateral recesses and/or further fastening elements.

The indwelling cannula according to the invention is also operable with one hand better than conventional indwelling cannulas. Thus, for example, the other hand of the user may be used to stabilize the body site to be punctured and to stretch the skin. This increases the likelihood of successful puncture.

In the indwelling cannula according to the invention, in contrast to conventional indwelling cannulas, there may also be defined and intuitively operable grip faces for the fingers of the user. The indwelling cannula can therefore no longer be inadvertently polluted/contaminated, in particular no longer in the region of the entry site into the skin and/or in the region of the bearing face onto the skin. The region of the entry site into the skin is furthermore particularly protected by design against pollution/contamination. The grip faces themselves are adapted to the anatomy of the user and may also be configured to be pliable/deformable.

In addition, the fixing of the indwelling cannula according to the invention is facilitated since the fixing wings are connected to the other components of the indwelling cannula only via a narrow web, and for example plasters may therefore be applied more easily. In particular, the wings of a plaster and/or plaster strip may be fed through more easily under the indwelling cannula. It is also conceivable that the wings are fitted pivotably on the indwelling cannula. It is also conceivable that the wings are configured to be pliable and/or deformable and can thus also be adapted optimally to unevennesses in the skin region of the patient. It is also possible that the web consists of at least one pliable and/or deformable and/or resilient material. Advantageously, it may also be developed with at least one spiral and/or spring-like structure. Forces/movements which act on loaded components of the indwelling venous cannula may thereby be reduced in their effect on other components of the indwelling venous cannula, for example cushioned, when there is a web as just described between the loaded components and the other components.

Any conventional/commercially available single-lumen or multilumen catheter tube may be used. According to one advantageous configuration of the invention, however, the catheter tube has at least one supporting structure by which the bending moment of resistance and/or the radial moment of resistance of the catheter tube is increased. The supporting structure may be configured in the form of one or more reinforcing elements.

The supporting structure may, for example, be a spiral structure extending over a certain length section of the catheter tube, which may be integrated on the inner side and/or on the outer side of the catheter tube and/or partially or fully into the catheter tube wall. There may also be a plurality of coaxial supporting structures of this type. The spiral structure may also have spring-like properties. Instead of a spiral structure, there may also be individual supporting rings spaced apart from one another, integrated both on the inner side, the outer side and/or wholly or partially into the catheter tube wall. The spiral structure and/or the rings may be formed from a round material or flat material, in particular from a metal material. At least one supporting structure may also be formed by a thin-walled supporting tunic or a supporting braid consisting of fibers interlaced with one another or fibers joined to form a woven or laid fabric. The catheter tube may be configured in a single ply or multiple plies in the radial direction, both from the same and from different materials in the individual radial plies. For example, a supporting structure may be embedded into an inner radial ply which is covered by an outer radial ply of the catheter tube.

For example, polyurethane (PU) or FEP may be envisioned as materials for the catheter tube. Materials for the supporting structure may be metal materials, in particular stainless steel and/or metal alloys, in particular nitinol. Nitinol has the advantage that the desired resistance of the catheter tube against compression or kinking is increased further by the memory effect.

Further materials for the supporting structure may be plastic materials, in particular also fiber-reinforced, for example carbon- or aramid fiber-reinforced plastics.

In the case of a spiral configuration of the supporting structure, there may be one spiral or a plurality of spirals placed in one another. The turns of the spirals may have the same spacing in front of one another over the entire length of the supporting structure, or varying spacings. In one advantageous configuration of the invention, a spiral supporting structure may have a smaller spacing of the turns in front of one another in the region remote from the hollow body than in the region near to the hollow body.

There may also be further structures, which stabilize the supporting structure, between the turns. In one advantageous version, these structures are tensioned when the turns move away from one another, and thus become more rigid. The structures between the turns also ensure that the turns can only be spaced apart limitedly from one another. When the turns move toward one another, the structures between them become less rigid. In this version, the supporting structure has a structure similar to an accordion.

In one advantageous version, the structures between the turns may form the supporting structure at least partially fluid-impermeably with a corresponding material selection.

By such a supporting structure, when using a material that is visible in imaging medical methods, in particular a metal material, the visibility of the catheter in patients by means of imaging medical methods, for example by ultrasound scanning, may be improved. In one advantageous configuration of the invention, the supporting structure or the sequence of a plurality of supporting structures in the longitudinal direction is configured nonuniformly over the longitudinal extent. The nonuniform configuration makes even more accurate position identification of particular regions of the catheter in the patient possible by means of imaging medical methods.

According to one advantageous configuration of the invention, the catheter tube has a multiplicity of lateral passage openings which extend from the inner side to the outer side. The passage openings (side holes) may be present over the entire length of the catheter tube or only over one or more longitudinal sections, for example in a section near to the hollow body. The passage openings may be arranged distributed over the circumference of the catheter tube. The flow of liquids through the catheter tube can be improved by the passage openings in the catheter tube wall.

According to one advantageous configuration of the invention, one, several or all of the passage openings are arranged in the region of the catheter tube not covered by the supporting structure. If the supporting structure has a spiral shape, for example, passage holes may be arranged between the turns of the spiral. In this way, the flow through the passage openings is not impeded by the supporting structure.

It is also conceivable that a spiral supporting structure is not enclosed by and/or coated with a fluid-impermeable material at least over a partial length of the catheter tube. The indwelling cannula may thereby be permeable for liquids in this region even without passage holes. In one advantageous configuration, the turns of the spiral supporting structure have a larger spacing from one another in this region than in the other regions of the catheter tube. This region may, as described above, preferentially lie near to the hollow body.

It is also conceivable that a fluid-impermeable material enclosing and/or coating the spiral supporting structure has gaps and/or interruptions at least over a partial length and therefore ensures fluid permeability of the indwelling cannula in the region of these gaps/interruptions. Side holes are then no longer absolutely necessary.

The indwelling cannula according to the invention has the advantage that the sites just mentioned that are permeable for liquids, for example side holes, may be arranged only in a region of the catheter tube which is braced by the puncture needle and therefore also internally sealed during the puncture process. During the puncture process, no foreign bodies and/or, for example, also detached skin constituents can therefore obstruct the side holes and/or be drawn inadvertently in the latter into more deeply lying anatomical structures.

In the indwelling cannula according to the invention, it is however also advantageously possible for fluid-permeable sites, for example side holes, to be released precisely and selectively by different positions of the puncture needle in the longitudinal direction, when they are located at different positions on or in the catheter tube in the longitudinal direction. Precisely controlled dilution effects may thus be generated, for example in order to inject vein-irritant substances more tolerably.

In a further advantageous version, the aforementioned material may also only partially enclose the spiral supporting structure continuously in the longitudinal direction of the indwelling cannula. For instance, a kind of web which counteracts uncoiling of the supporting structure may be formed. In the case of very tight winding, the supporting structure may be configured to be fluid-impermeable, while in the case of less tight winding it may however also be configured to be fluid-permeable. The tightness of the winding may also determine whether the supporting structure is, for example, configured to be fluid-impermeable but nevertheless permeable for gases/vapors. It is also conceivable for the supporting structure to be permeable only for particular fluids. It is likewise conceivable for the supporting structure to have thermoplastic properties and/or to expand due to body heat.

According to one advantageous configuration of the invention, the catheter has a housing on which the catheter tube is fitted. The catheter tube extends from a side of the housing directed toward the hollow body. According to one advantageous configuration of the invention, in the region in which the catheter tube projects from the housing, i.e. in a tube exit region of the housing, the catheter has a kink protection structure circumferentially enclosing the catheter tube fully or at least partially, by which the risk of the catheter tube kinking in the region of the exit site from the housing is reduced. In this way, undesired kink formation of the catheter tube when manipulating the indwelling cannula can be avoided, particularly in the region of the exit site from the housing. This region is particularly susceptible to kinks since, in known indwelling venous cannulas, an abrupt transition takes place from the relatively robust, inflexible housing into the comparatively thin-walled and correspondingly sensitive catheter tube. By the kink protection structure according to the invention, the hitherto abrupt transition in this region is made gentler. The kink protection structure may, for example, involve a surface rounded in the shape of a trumpet, which circumferentially encloses the catheter tube.

In one advantageous configuration of the invention, the grip region is configured in such a way that it has two grip faces, which are arranged on mutually opposite sides of the constituent part on which the grip region is present. This has the advantage that the indwelling cannula can be gripped well straightforwardly and securely on the grip region with the thumb and another finger, for example the middle finger or the index finger. The grip faces may, in particular, be predominantly planar grip faces or have an at least predominantly planar part. In their planar regions, the grip faces may be arranged parallel to one another. The grip faces may also be configured in the manner of a trough with an at least locally curved (concave) surface. The grip faces may have structuring by which the indwelling cannula can be gripped even better and slipping of the fingers is avoided.

For example, ribs, pimples or other surface structuring may be present on the grip faces. The grip faces may, in particular, be configured in such a way that their dimension in the longitudinal direction of the indwelling cannula is greater, for example at least two times as great, as the dimension in the transverse direction. In this case, a respective grip face may be formed from the material of the housing of the indwelling cannula or from another material, in particular from a material having greater resilience or flexibility. For example, the grip face may be configured as an insert, for example made of an elastomer, for example rubber, or silicone, or any other material softer than the material of the housing of the indwelling cannula. Such an insert may then be fastened in a corresponding indentation of the housing of the indwelling cannula.

According to one advantageous configuration of the invention, the indwelling cannula has at least one finger stop device on the housing, which is arranged on the tube exit region or near to the tube exit region. The finger stop device is therefore spaced apart significantly from the proximal end of the housing. The finger stop device is used to avoid accidental slipping of the fingers of the user, with which the user grasps the indwelling cannula during application on the patient, from the housing in the longitudinal direction of the indwelling cannula, i.e. slipping toward the patient. The finger stop device therefore forms an obstacle for the fingers against slipping from the housing in the direction of the patient. In this way, contaminations of the entry site on the patient and of parts of the indwelling cannula in the region of the entry site are avoided. The finger stop device may, for example, be configured as a circumferential enlargement in relation to neighboring circumferential regions of the housing, for example in the form of a flange, a bead or another thickening. The finger stop device may circumferentially enclose the housing fully or only in subsections, for example only laterally on the right and left of the catheter tube.

The invention will be explained in more detail below with the aid of exemplary embodiments with the use of drawings, in which:

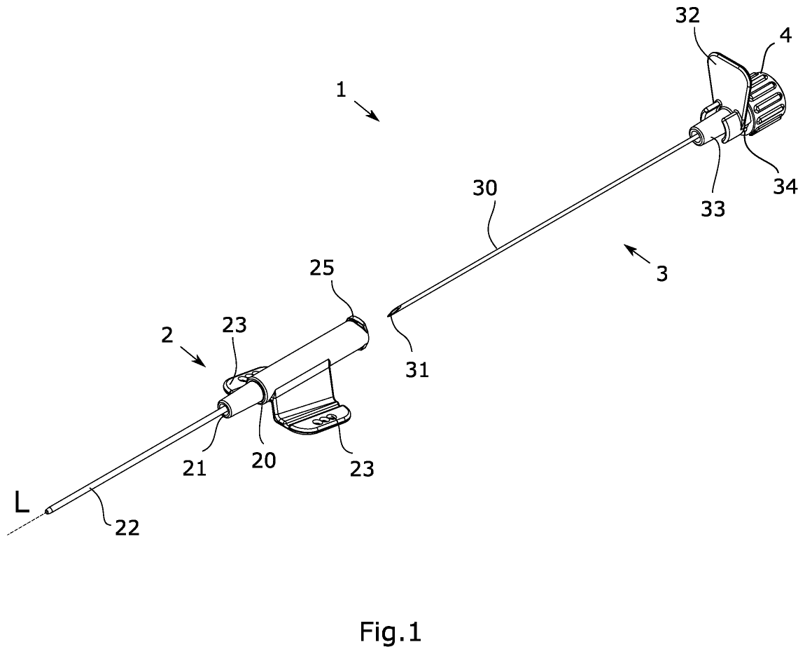

FIG. 1 shows an indwelling cannula in an exploded view,

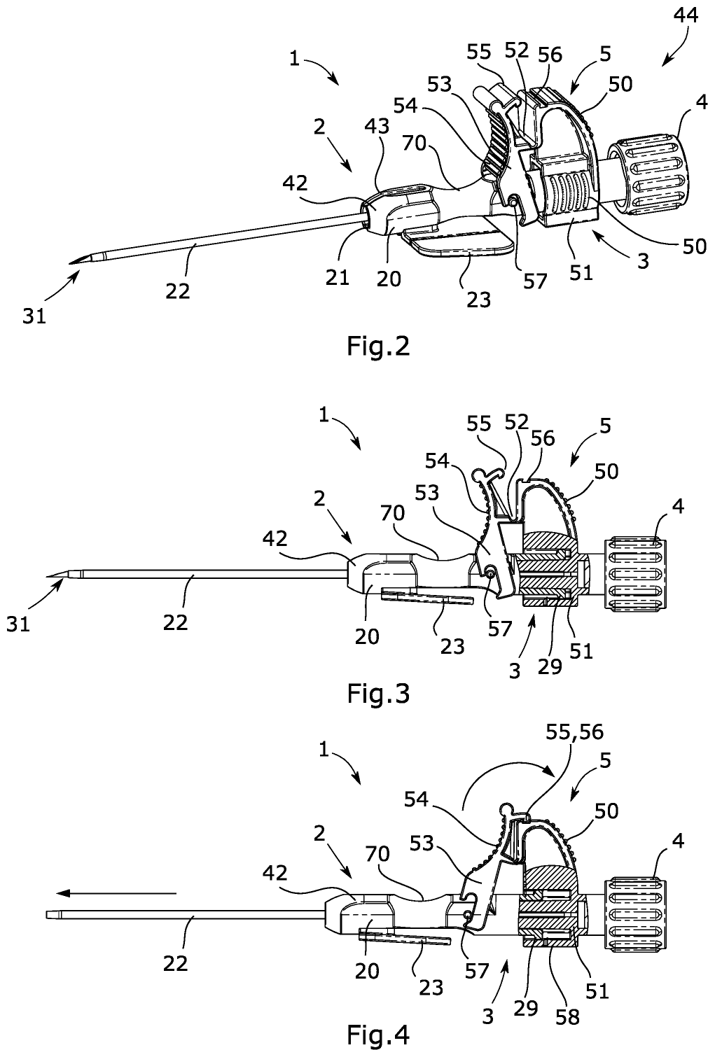

FIG. 2 shows an indwelling cannula in a perspective view,

FIGS. 3, 4 show the indwelling cannula according to FIG. 2 in a partially cutaway side view,

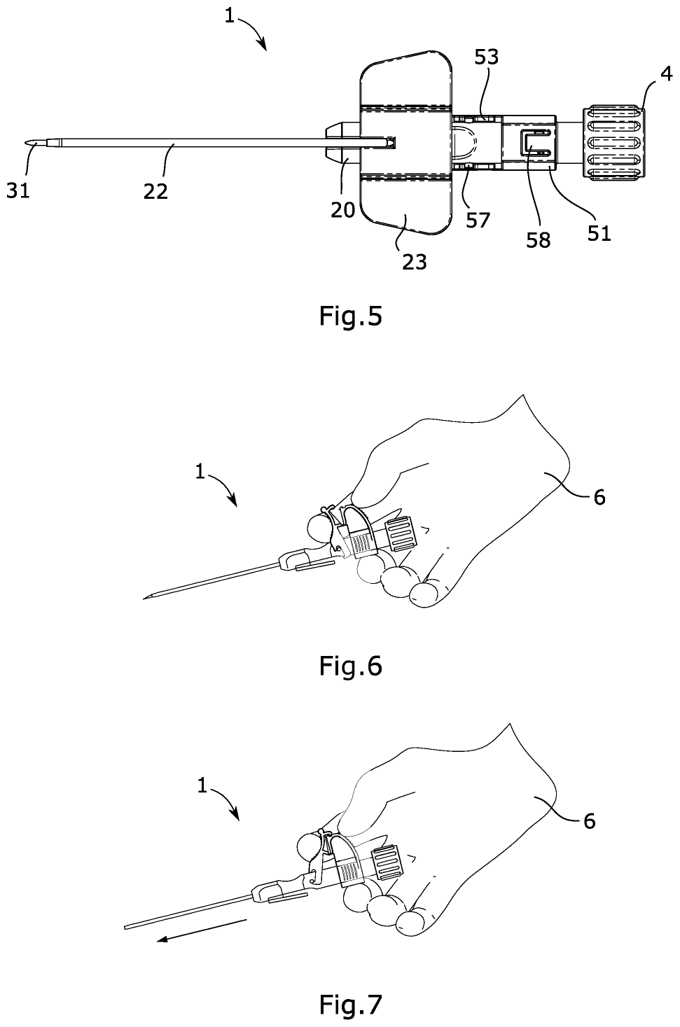

FIG. 5 shows the indwelling cannula according to FIG. 2 in a view from the lower side,

FIGS. 6-8 show the indwelling cannula according to FIG. 2 in various states of use,

FIG. 9 shows an indwelling cannula in a further embodiment in a perspective view,

FIG. 10 shows the indwelling cannula according to FIG. 9 in a side view,

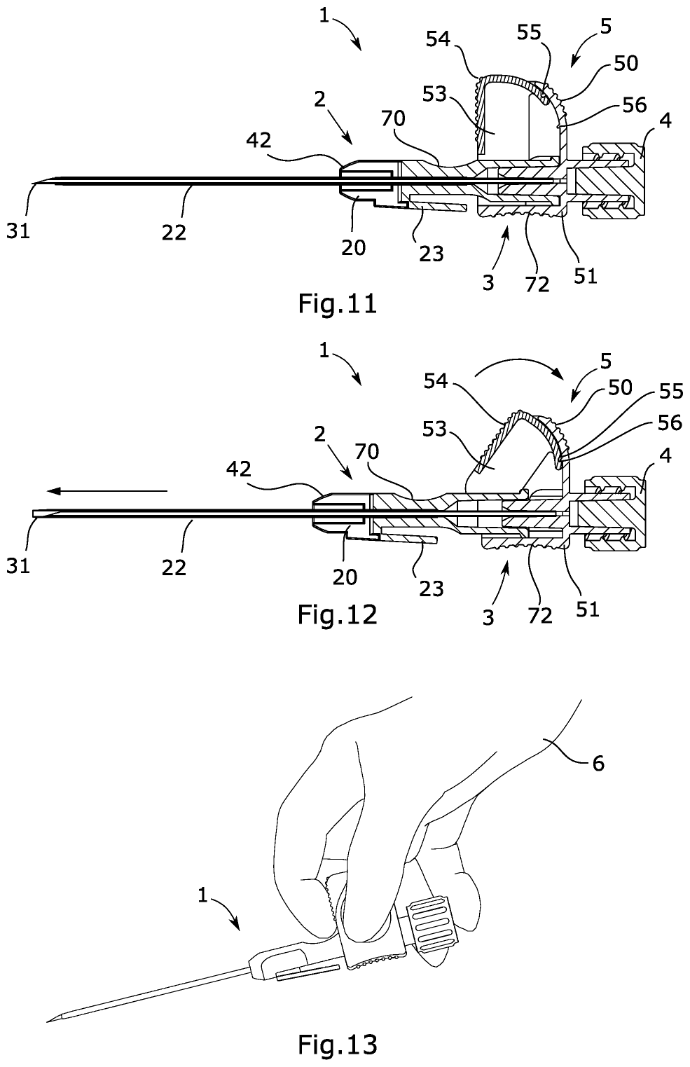

FIGS. 11, 12 show the indwelling cannula according to FIG. 9 in a partially cutaway side view,

FIG. 13 shows the indwelling cannula according to FIG. 9 during use,

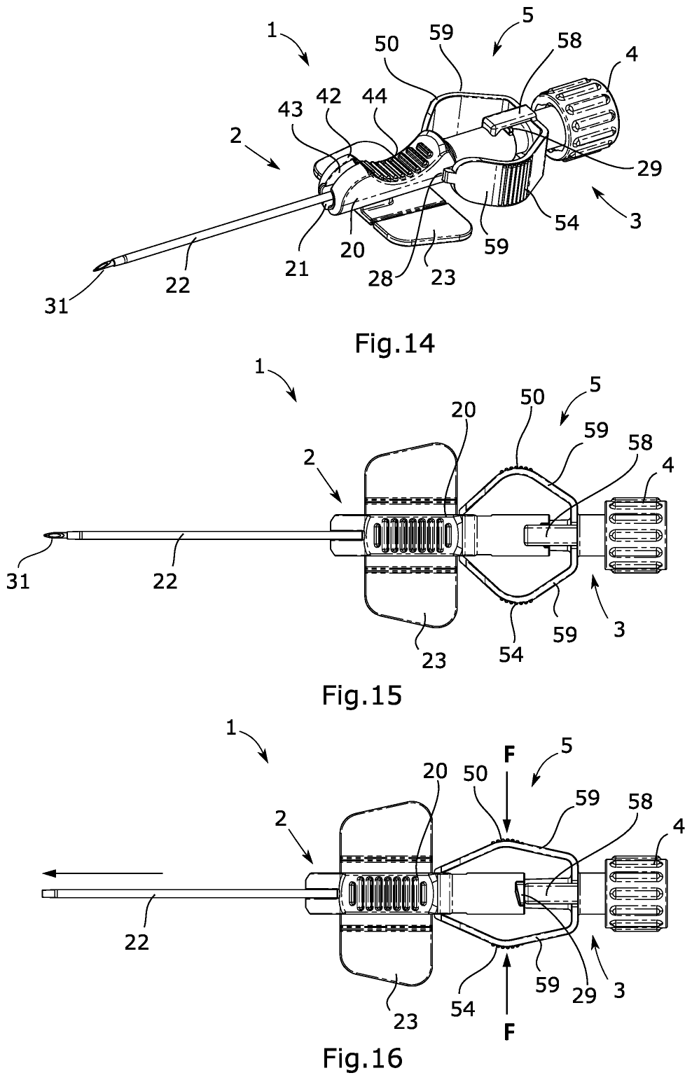

FIG. 14 shows an indwelling cannula in a further embodiment in a perspective view,

FIGS. 15, 16 show the indwelling cannula according to FIG. 14 in a plan view in various states of use,

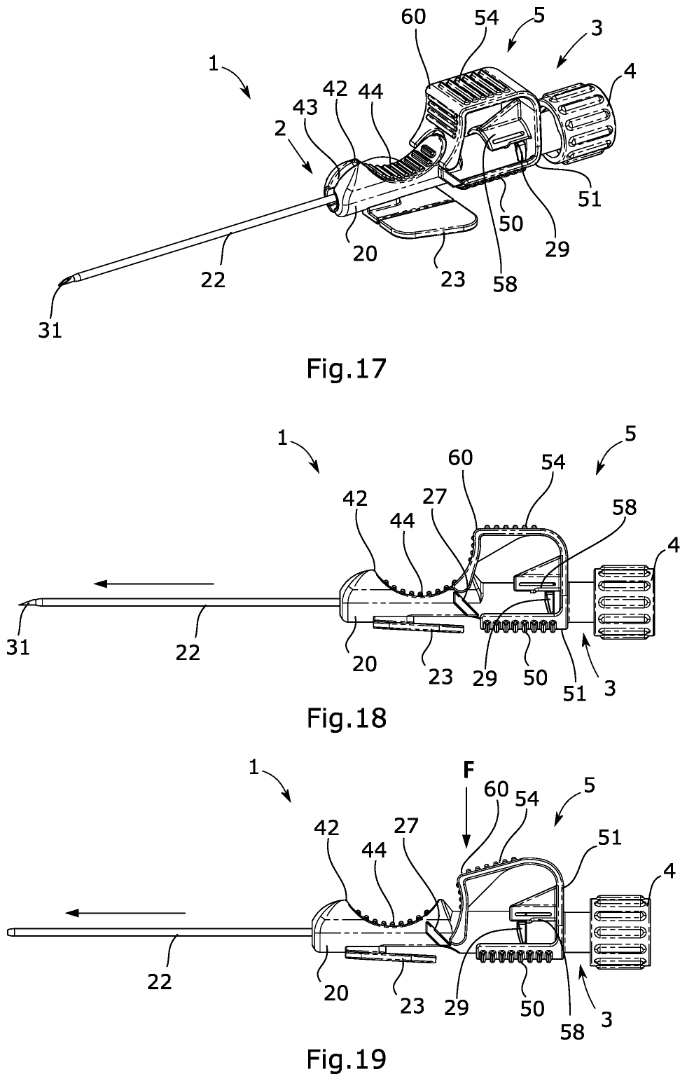

FIG. 17 shows an indwelling cannula in a further embodiment in a perspective view,

FIGS. 18, 19 show the indwelling cannula according to FIG. 17 in a side view in various states of use.

The indwelling cannula 1 represented in an exploded view in FIG. 1 has a catheter 2 and a needle device 3. The catheter 2 has a housing 20, on which there is a tube exit region 21 on a side turned toward the patient, at which a catheter tube 22 emerges from the housing 20 and projects therefrom. The catheter tube 22 is configured to be comparatively flexible and is used as an application option for the intravenous administration of liquids, in particular infusion solutions, blood products and medications. The catheter tube 22 is then located with its distal part in a hollow body, for example in the vein of a patient. The needle device 3 has a puncture needle 30, which in the basic state of the indwelling cannula 1 is located substantially in the housing 20 and the catheter tube 22, the tip 31 of the puncture needle 30 protruding from the distal end of the catheter tube 22. The needle device 3 is displaceable in relation to the catheter 2 in the longitudinal direction L.

Arranged on the housing 20, there are fixing wings 23 which are used for manual handling and securing of the catheter 2 on the patient. On the side turned away from the tube exit region 21, the housing 20 has a needle opening 25 through which the puncture needle 30 can be placed in the housing 20 and in the catheter tube 22. After installation of the indwelling cannula on the patient, the needle device 3 is removed. The needle opening 25 is then used as a connecting option, for example, for an infusion line or an aspiration element, for example a syringe.

There may also be an injection port on the housing 20, which for example projects from the housing 20 on the side turned away from the fixing wings 23. The injection port is used to inject medications. It is otherwise closed by means of a closure cap.

The needle device 3 furthermore has a closure element 33 with plugs and a handling element 32 on the proximal end of the puncture needle 30. In the basic state, the needle opening 25 is closed by the closure element 33. The handling element 32 is used for handling of the needle device 3 by the user, i.e. essentially for withdrawing the puncture needle 30 after puncture has been carried out. The needle device 3 is, as mentioned, removed after installation of the catheter 2 on the patient.

At the proximal end, i.e. in the region of the handling element 32, the needle device 3 may have an attachment port 34 for attaching a syringe or an infusion line, for example for blood aspiration. The attachment port 34 is closed by a closure cap 4. The closure cap 4 may, for example, be screwed or plugged on.

FIGS. 2 to 5 show an indwelling cannula 1 having a manually actuable forward feed mechanism 5 for generating a relative movement between the catheter tube 22 and the puncture needle 30. In this embodiment, the forward feed mechanism 5 has a base body 51 which is fastened on a housing or another part of the needle device 3, for example on the closure element 33 or in the vicinity thereof. Connected to the base body 51 by means of an articulation 52, there is a pivotably mounted lever 53 which forms an operating element of the manually actuable forward feed mechanism 5. The lever 53 is pivotably connected to the base body 51 by means of the articulation 52. The articulation 52 may for example be configured as a material bridge, for example in the form of a material bond, between the lever 53 and the base body 51. In this way, the module consisting of the base body 51, the articulation 52 and the lever 53 may be produced as an integral module. The articulation 52 may in this case form a rotation axis about which the lever 53 is pivotably or twistably mounted.

A grip face 50 for placing a finger of the user is present on the base body 51. A grip face 54, for example in the form of a concave trough with ribs, is present on the lever 53. The grip faces 50, 54 are arranged on sides of the respective constituent parts that are turned away from one another, so that for example they may be gripped between the thumb and the index finger of the user. In this case, for example, the middle finger of the user may be placed supportingly on the lower side of the base body 51, as will also be shown below with the aid of FIGS. 6 and 7. The grip faces 50, 54 may, for example, have ribs in the transverse direction. In order to provide an increased reception space for the finger of the user in the region of the front grip face 54, the housing 20 may have in front of the grip face 54 a taper or an otherwise configured trough 70 into which the finger of the user may at least partially extend during the actuation of the lever 53.

Arranged on the housing 20 of the catheter 2 or a part connected thereto, there is a housing edge or another element, for example as represented in the drawings a laterally projecting pin 57, by means of which an actuation force applied on the grip face 54 can be transmitted via the lever 53 onto the catheter 2 in order to generate the relative movement between the catheter tube 22 and the puncture needle 30. The pin 57 or the other edge may, for example, be formed integrally on the housing 20. A corresponding pin receptacle for receiving the pin 57 may be arranged, for example integrally formed, on the lever 53, which in the unactuated state of the forward feed mechanism 5 encloses the pin 57 to such an extent that manual pushing of the catheter tube 22 forward beyond the puncture needle 30 is thereby blocked. Only after actuation of the lever 53, as represented in FIG. 4, is the pin 57 released to such an extent that the desired pushing of the catheter tube 22 forward relative to the puncture needle 30 is possible.

FIG. 3 represents the indwelling cannula 1 with the forward feed mechanism 5 still unactuated. Accordingly, the puncture tip 31 protrudes from the catheter tube 22. FIG. 4 represents the indwelling cannula 1 with the forward feed mechanism 5 actuated. Accordingly, the puncture tip 31 is received in the catheter tube 22.

In order to lock the forward feed mechanism 5 in the manually actuated position, one or more locks may be present. By way of example, it is represented that a latching edge 56 is formed on the base body 51, for example an edge in a groove, which cooperates with a latching hook 55 arranged on the lever 53. In the manually actuated position, as represented in FIG. 4, the latching hook 55 latches behind the latching edge 56 so that this manually actuated position, in which the puncture tip 31 is received in the catheter tube 22, cannot further be inadvertently modified.

As represented in particular by the partially cutaway representations of FIGS. 3 and 4, an additional lock may be present between the base body 51 and the catheter module 2. For instance, a first latching element 58, for example in the form of a latching tongue exposed in a U-shape, may be formed on the base body 51, which can latch with a latching edge 29, that may be formed on the patient-remote side of the housing 20, in the manually actuated position. Inadvertent movement of the catheter tube 22 backward in relation to the puncture needle 30 is thereby additionally avoided.

At least one of the locks may be configured in such a way that an acoustic signal, for example a click sound, is generated during manual actuation of the forward feed mechanism 5 and the corresponding latching. In this way, the user may be provided with acoustic feedback relating to the complete actuation of the forward feed mechanism 5.

The housing 20 also has two protecting projections 42 extending on the left and right away from the catheter tube 22 in the distal direction. The protecting projections 42 protrude from the housing 20 on the tube exit region 21. Guide slots 43 are respectively arranged above and below the catheter tube 22 between the protecting projections 42. In particular, the lower guide slot 43 makes it possible that the catheter tube 22 in the state installed on the patient can extend in a smooth bend toward the patient, i.e. the guide slot 43 provides a space for defined bending of the catheter tube 22 and prevents kinking or breaking of the catheter tube 22. The catheter tube 22 is additionally protected laterally in this region by the protecting projections 42. A protective cap receptacle may be formed on the protecting projections 42, for example in the form of a recess extending into the protecting projections 42. In this way, a protective cap may be fitted over the puncture needle tip 31 and the catheter tube 22 and be fastened on the housing 20.

Additional grip faces on the housing 20 may extend toward the distal end into the region of the protecting projections 42. Advantageously, a finger stop device is arranged at the distal end of the housing 20, or of the protecting projections 42, for example in the form of a thickening, for example a bead or a flange. This prevents the user from slipping forward with their fingers toward the patient 5 from the housing 20, or from the grip faces, for example when installing the indwelling cannula on the patient.

Further grip faces may also respectively be formed laterally on the base body 51. These grip faces may respectively be fitted laterally so that it is no longer possible to “peep” from the side through the base body 51. Intuitively, these grip faces may for example be gripped with the right thumb (on the left) and the right middle finger (on the right), the right index finger then being capable of operating the lever 53. A corresponding exemplary embodiment will also be explained below with the aid of FIGS. 9 to 13.

In other regards, the indwelling cannula 1 according to FIGS. 2 to 5 may be configured in the same way as the indwelling cannula 1 according to FIG. 1.

FIGS. 6 to 8 show the practical use of the indwelling cannula 1 by a user, only one hand 6 of whom is represented. In FIG. 6, the indwelling cannula 1 is in the initial state in which the puncture tip 31 protrudes from the catheter 22. In this state, as in the case of conventional indwelling cannulas, the puncture of the hollow body to be punctured is carried out. Once the puncture has been carried out, i.e. the puncture tip 31 together with the near-patient end of the catheter tube 22 is inside the hollow body, the manual actuation represented in FIG. 7 of the forward feed mechanism 5 may be carried out. For this purpose, a force is transmitted onto the lever 53 by means of the grip face 54, in which case the hand 6 may be supported on the opposite grip face 50, for example by means of the thumb. It may be seen in FIG. 7 that the puncture tip 31 is now received in the catheter tube 22. The puncture needle 30 in this case remains stably in position when the forward feed mechanism 5 is actuated.

The indwelling cannula 1 may in this case advantageously be gripped and operated with one hand, for example by the thumb of the hand 6 being placed on the grip face 50, the index finger being placed on the grip face 54 and in addition a further finger, for example the middle finger, optionally being placed on the lower side of the base body 51 for support.

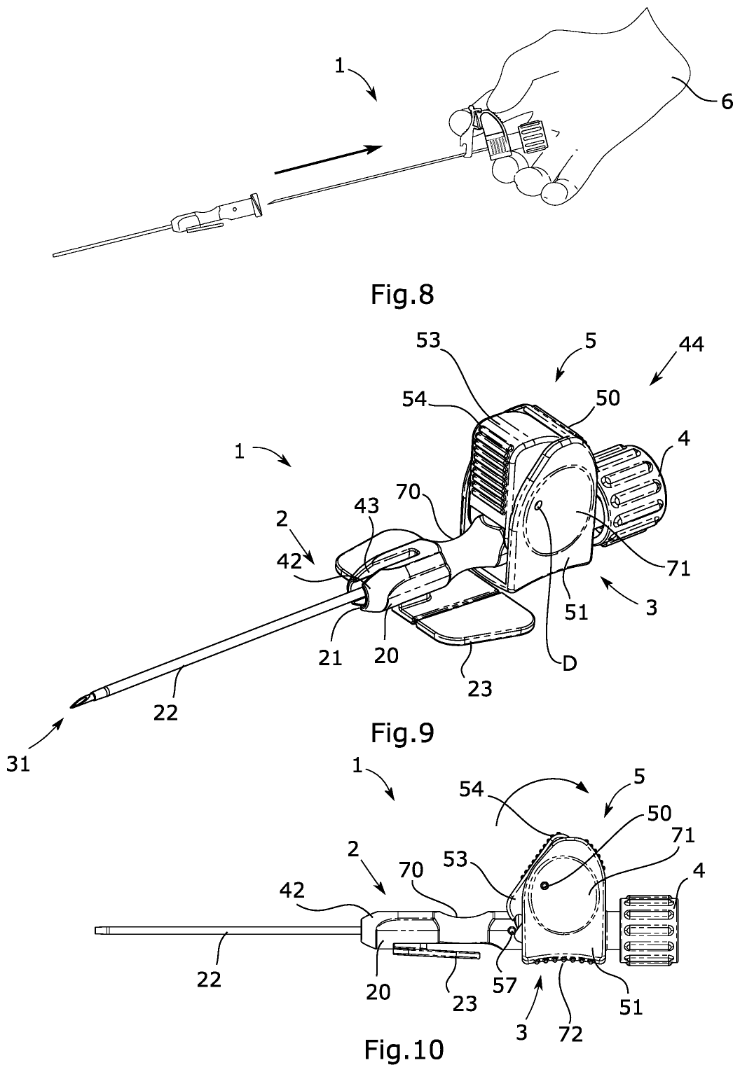

After the relative movement generated by the forward feed mechanism 5 has been carried out, the catheter tube 22, which is braced at least substantially over its entire length by the inner-lying puncture needle 30, is pushed further into the hollow body until a desired position is reached. Subsequently, as in the case of conventional indwelling cannulas, the needle device 3 is removed from the catheter 2, in which case it may be seen in FIG. 8 that the forward feed mechanism 5 is thereby removed at the same time.

In the embodiment described so far, the forward feed mechanism 5 is configured to be relatively open laterally in the region of the grip face 50. This part may however also be configured to be laterally closed, for example by side walls. Additional lateral grip faces may be present in the region of the side walls.

With the aid of the embodiment of the indwelling cannula according to FIGS. 9 to 13, a variant of the forward feed mechanism 5 which has a pivotable lever 53, in a similar way to the embodiment described above, will be described. This lever, however, is arranged in a base body 51 which is at least substantially laterally closed and is mounted on a rotation axis D. The base body 51 encloses the lever 53 at least laterally and partly also rearward, so that the lever 53 is substantially covered from the surroundings and primarily the grip face 54 lies outward for the manual actuation. In particular, the locking mechanism with the latching hook 55 and the latching edge 56 may be arranged inside the region enclosed by the base part 51. In this way, the parts of the forward feed mechanism 5 and the needle opening 25 are protected even better against contamination. It also, for example, avoids part of the glove of the user being capable of being trapped in the region of the locking elements 55, 56.

As may be seen in FIG. 9, the grip face 54 extends as seen from the puncture needle 30 from a position below the rotation axis D, i.e. a position arranged closer to the puncture needle 30 than the rotation axis D, to a position arranged above the rotation axis D.

The operation of the indwelling cannula 1 and its forward feed mechanism 5 during application on the patient may be improved even further in the variant shown by lateral grip faces 71 being arranged on both sides on the base part 51. In addition, there may also be a lower grip face 72 on the lower side of the base body 51, i.e. the side turned toward the patient. The lateral grip faces 71 and/or the lower grip face 72 may likewise have corresponding structuring, as already explained for the grip faces 50, 54.

The base body 51, on which the lever 53 is fastened, may be configured as an integral structural unit with a housing of the needle device 3 or the closure element 33, for example as a plastic injection-molded constituent part.

The base body 51 may, for example, be configured as a substantially closed housing which receives the lever 53. For example, the base body 51 may have at least two opposite side walls, a lower wall as well as a rear wall turned toward the proximal end. A side of the base body 51 which is open toward the distal end may then be substantially closed by the lever 53.

On a side turned toward the base body 51, the lever 53 may have a rear wall which has a circularly bent contour at least on the outer side. A rear wall of the base body 51 turned toward the circularly bent contour of the lever 53, for example a rear wall which has the grip face 50, may have a contour complementary thereto, i.e. a likewise circularly curved contour, at least on the inner face turned toward the curved contour of the lever 53. The circularly curved contours may in this case be arranged concentrically with respect to the rotation axis of the lever 53. In this way, the lever 53 is configured so speak nested with the base body 51 configured as a housing, which leads to particularly good protection against contamination. In addition, the lever 53 may have side walls which are arranged parallel to side walls of the base body 51 and which overlap at least partially with the side walls of the base body 51. In this way, the arrangement consisting of the base body 51 and the lever 53 also forms good contamination protection in the lateral direction.

The lever 53 may in this case be mounted twistably about its rotation axis D on the base body 51. In order to form the rotational mounting, for example, a respective pin that engages into an indentation on the respective other constituent part may be formed on one of the parts constituted by the lever 53 and the base body 51.

FIG. 9 shows the forward feed mechanism 5 in the unactuated position. FIG. 10 shows the forward feed mechanism 5 in the actuated position.

In a similar way thereto, FIG. 11 shows the forward feed mechanism 5 in the unactuated position and FIG. 12 shows it in the actuated position. It may be seen that the locking elements 55, 56 are arranged in the region surrounded by the base body 51, so that they are protected against environmental influences and are tamper-proof. In FIG. 12, the latching hook 55 is latched with the latching edge 56.

Because of the various grip faces, there are a multiplicity of options for the user to hold and operate the indwelling cannula 1. FIG. 13 shows an advantageous variant of the operation, in which the indwelling cannula 1 is operated with three fingers, for example in such a way that the user grips the indwelling cannula 1 on the opposite lateral grip faces 71 with the thumb and middle finger and places the index finger on the grip face 54 of the lever 53. In this way, the puncture of the hollow body with the puncture tip 31 may initially be carried out and then the forward feed mechanism 5 may be actuated with the index finger via the grip face 54 without the position of the hand 6 having to be changed.

FIGS. 14 to 16 show an indwelling cannula 1 with an alternative configuration of the manually actuable forward feed mechanism 5. In this embodiment, the forward feed mechanism 5 is configured in a pincer-like form with two mutually opposite pincer arms 59, between which a part of the housing 20 of the catheter 2 extends. Here as well, there are again grip faces 50, 54 turned away from one another, which in this case are formed on the outer sides of the pincer arms 59. The pincer arms 59 are firmly connected on one side to the needle device 3, and on their other side, i.e. at the opposite free end, the pincer arms engage into corresponding indentations in the housing 20; they are supported on housing edges 28 and via these housing edges 28 during manual actuation, as represented in FIG. 14, they ensure that the catheter 2 and therefore the catheter tube 22 are pushed forward in relation to the needle device 3.

FIG. 15 represents the indwelling cannula 1 with the forward feed mechanism 5 still unactuated. Accordingly, the puncture tip 31 protrudes from the catheter tube 22. FIG. 16 represents the indwelling cannula 1 with the forward feed mechanism 5 actuated. Accordingly, the puncture tip 31 is received in the catheter tube 22. The actuation is carried out by applying actuation forces F turned toward one another on the grip faces 50, 54, for example by means of the thumb and index finger of one hand.

In order to lock the forward feed mechanism 5 in the manually actuated position in which the puncture tip 31 is received in the catheter tube 22, a latching arm 58, which cooperates by latching with a latching edge 29 that is formed on the housing 20, may for example be provided on the needle device 3, for example in the region on which the pincer arms 59 are connected to the housing of the needle device 3.

FIGS. 17 to 19 show an indwelling cannula 1 with an alternative configuration of the forward feed mechanism 5. The forward feed mechanism 5 in this case has a base body 51 which, in a similar way to the embodiments described above, is fastened on a constituent part of the needle device 3. The base body 51 extends in the direction of a pressing arm 60, by means of which a pressing force can be exerted onto the housing 20 of the catheter 2. For the manual operation, there is a grip face 50 on the lower side of the base body 51 and, turned away therefrom, a grip face 54 on the pressing arm 60. The user then grips the indwelling cannula 1 with two fingers, for example the thumb and index finger, between the grip faces 50, 54.

If the desired relative movement between the catheter tube 22 and the puncture needle 30 is intended to be generated by the forward feed mechanism 5, a force F is pressed from above via the grip face 54 onto the pressing arm 60. The latter then transmits a force onto a forward feed face 27 configured as an oblique plane, which is formed on the housing 20. The forward feed face 27 need not necessarily be configured as a plane, but may also be curved. By the force F exerted from above by the pressing arm 60 onto the forward feed face 27, as shown in FIG. 19, the catheter tube 22 is pushed forward relative to the puncture needle 30 so that the puncture tip 31 is received in the catheter tube 22.

FIG. 18 represents the indwelling cannula 1 with the forward feed mechanism 5 still unactuated. Accordingly, the puncture tip 31 protrudes from the catheter tube 22. FIG. 19 represents the indwelling cannula 1 with the forward feed mechanism 5 actuated. Accordingly, the puncture tip 31 is received in the catheter tube 22.

In order to lock the forward feed mechanism 5 in the manually actuated position, there is again a latching arm 58 on the base body 51, which in the manually actuated setting latches behind a latching edge 29 that is arranged on the housing 20.

### 2ND EMBODIMENT

### Indwelling Cannula

The invention relates to an indwelling cannula for puncturing a hollow body by means of a puncture needle, wherein the indwelling cannula has at least one catheter with a vasiform catheter tube, in which the puncture needle can be guided longitudinally displaceably, wherein the catheter is adapted to be pushed over at least a part of the length of the catheter tube through the opening created by means of the puncture needle through the tunic of the hollow body to be punctured after puncture of a tunic of the hollow body to be punctured has been carried out, and to remain there over a period of time.

Such an indwelling cannula may, for example, be configured as an indwelling venous cannula. The hollow body to be punctured may be a hollow body of a living being or a hollow body of an object. If the indwelling cannula relates to the medical field, the hollow body may for example be a blood vessel, for example a vein or artery.

The terms “puncturing” and “puncture” are in this case to be understood in the medical sense. “Puncturing” refers to the entry of a puncture needle into the hollow body, in such a way that the puncture needle penetrates through the tunic of the hollow body to be punctured.

Those parts which are arranged at the remote (distal) end of the indwelling cannula, from the viewpoint of the user, and therefore in the vicinity of the punctured hollow body, are regarded as a section of parts of the indwelling cannula near to the hollow body. Accordingly, sections remote from the hollow body are arranged at the proximal end of the indwelling cannula from the user's viewpoint, i.e. further away from the punctured hollow body. In connection with medical applications of the indwelling cannula, the terms “near-vein” or “near-patient” or “user-remote” are also used synonymously with the term “near to the hollow body”, and “vein-remote” and “patient-remote” and “near-user” are also used synonymously for the term “remote from the hollow body”.

Near-vein components tend to be located inside the patient, and vein-remote components tend to be arranged outside the patient. This need not necessarily always apply, but is meant to further clarify the terms. The additional elements are generally self-evident, and the context is essential.

A piercing-resistant indwelling venous cannula is disclosed by PCT/EP2019/057097.

An improved puncture system in the sense of a general puncture system is intended to be provided. In principle, with the improved indwelling cannula it is likewise possible advantageously to puncture all body cavities and interstitial body spaces and all anatomical and morbid structures which are intended to be punctured, and to install a catheter in them.

Components of the improved indwelling cannula may in principle also be combined with all known puncture and catheter systems or be used as independent products.

Although the term “indwelling cannula” will be adopted below, it has a wider meaning in the sense of a general puncture system with which not only veins may be punctured. In what follows, the terms “vein” and “veins” thus include in principle all blood vessels and very generally all body cavities and interstitial body spaces and all anatomical and morbid structures which are intended to be punctured and provided with a catheter.

Thus, in addition, by way of example the trachea, pleural space, abdomen, stomach, intestine, renal pelvis, urinary bladder, cerebrospinal fluid space and bone may be punctured with the indwelling cannula. In addition, morbid structures such as abscesses in and on the patient may be punctured. Advantageously, arterial blood vessels may also be punctured.

In what follows, the term “patient” includes all living beings of all age groups and genders. Applications in technical fields and in and on all objects and structures, for example in and on reservoirs, containers, cavities, extensible materials and in or on pump, hose, pipe and port systems, are likewise explicitly possible.

When the word “or” is used in what follows, possible alternatives are indicated but combinations of the features or configurations separated by “or” are also fundamentally explicitly possible.

All components described may be employed singly or multiply on an indwelling cannula or also, independently of the latter, on/in other products or entirely independently. Various features of different components are also freely combinable, and features of particular components may also be used in other components without this being explicitly mentioned. All components and features may fundamentally be employed inside and outside a patient.

Indwelling cannulas, and in particular indwelling venous cannulas, are special medical devices insofar as they must have particular diameter and length proportions in order to be able to be applied in human and animal patients at the usual venous access sites. For this purpose, a certain flexibility or resilience of the catheter tube is also necessary. Such indwelling cannulas are therefore dissimilar to catheter systems for other applications, for example a urinary catheter, because entirely different requirements apply in those cases.

Table 1 below gives an overview of the currently usual variants of indwelling venous cannulas, primarily for the field of human medicine.

| | Outer | Inner | Wall | | | |
|---|---|---|---|---|---|---|
| | diameter | diameter | width | Length | Color | Flow rate |
| Gauge | [mm] | [mm] | [mm] | [mm] | code | [ml/min] |
| 14G | 2.1 | 1.7 | 0.2 | 45 | orange | 340 |
| 16G | 1.8 | 1.25 | 0.2 | 45 | gray | 180 |
| 18G | 1.3 | 0.9 | 0.2 | 32 | green | 90 |
| 20G | 1.1 | 0.7 | 0.2 | 32 | pink/ | 60 |
| | | | | | roseate | |
| 22G | 0.9 | 0.55 | 0.08 | 25 | blue | 36 |
| 24G | 0.7 | 0.45 | 0.08 | 19 | yellow | 20 |
| 26G | 0.6 | 0.35 | 0.08 | 19 | violet | 13 |
| Bandwidth | ±20% | ±20% | ±20% | ±60% | | ±50% |

The dimensions of outer diameter, inner diameter, wall width and length refer to the catheter tube. The column “Bandwidth” indicates the tolerance range which is possible for the respective specifications.

As may be seen, the catheter tube has relatively small diameter dimensions and correspondingly also a very small wall width. Usual catheter tubes are produced from homogeneously extruded plastic materials. With the dimensions mentioned, the plastic material used gives rise to the desired flexibility. However, such catheter tubes have only a limited resistance against transverse forces, i.e. the stability in relation to compression of the catheter tube, with the effect of an undesired reduction of the effective inner cross-sectional area and therefore the possible flow rate of liquids, is relatively low. In particular application situations, kinking of the catheter tube may also occur, so that the flow may be fully blocked or at least almost fully prevented at the kink site.

The object of the invention is to provide an indwelling cannula which is less sensitive to transverse forces and/or kinking.

This object is achieved by an indwelling cannula as claimed in claim 1. Advantageous developments are specified in the dependent claims.

According to one advantageous configuration of the invention, the catheter tube has at least one supporting structure by which the bending moment of resistance and/or the radial moment of resistance of the catheter tube is increased. The supporting structure may be configured in the form of one or more reinforcing elements.

The supporting structure may, for example, be a spiral structure extending over a certain length section of the catheter tube, which may be integrated on the inner side and/or on the outer side of the catheter tube and/or partially or fully into the catheter tube wall. There may also be a plurality of coaxial supporting structures of this type. The spiral structure may also have spring-like properties. Instead of a spiral structure, there may also be individual supporting rings spaced apart from one another, integrated both on the inner side, the outer side and/or wholly or partially into the catheter tube wall. The spiral structure and/or the rings may be formed from round material or flat material, in particular from a metal material. At least one supporting structure may also be formed by a thin-walled supporting tunic or a supporting braid consisting of fibers interlaced with one another or fibers joined to form a woven or laid fabric. The catheter tube may be configured in a single ply or multiple plies in the radial direction, both from the same and from different materials in the individual radial plies. For example, a supporting structure may be embedded into an inner radial ply which is covered by an outer radial ply of the catheter tube.

For example, polyurethane (PU) or FEP may be envisioned as materials for the catheter tube. Materials for the supporting structure may be metal materials, in particular stainless steel and/or metal alloys, in particular nitinol. Nitinol has the advantage that the desired resistance of the catheter tube against compression or kinking is increased further by the memory effect.

Further materials for the supporting structure may be plastic materials, in particular also fiber-reinforced, for example carbon- or aramid fiber-reinforced plastics.

In the case of a spiral configuration of the supporting structure, there may be one spiral or a plurality of spirals placed in one another. The turns of the spirals may have the same spacing in front of one another over the entire length of the supporting structure, or varying spacings. In one advantageous configuration of the invention, a spiral supporting structure may have a smaller spacing of the turns in front of one another in the region remote from the hollow body than in the region near to the hollow body.

There may also be further structures, which stabilize the supporting structure, between the turns. In one advantageous version, these structures are tensioned when the turns move away from one another, and thus become more rigid. The structures between the turns also ensure that the turns can only be spaced apart limitedly from one another. When the turns move toward one another, the structures between them become less rigid. In this version, the supporting structure has a structure similar to an accordion.

In one advantageous version, the structures between the turns may form the supporting structure at least partially fluid-impermeably with a corresponding material selection.

By such a supporting structure, when using a material that is visible in imaging medical methods, in particular a metal material, the visibility of the catheter in patients by means of imaging medical methods, for example by ultrasound scanning, may be improved. In one advantageous configuration of the invention, the supporting structure or the sequence of a plurality of supporting structures in the longitudinal direction is configured nonuniformly over the longitudinal extent. The nonuniform configuration makes even more accurate position identification of particular regions of the catheter in the patient possible by means of imaging medical methods.

According to one advantageous configuration of the invention, the catheter tube has a multiplicity of lateral passage openings which extend from the inner side to the outer side. The passage openings (side holes) may be present over the entire length of the catheter tube or only over one or more longitudinal sections, for example in a section near to the hollow body. The passage openings may be arranged distributed over the circumference of the catheter tube. The flow of liquids through the catheter tube can be improved by the passage openings in the catheter tube wall.

According to one advantageous configuration of the invention, one, several or all of the lateral passage openings are arranged in the region of the catheter tube not covered by the supporting structure. If the supporting structure has a spiral shape, for example, passage holes may be arranged between the turns of the spiral. In this way, the flow through the passage openings is not impeded by the supporting structure.

It is also conceivable that a spiral supporting structure is not enclosed by and/or coated with a fluid-impermeable material at least over a partial length of the catheter tube. The indwelling cannula may thereby be permeable for liquids in this region even without passage holes. In one advantageous configuration, the turns of the spiral supporting structure have a larger spacing from one another in this region than in the other regions of the catheter tube. This region may, as described above, preferentially lie near to the hollow body.

It is also conceivable that a fluid-impermeable material enclosing and/or coating the spiral supporting structure has gaps and/or interruptions at least over a partial length and therefore ensures fluid permeability of the indwelling cannula in the region of these gaps/interruptions. Side holes are then no longer absolutely necessary.

In a further advantageous version, the aforementioned material may also only partially enclose the spiral supporting structure continuously in the longitudinal direction of the indwelling cannula. For instance, a kind of web which counteracts uncoiling of the supporting structure may be formed. In the case of very tight winding, the supporting structure may be configured to be fluid-impermeable, while in the case of less tight winding it may however also be configured to be fluid-permeable. The tightness of the winding may also determine whether the supporting structure is, for example, configured to be fluid-impermeable but nevertheless permeable for gases/vapors. It is also conceivable for the supporting structure to be permeable only for particular fluids. It is likewise conceivable for the supporting structure to have thermoplastic properties and/or to expand due to body heat.

According to one advantageous configuration of the invention, the catheter has a housing on which the catheter tube is fitted. The catheter tube extends from a side of the housing directed toward the hollow body. According to one advantageous configuration of the invention, in the region in which the catheter tube projects from the housing, i.e. in a tube exit region of the housing, the catheter has a kink protection structure circumferentially enclosing the catheter tube fully or at least partially, by which the risk of the catheter tube kinking in the region of the exit site from the housing is reduced. In this way, undesired kink formation of the catheter tube when manipulating the indwelling cannula can be avoided, particularly in the region of the exit site from the housing. This region is particularly susceptible to kinks since, in known indwelling venous cannulas, an abrupt transition takes place from the relatively robust inflexible housing into the comparatively thin-walled and correspondingly sensitive catheter tube. By the kink protection structure according to the invention, the hitherto abrupt transition in this region is made gentler. The kink protection structure may, for example, involve a surface rounded in the shape of a trumpet, which circumferentially encloses the catheter tube.

According to one advantageous configuration of the invention, it is provided that the housing has protecting projections for protecting the catheter tube, which extend from the tube exit region of the housing in the distal direction, at least one slot running in the longitudinal direction being present between the protecting projections being present at least on the patient application side of the indwelling cannula, which is at least as wide as the diameter of the catheter tube so that the catheter tube can extend in a uniform arc through the slot when the indwelling cannula is applied on the patient. In this way, kink protection and at the same time breaking protection is produced for the catheter tube in the region of the exit site from the housing. By the protecting projections extending at least some way in the longitudinal direction, the catheter tube is also protected laterally. A slot, through which the catheter tube may likewise extend, may additionally be present on the side of the housing turned away from the patient application side. The patient application side of the indwelling cannula is to be understood as the side with which the indwelling cannula is to be fastened on the patient because of its design, for example the lower side of fixing wings.

The length of the protecting projections and/or of the slot may be at least ten times the diameter of the catheter tube. In this way, a relatively large guide length is provided for the catheter tube in the longitudinal direction between the protecting projections. In addition, protection of the catheter tube against environmental influences may also be provided in the sensitive transition region from the housing of the indwelling cannula into the patient.

According to one advantageous configuration of the invention, the indwelling cannula has a grip region which is adapted in such a way that the indwelling cannula is gripped in the grip region during manipulation by the user. The grip region of the indwelling cannula may in this case be a region in which there are no other functions or functional elements of the indwelling cannula, but which is provided only for manual gripping of the indwelling cannula. This has the advantage that the user intuitively grasps the indwelling cannula at the proper site, so that unnecessary touching at other sites is avoided, for example at sites of the indwelling cannula which must necessarily remain sterile. In this way, unnecessary contaminations of particular sites of the indwelling cannula can be avoided. For example, the grip region may be formed on the housing of the catheter, for example shaped integrally with the housing. The grip region may, for example, be produced by a corresponding configuration of regions of the outer surface of the housing.

The indwelling cannula may also be equipped with a separate constituent part, i.e. a grip piece, which may be removed after installing the indwelling cannula on the patient. If such a grip piece is present, the grip region is located on this grip piece. Such a grip piece may be configured in such a way that it at least partially or predominantly encloses the structures of the indwelling cannula which come to lie outside the patient after the indwelling cannula is installed. The grip piece may also fully enclose these structures, however. In one advantageous configuration, the grip piece also encloses the catheter tube over a partial length in the region remote from the hollow body, in particular where the catheter tube projects from the housing, i.e. in a tube exit region of the housing. This region is therefore protected against contamination with germs.

The grip piece may be removed by pulling it upward from the indwelling cannula (away from the patient). In one advantageous configuration, however, the grip piece may also be pulled away rearward in the direction of the user and thereby removed. The grip piece may be configured in one part or several parts. In a multi-part version, individual parts of the grip piece may selectively be removed, left or rejoined to the indwelling cannula. Special situations, in which only defined subregions of the indwelling cannula require particular protection, may therefore be taken into account.

In one advantageous configuration of the invention, the grip region is configured in such a way that it has two grip faces, which are arranged on mutually opposite sides of the constituent part on which the grip region is present. This has the advantage that the indwelling cannula can be gripped well straightforwardly and securely on the grip region with the thumb and another finger, for example the middle finger or the index finger. The grip faces may, in particular, be predominantly planar grip faces or have an at least predominantly planar part. In their planar regions, the grip faces may be arranged parallel to one another. The grip faces may also be configured in the manner of a trough with an at least locally curved (concave) surface. The grip faces may have structuring by which the indwelling cannula can be gripped even better and slipping of the fingers is avoided.

For example, ribs, pimples or other surface structuring may be present on the grip faces. The grip faces may, in particular, be configured in such a way that their dimension in the longitudinal direction of the indwelling cannula is greater, for example at least two times as great, as the dimension in the transverse direction. In this case, a respective grip face may be formed from the material of the housing of the indwelling cannula or from another material, in particular from a material having greater resilience or flexibility. For example, the grip face may be configured as an insert, for example made of an elastomer, for example rubber, or silicone, or any other material softer than the material of the housing of the indwelling cannula. Such an insert may then be fastened in a corresponding indentation of the housing of the indwelling cannula.

According to one advantageous configuration of the invention, the indwelling cannula has at least one finger stop device on the housing, which is arranged on the tube exit region or near to the tube exit region. The finger stop device is therefore spaced apart significantly from the proximal end of the housing. The finger stop device is used to avoid accidental slipping of the fingers of the user, with which the user grasps the indwelling cannula during application on the patient, from the housing in the longitudinal direction of the indwelling cannula, i.e. slipping toward the patient. The finger stop device therefore forms an obstacle for the fingers against slipping from the housing in the direction of the patient. In this way, contaminations of the entry site on the patient and of parts of the indwelling cannula in the region of the entry site are avoided. The finger stop device may, for example, be configured as a circumferential enlargement in relation to neighboring circumferential regions of the housing, for example in the form of a flange, a bead or another thickening. The finger stop device may circumferentially enclose the housing fully or only in subsections, for example only laterally on the right and left of the catheter tube.

According to one advantageous configuration of the invention, the indwelling cannula has at least one adhesive fastening element for self-adhesive fastening of the indwelling cannula on the patient. The adhesive fastening element may be covered by a protective film on the adhesive side in the delivery state of the indwelling cannula. If the indwelling cannula is intended to be fixed on the patient, the protective film may be peeled off straightforwardly and the indwelling cannula may then be fastened directly on the patient with the adhesive fastening element. The protective film may be developed with structures that project beyond it, in particular projecting laterally beyond it. These structures may, for example, be configured as tabs which do not themselves have any adhesive properties. The protective film may then be peeled away from the indwelling cannula at the tabs without its having to be lifted for the peeling process. This may improve the patient comfort and also be useful to avoid traumatization of anatomical structures by unnecessary movements of the indwelling cannula. The use of such a protective film has the advantage that the user does not need to provide separate adhesive fastening means in order to fasten the indwelling cannula on the patient, example plasters or the like.

The adhesive fastening element may, for example, be arranged as an adhesive coating on the patient application side of fixing wings of the indwelling cannula or as a separate adhesive strip which is applied on the side of the fixing wings turned away from the patient application side.

As an alternative or in addition to an adhesive fastening element, there may also be at least one needle-like or spiral structure with which the indwelling cannula can be fastened on the skin. This may be enclosed by a protective cap which can be actively removed by the user in order to avoid injury to the patient and the user.

According to one advantageous configuration of the invention, fixing wings for fixing the indwelling cannula on the patient are arranged on the housing. The fixing wings are connected to the housing by means of a material bridge. The material bridge does not in this case extend over the full length extent of the fixing wing in the longitudinal direction, but has a recess or interruption through which an adhesive strip, with which the indwelling cannula can be fastened on the patient, can be passed. For example, the recess may be arranged on a section of the fixing wings directed toward the proximal end of the indwelling cannula, i.e. the material bridge is then located on the side of the fixing wings directed toward the distal end.

According to one advantageous configuration of the invention, the indwelling cannula has at least one fixing wing or an arrangement of fixing wings, which is coupled by means of a movement mechanism to the housing and by which the location of the entire fixing wing or of the entire arrangement of fixing wings in relation to the housing can be readjusted. This has the advantage that the fixing wing or the arrangement of fixing wings may initially be arranged when installing the indwelling cannula on the patient at a position which is particularly suitable for the installation, and may be readjusted into a position which is particularly suitable for fastening purposes after installation on the patient. For example, the fixing wing or the arrangement of fixing wings may be fastened longitudinally displaceably on the housing, so that the fixing wing or the arrangement of fixing wings can be pushed forward in relation to the housing, i.e. toward the patient, after installation on the patient. The fixing wing or the arrangement of fixing wings may, for example, also be connected to the housing by means of a flap mechanism, by means of which the fixing wing or the arrangement of fixing wings can be pivoted into a location which is suitable for fastening on the patient. In particular, the fixing wing or the arrangement of fixing wings may be pivotable about a pivot axis which runs transversely to the longitudinal direction of the indwelling cannula.

The invention will be explained in more detail below with the aid of exemplary embodiments with the use of drawings, in which:

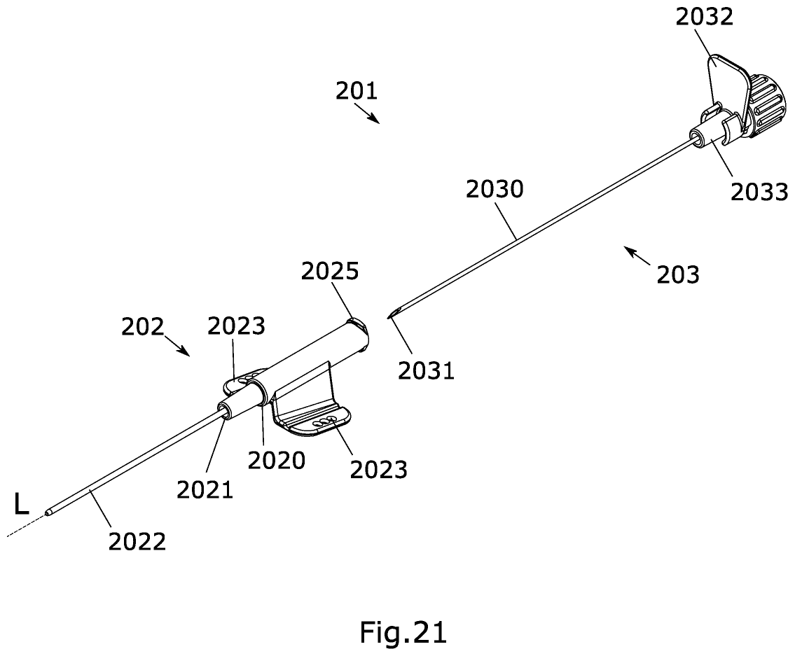

FIG. 21 shows an indwelling cannula in an exploded view,

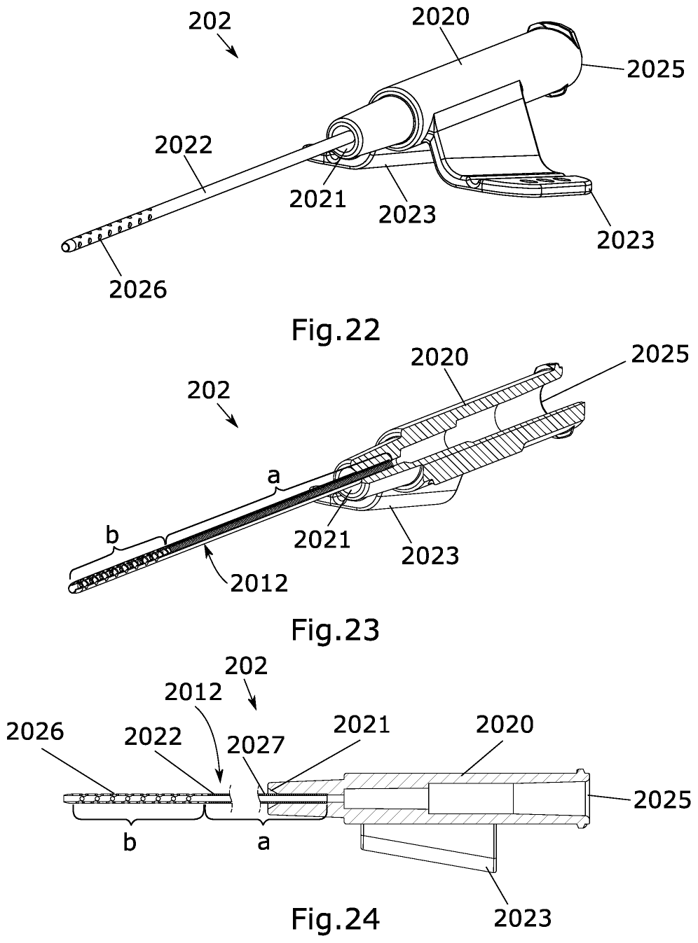

FIG. 22 shows an indwelling cannula in a perspective view,

FIG. 23 shows the indwelling cannula according to FIG. 22 in a cutaway representation,

FIG. 24 shows the indwelling cannula according to FIG. 22 in a lateral sectional view,

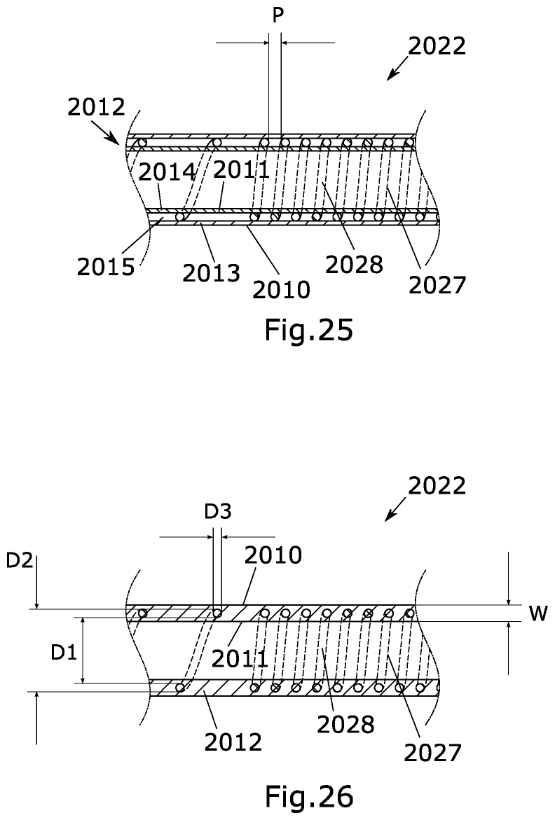

FIGS. 25 to 27 show lateral sectional views of sections of the catheter tube,

FIGS. 28 to 50 show further embodiments of indwelling cannulas.

The indwelling cannula 201 represented in an exploded view in FIG. 21 has a catheter 202 and a needle device 203. The catheter 202 has a housing 2020, on which there is a tube exit region 2021 on a side turned toward the patient, at which a catheter tube 2022 emerges from the housing 2020 and projects therefrom. The catheter tube 2022 is configured to be comparatively flexible and is used as an application option for the intravenous infusion administration of liquids, in particular medications. The catheter tube 2022 is then located with its distal part in a hollow body, for example in the vein of a patient. The needle device 203 has a puncture needle 2030, which in the basic state of the indwelling cannula 201 is located substantially in the housing 2020 and the catheter tube 2022, the tip 2031 of the puncture needle 2030 protruding from the distal end of the catheter tube 2022. The needle device 203 is displaceable in relation to the catheter 202 in the longitudinal direction L.

Arranged on the housing 2020, there are fixing wings 2023 which are used for manual handling and securing of the catheter 202 on the patient. On the side turned away from the tube exit region 2021, the housing 2020 has a needle opening 2025 through which the puncture needle 2030 can be placed in the housing 2020 and in the catheter tube 2022. After installation of the indwelling cannula on the patient, the needle device 203 is removed. The needle opening 2025 is then used as a connecting option, for example, for an infusion line or an aspiration element, for example a syringe.

There may also be an injection port on the housing 2020, which for example projects from the housing 2020 on the side turned away from the fixing wings 2023. The injection port is used to inject medications. It is otherwise closed by means of a Luer lock closure cap.

The needle device 203 furthermore has a sealing plug 2033 and a handling element 2032 on the proximal end of the puncture needle 2030. In the basic state, the needle opening 2025 is closed by the sealing plug 2033. The handling element 2032 is used for handling of the needle device 203 by the user, i.e. essentially for withdrawing the puncture needle 2030 after puncture has been carried out. The needle device 203 is, as mentioned, removed after installation of the catheter 202 on the patient.

FIGS. 22 to 24 show an advantageous embodiment of a catheter 202 in various views. In the catheter 202 represented, the catheter tube 2022 is supported by a supporting structure 2027 in the radial direction. The supporting structure 2027 may be configured for example as a spiral wire which is arranged internally or externally on the wall 2012 of the catheter tube 2022 or is integrated into the wall 2012. The spiral wire is wound more closely in a longitudinal section a than in a longitudinal section b, i.e. in the longitudinal section b there are greater spacings between the individual turns of the spiral than in the longitudinal section a. For example, the longitudinal section a may extend from a region inside the housing 2020 over, for example, from 60 to 90% of the length of the catheter tube 2022 toward the distal end. The longitudinal section b begins at the end of the longitudinal section a and ends near to the distal end of the catheter tube 2022. In the longitudinal section b, passage openings 2026 may additionally be arranged in the wall of the catheter tube 2022, i.e. lateral passage openings (side holes) through which liquid can emerge laterally even before reaching the distal end of the catheter tube 2022.

The desired liquid permeability of the catheter tube 2022 may also be achieved by a greater distance between the turns of the spiral wire if no coating is present in this region. In this case, the liquid permeability may be achieved even without the lateral passage openings.

FIG. 25 shows an advantageous embodiment of the construction of the catheter tube 2022 in the region of the transition from the longitudinal section a into the longitudinal section b. In the embodiment represented, the wall 2012 of the catheter tube 2022 is constructed in multiple layers. There is an inner layer 2014, which at the same time forms the inner side 2011 of the wall 2012. There is further an outer layer 2013, which is arranged outside the inner layer 2014 and forms the outer side 2010 of the wall 2012. Between the inner layer 2014 and the outer layer 2013, there may be an intermediate space 2015 in which the supporting structure 2027 is arranged. The regions of the intermediate space 2015 which are not filled by the supporting structure 2027 may be filled with another material. The spiral supporting structure 2027 may be a round-wire or flat-wire spiral, or a combination thereof. In the longitudinal section a, the spacing P between the individual turns of the spiral may, for example, lie in the range of from 0 to 2 millimeters. The spiral may also have varying pitches or a plurality of spirals with different pitches.

FIG. 26 shows an embodiment of the catheter tube 2022 with a supporting structure 2027 which is integrated directly into the wall 2012 of the catheter tube 2022. It may for example be produced by a combined extrusion method, in which the catheter tube is co-extruded together with the wire forming the spiral.

The individual turns 2028 of the spiral in this case need not necessarily be arranged precisely centrally inside the wall 2012, as is represented in FIG. 26, but may also be arranged more toward the outer side 2010 or toward the inner side 2011. The turns 2028 may also protrude somewhat from the outer side 2010 or the inner side 2011, so that a correspondingly uneven (undulating) surface is obtained. In one advantageous configuration, the outer diameter D2 of the turns 2028 is less than the outer diameter of the catheter tube 2022, and/or the inner diameter D1 of the turns 2028 is greater than the inner diameter of the catheter tube 2022.

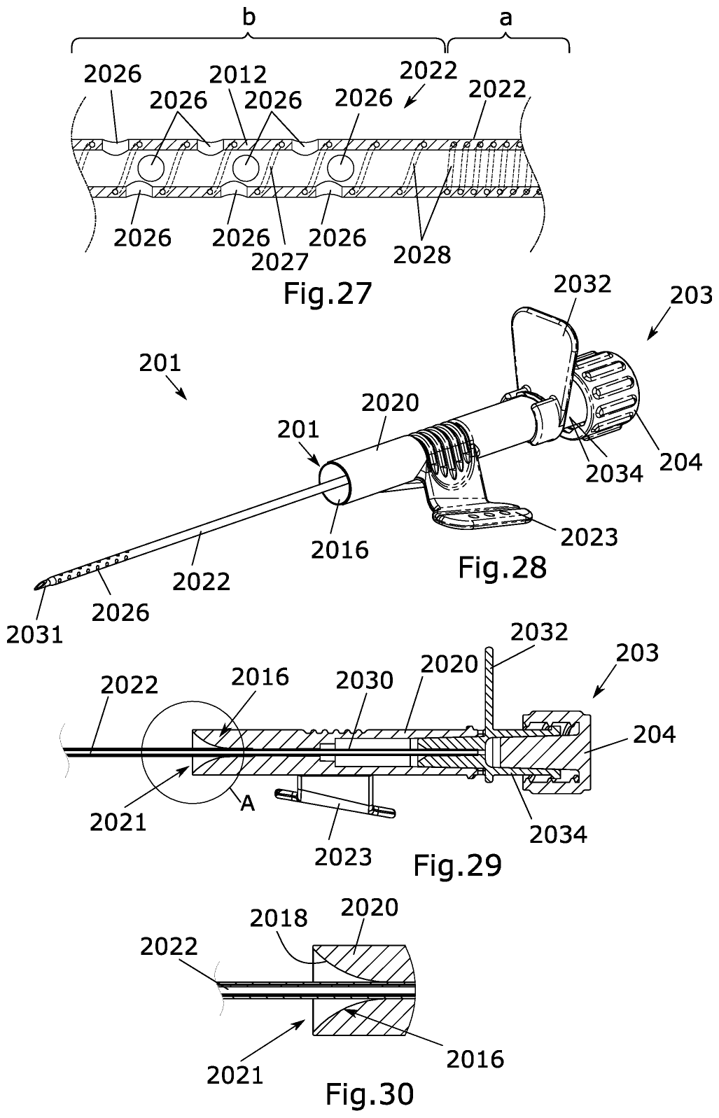

FIG. 27 shows a detail of the catheter tube 2022 in the transition region of the longitudinal section a into the longitudinal section b. The way in which the spiral supporting structure 2027 transitions from a small spacing of the turns 2028 into a larger spacing of the turns in the longitudinal section b may be seen. In the longitudinal section b, there are also the passage openings 2026, and it may be seen that the passage openings 2026 are arranged between the turns 2028.

FIG. 28 shows an indwelling cannula 201 in which a kink protection structure 2016 is formed on the housing 2020 in the tube exit region 2021. FIG. 28 shows the indwelling cannula 201 in a perspective view, FIG. 29 shows it in a lateral sectional view and FIG. 30 shows an enlarged representation of the region A marked in FIG. 29. The kink protection structure 2016 may, for example, be formed in an indentation 2018 radially enclosing the catheter tube 2022 with a wall having a convex curvature arched toward the catheter tube 2022, in a similar way to a sound exit side of a trumpet. The kink protection structure 2016 may have a rotationally symmetrical shape. The indentation 2018 may extend concentrically around the catheter tube 2022. If the catheter tube 2022 is now bent to the side in the region of the tube exit region 2021, the flexible catheter tube 2022 bears on the curved wall of the kink protection structure 2016 and is thus protected against kinking at least under moderate loads.

FIGS. 28 and 29 also show that the needle device 203 may have an attachment port 2034 for attaching a syringe or an infusion line at the proximal end, i.e. in the region of the handling element 2032, for example for blood aspiration. The attachment port 2034 is closed by a closure cap 204. The closure cap 204 may, for example, be screwed or plugged on.

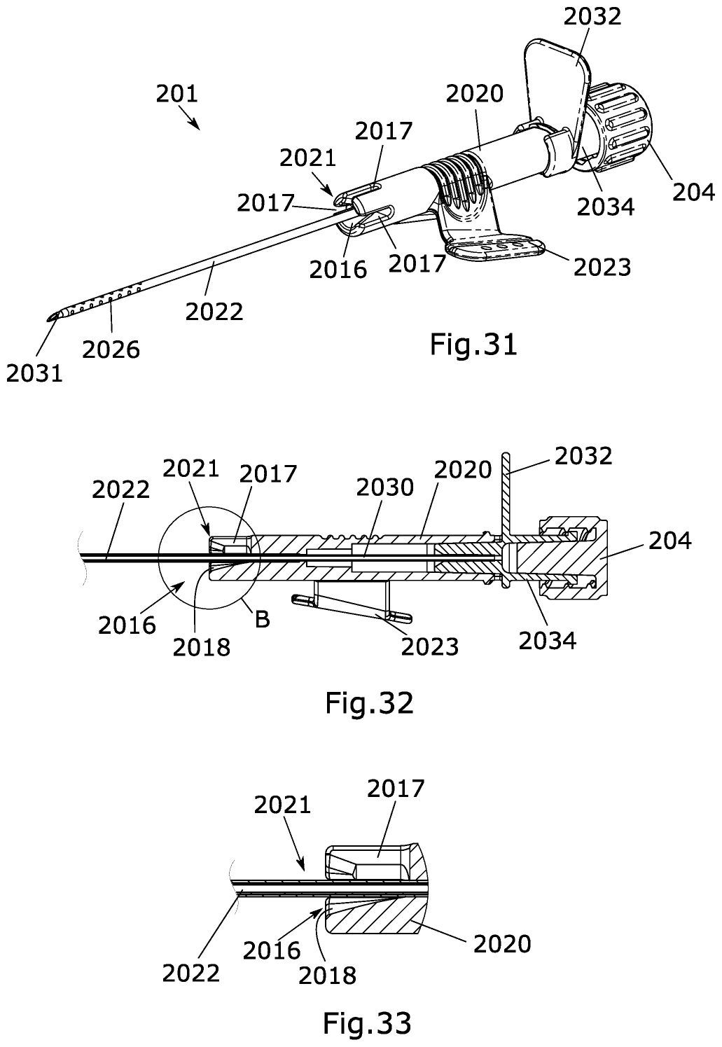

FIGS. 31 to 33 show an embodiment of an indwelling cannula 201 in which the kink protection structure 2016 is formed fundamentally similarly as in the embodiment of FIGS. 28 to 30, there being the differences explained below. FIG. 31 shows the indwelling cannula in a perspective view, FIG. 32 shows it in a lateral sectional representation and FIG. 33 shows an enlarged representation of the region B marked in FIG. 32.

First, it may be seen that the indentation 2018 is configured with a curved wall having convex curvature toward the catheter tube 2022, in which the curvature is less than in FIGS. 28 to 30. The indentation 2018 is configured with a more acute opening angle than in the embodiment of FIGS. 28 to 30. A plurality of, for example three, slot-shaped recesses 2017 extending in the longitudinal direction L are additionally formed into the housing 2020. The slot-shaped recesses 2017 begin at the tube exit region 2021 of the housing 2020 and extend by a particular amount, for example along the length of the indentation 2018, into the housing 2020. By the slot-shaped recesses 2017, the mobility of the catheter tube 2022 in relation to the housing 2020 is possible to an increased scope in particular directions defined by the slot-shaped recesses 2017.

A hinge may also be arranged in the transition region from the housing 2020 to the respective fixing wing 2023, so that the respective fixing wing 2023 is pivotably, i.e. tiltably, connected to the housing 2020 about the hinge. This applies for all exemplary embodiments of the invention.

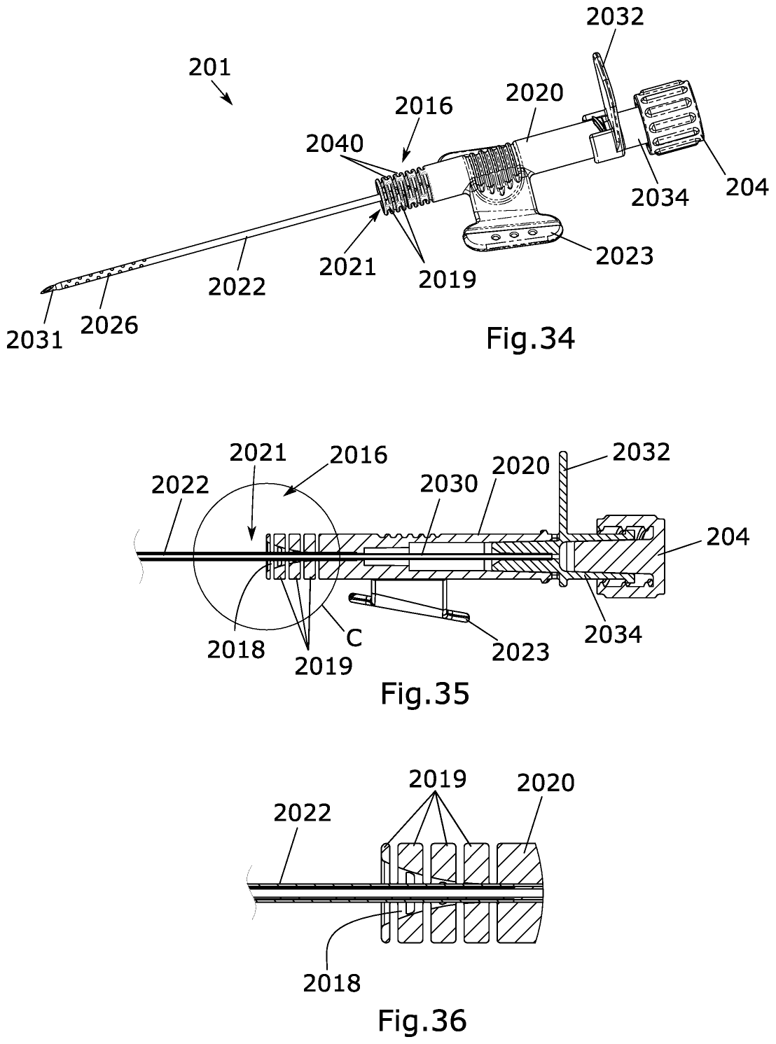

FIGS. 34 to 36 show an embodiment of an indwelling cannula 201 in which the kink protection structure 2016 itself has an increased flexibility or resilience in relation to the housing 2020. FIG. 34 shows the indwelling cannula 201 in a perspective view, FIG. 35 shows it in a lateral sectional view and FIG. 36 shows an enlarged representation of the region C marked in FIG. 35.

For example, the kink protection structure 2016 may be configured in a similar way as in the case of kink protection for electrical lines which emerge from a housing of an electrical apparatus. For example, the flexible kink protection structure 2016 may be formed from a plurality of rings 2019 arranged around the catheter tube 2022 successively in the longitudinal direction, which are connected to one another and to the housing 2020 by connecting webs 2040. For example, the housing 2020 may be connected to the first ring 2019 by means of two connecting webs 2040 arranged on opposite sides. The first ring 2019 may be connected to the second ring 2019 by means of two connecting webs 2040 arranged on opposite sides, in which case the connecting webs 2040 between the first and the second ring 2019 may be arranged offset in the circumferential direction with respect to the connecting webs 2040 between the housing 2020 and the first ring 2019 by a particular angle measurement, for example 90 degrees. In the same way, the further rings 2019 may be connected to the respectively neighboring ring 2019 by means of two respective connecting webs 2040, in which case the connecting webs may again be offset alternately in the circumferential direction by a particular angle measurement, for example 90 degrees. The kink protection structure 2016 formed in this way may be configured relatively flexibly, for example consisting of an elastomer material, for example rubber.

As also shown by FIGS. 35 and 36, an indentation 2018 of the type described above, for example in a trumpet shape, may also be formed inside the kink protection structure 2016 formed by the rings 2019 with the connecting webs 2040.

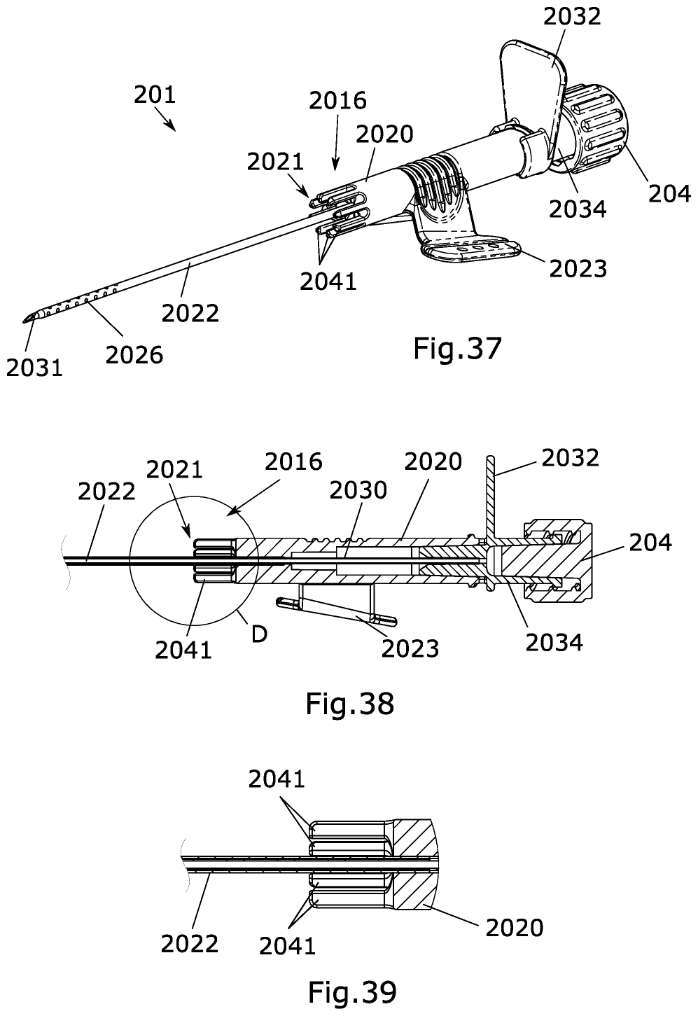

FIGS. 37 to 39 show an embodiment of an indwelling cannula 201 in which the kink protection structure 2016 is formed by a multiplicity of pins 2041 projecting from the housing 2020 in the longitudinal direction toward the distal end of the indwelling cannula 201. FIG. 37 shows the indwelling cannula 201 in a perspective view, FIG. 38 shows it in a lateral sectional view and FIG. 39 shows an enlarged representation of the region D marked in FIG. 38.

The pins 2041 are respectively arranged with a certain spacing from one another, which is great enough that the catheter tube 2022 can extend into such an intermediate space in the event of a bending load. In addition, the pins 2041 may be spaced apart from the catheter tube 2022 in the radial direction, i.e. they may form a certain free space around the catheter tube 2022 in order to allow freer mobility of the catheter tube 2022 in the event of bending loads.

In general, in all embodiments described the kink protection structure 2016 has the advantage that an increased bending radius of the catheter tube 2022 is ensured in the tube exit region 2021, and possible kinking of the catheter tube 2022 can thereby be counteracted in this region.

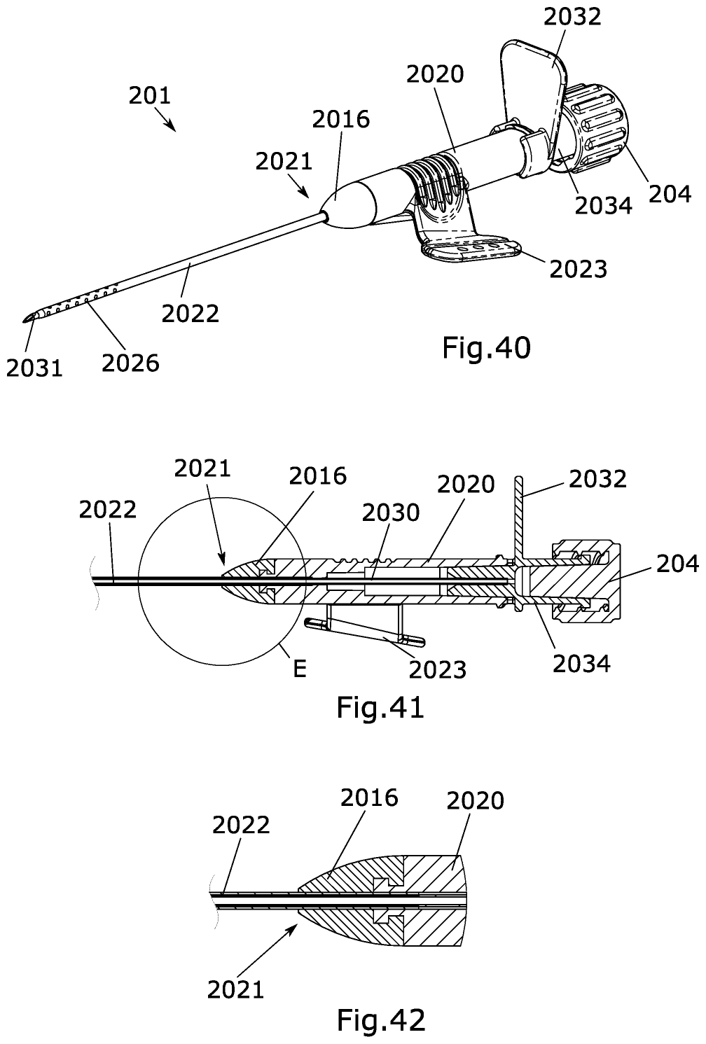

The kink protection structure may have an increased resilience in relation to the resilience of the housing 2020 even if, for example, it does not have the slot-shaped recesses 2017 or the pins 2041 but is configured comparatively solidly. Kinking of the catheter tube 2022 in the tube exit region 2021 is also thereby counteracted. FIGS. 40 to 42 show an embodiment of an indwelling cannula 201 in which such a kink protection structure 2016 is employed. FIG. 40 shows the indwelling cannula 201 in a perspective view, FIG. 41 shows it in a lateral sectional view and FIG. 42 shows an enlarged representation of the region E marked in FIG. 41.

The kink protection structure 2016 may be configured as a constituent part separate from the housing 2020, and may advantageously consist of a material having a higher resilience than the material of the housing 2020, for example from an elastomer material or a silicone material. FIGS. 40 to 42 show an embodiment of such a kink protection structure 2016 which is solidly configured. It is also possible for such a kink protection structure 2016 consisting of a more resilient material to have one or more of the features of the kink protection structure 2016 which have been mentioned above, for example the indentation 2018.

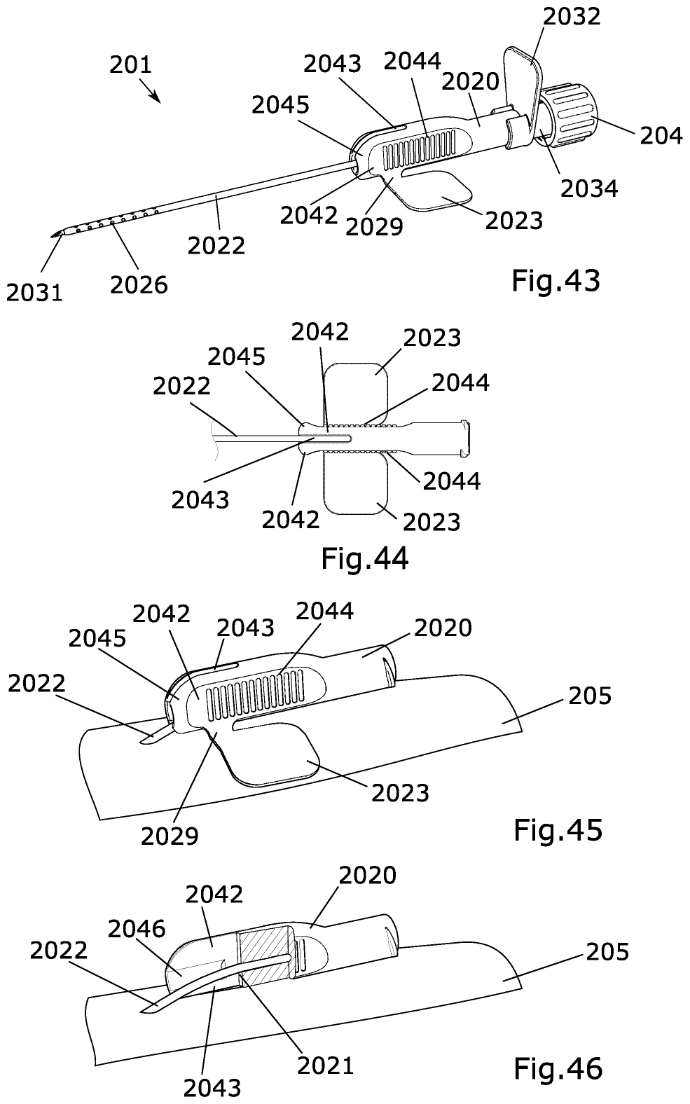

FIGS. 43 to 46 show an embodiment of an indwelling cannula 201 in which, in order to form a grip region, grip faces 2044 for manual gripping and manipulation of the indwelling cannula 201 are respectively present on the left and right on the housing 2020. FIG. 43 shows a perspective view of the indwelling cannula 201, FIG. 44 shows the proximal part of the indwelling cannula 201 in a plan view, FIG. 45 shows the indwelling cannula 201 in the state applied on the patient and FIG. 46 shows a partially sectional representation of the indwelling cannula 201 in the state applied on the patient.

The grip faces 2044 may, for example, have ribs in the transverse direction. The housing 2020 also has two protecting projections 2042 extending on the left and right away from the catheter tube 2022 in the distal direction. As illustrated in FIG. 46, the protecting projections 2042 protrude from the housing 2020 on the tube exit region 2021. Guide slots 2043 are respectively arranged above and below the catheter tube 2022 between the protecting projections 2042. In particular, the lower guide slot 2043 makes it possible that the catheter tube 2022 in the state installed on the patient 205 can extend in a smooth bend toward the patient 205, i.e. the guide slot 2043 provides a space for defined bending of the catheter tube 2022 and prevents kinking or breaking of the catheter tube 2022. The catheter tube 2022 is additionally protected laterally in this region by the protecting projections 2042. A protective cap receptacle 2046 may be formed on the protecting projections 2042, for example in the form of a recess extending into the protecting projections 2042. In this way, a protective cap may be fitted over the puncture needle tip 2031 and the catheter tube 2022 and be fastened on the housing 2020.

The grip faces 2044 may extend toward the distal end into the region of the protecting projections 2042. Advantageously, a finger stop device 2045 is arranged at the distal end of the housing 2020, or of the protecting projections 2042, for example in the form of a thickening, for example a bead or a flange. This prevents the user from slipping with their fingers forward toward the patient 205 from the housing 2020, or from the grip faces 2044, for example when installing the indwelling cannula on the patient 205.

It may furthermore be seen that, in the embodiment of FIGS. 43 to 46, the fixing wings 2023 are connected to the housing 2020 only by a relatively narrow material bridge 2029. Accordingly, the fixing wings 2023 extend freely from the housing 2020 over a certain length region so that an intermediate space from the housing 2020 is formed there, through which for example an adhesive strip may be passed in order to fasten the fixing wings 2023 on the patient 205.

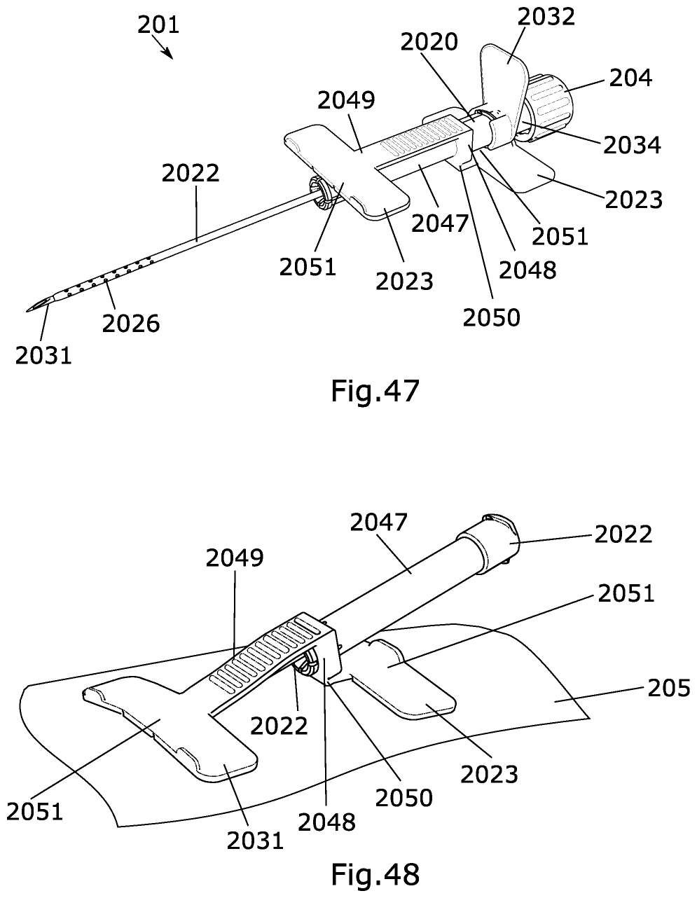

FIGS. 47 and 48 show an embodiment of a catheter in which the fixing wings 2023 are arranged movably, i.e. linearly displaceably, in the longitudinal direction L in relation to the housing 2020. FIG. 47 shows the indwelling cannula in the basic state, i.e. in the delivery state. FIG. 48 shows the indwelling cannula 201 without the needle device 203 after installation on the patient 205. FIGS. 47 and 48 respectively represent perspective views.

The fixing wings 2023 are located on the one hand on a rear wing carrier 2051, which in the basic state represented in FIG. 47 is located near to the proximal end of the indwelling cannula 201, and on a front wing carrier 2051, which is spaced apart from the rear wing carrier 2051 in the longitudinal direction L and accordingly is arranged nearer to the distal end of the indwelling cannula 201. The rear wing carrier 2051 is connected by means of an articulated connection 2050 to a sliding sleeve 2048. The sliding sleeve 2048 is connected by means of a longitudinal connector 2049 to the front wing carrier 2051. In this way, all the fixing wings 2023 are connected to one another so as to form a unit.

The housing 2020 has a sliding section 2047, which is used for longitudinal displacement of the sliding sleeve 2048 on this sliding section 2047. The sliding section 2047 therefore has a constant cross-sectional contour and cross-sectional size over its longitudinal extent. FIGS. 47 and 48 show a sliding section in a cylindrical shape, although the sliding section may also have other shapes, for example a polygonal shape.

In the basic state represented in FIG. 47, the indwelling cannula 201 is installed in the usual way on the patient 205. If the indwelling cannula 201 is then intended to be fixed on the patient 205, the unit which is displaceable by means of the sliding sleeve 2048, and on which all the fixing wings 2023 are located, is pushed forward along the sliding section 2047 from the proximal end until the position represented in FIG. 48 is reached. In this position, the indwelling cannula 201 may be fastened on the patient 205 by the fixing wings on the front wing carrier and the rear wing carrier 2051 respectively being fastened thereon with an adhesive strip.

The front wing carrier 2051 is arranged above the housing 2020 in the basic state, while the rear wing carrier 2051 is arranged below the housing 2020, respectively as seen from the patient application side. In the state applied on the patient 205, the two wing carriers 2051 are located directly on the patient 205, i.e. at the same height level. The catheter 202 is thereby put into a suitable oblique setting by which the catheter tube 2022 is protected against unnecessary bending in the tube exit region 2021. It may also be seen that, in this position, the longitudinal connector 2049 forms an advantageous protective cover for the section of the catheter tube 2022 located outside the patient 205.

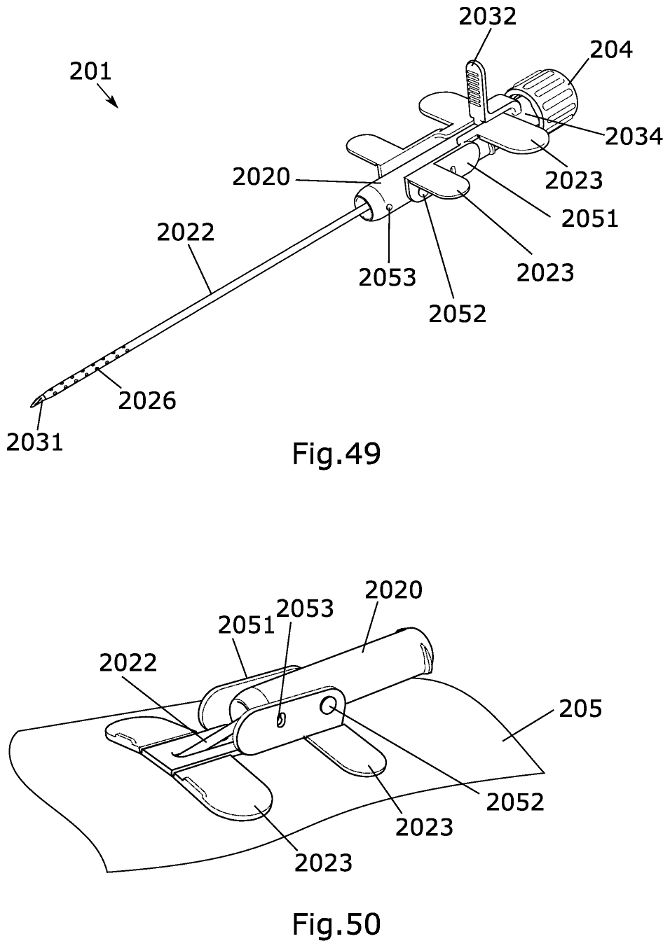

In views similar to FIGS. 47 and 48, FIGS. 49 and 50 show an indwelling cannula 201 with an alternatively configured mobile device for readjusting the position of the fixing wings 2023. There is one wing carrier 2051 in this embodiment, which is formed in the manner of two bodies extending laterally along the housing 2020, on which two fixing wings 2023 are arranged respectively on the front and rear end. The wing carrier 2051 is fastened pivotably about a pivot axis 2052 on the housing 2020. FIG. 49 shows the indwelling cannula 201 in the basic state. In this state, the free pivotability of the wing carrier 2051 can be prevented by a holding element so that the wing carrier 2051 does not undesirably change its position during the application of the indwelling cannula 201 on the patient. For example, the handling element 2032, which may be connected by means of a flexible web to the housing 2020 and in this way can be manually deflected in order to release the fixing of the wing carrier 2051, may be used as the holding element.

If, when the indwelling cannula 201 is installed on the patient 205 and is intended to be fastened thereon, the wing carrier 2051 is pivoted into the position represented in FIG. 2050, the fixing wings 2023 bear on the patient 205 and may be fastened thereon by means of adhesive strips. In this pivoted position, the wing carrier 2051 may be fixed by means of latching using at least one fixing pin 2053 engaging into a recess. In this way, the housing 2020 is locked in a particular pivoted position relative to the wing carrier 2051.

In all exemplary embodiments of the invention, the handling element 2032 may have structuring, for example in the form of ribs, on the side directed toward the patient and/or on the side turned from the patient. The fixing wings 2023 may be configured in such a way that they are pliable or moldable, so that they may be readily adapted by the user to the corresponding surface of the body region of the patient, on which the indwelling cannula 201 is arranged.

### 3RD EMBODIMENT

### Puncture Device

The invention relates to a puncture device for puncturing a hollow body by means of a puncture needle, wherein the puncture device has at least one vasiform outer tunic, in which the puncture needle can be guided longitudinally displaceably, wherein the outer tunic is adapted to be pushed over at least a part of its length through the opening created by means of the puncture needle through the tunic of the hollow body to be punctured after puncture of a tunic of the hollow body to be punctured has been carried out, and to remain there over a period of time.

Such a puncture device will also be referred to below as a puncture system or, particularly when it relates to the medical field, as an indwelling venous cannula. The vasiform outer tunic, or “outer tunic” for brevity, will also be referred to below as a venous catheter. The hollow body to be punctured may be a hollow body of a living being or a hollow body of an object. If the puncture device relates to the medical field, the hollow body may for example be a blood vessel, for example a vein or artery.

The terms “puncturing” and “puncture” are in this case to be understood in the medical sense. Puncturing refers to the entry of a puncture needle into the hollow body, in such a way that the puncture needle penetrates through the tunic of the hollow body to be punctured.

Those parts which are arranged at the remote (distal) end of the puncture device, from the viewpoint of the user, and therefore in the vicinity of the punctured hollow body, are regarded as a section of parts of the puncture device near to the hollow body. Accordingly, sections remote from the hollow body are arranged at the proximal end of the puncture device from the user's viewpoint, i.e. further away from the punctured hollow body. In connection with medical applications of the puncture device, the terms near-vein or near-patient are also used synonymously with the term near to the hollow body, and vein-remote and patient-remote are also used synonymously for the term remote from the hollow body.

A piercing-resistant indwelling venous cannula is disclosed by PCT/EP2019/057097.

An improved puncture system in the sense of a general puncture system is intended to be provided. In principle, with the improved indwelling venous cannula it is likewise possible advantageously to puncture all body cavities and interstitial body spaces and all anatomical and morbid structures which are intended to be punctured, and to install a catheter in them.

Components of the improved indwelling venous cannula may in principle also be combined with all known puncture and catheter systems or be used as independent products.

Although the term “indwelling venous cannula” will be adopted below, it has a wider meaning in the sense of a general puncture system with which not only veins may be punctured. In what follows, the terms “vein” and “veins” thus include in principle all blood vessels and very generally all body cavities and interstitial body spaces and all anatomical and morbid structures which are intended to be punctured and provided with a catheter.

Thus, in addition, by way of example the trachea, pleural space, abdomen, stomach, intestine, renal pelvis, urinary bladder, cerebrospinal fluid space and bone may be punctured with the indwelling venous cannula. In addition, morbid structures such as abscesses in and on the patient may be punctured. Advantageously, arterial blood vessels may also be punctured.

In what follows, the term “patient” includes all living beings of all age groups and genders. Applications in technical fields and in and on all objects and structures, for example in and on reservoirs, containers, cavities, extensible materials and in or on pump, hose, pipe and port systems, are likewise explicitly possible.

When the word “or” is used in what follows, possible alternatives are indicated but combinations of the features or configurations separated by “or” are also fundamentally explicitly possible.

In what follows, “near-vein” may usually be equated with “patient-near”, and “vein-remote” with “near-user”. Near-vein components tend to be located inside the patient, and vein-remote components tend to be arranged outside the patient. This need not necessarily always apply, but is meant to further clarify the terms. The additional elements are generally self-evident, and the context is essential.

All components described may be employed singly or multiply on an indwelling venous cannula or also, independently of the latter, on/in other products or entirely independently. Various features of different components are also freely combinable, and features of particular components may also be used in other components without this being explicitly mentioned. All components and features may fundamentally be employed inside and outside a patient.

### Special Cross-Sectional Areas of the Venous Catheter

According to one advantageous configuration of the invention, it is provided that the outer tunic and/or another part of the puncture device has one or more flow channels running predominantly or fully in the longitudinal direction of the puncture device, through which a fluid flowing through the punctured hollow body can flow along past the outer tunic and/or the other part of the puncture device. Such a flow channel may, for example, be formed by a trench- or furrow-shaped indentation, for example a groove, on the outer side of the outer tunic or a channel in the wall of the outer tunic. The flow channel may also be configured so as to vary over its length, partially as an indentation and partially as a channel in the wall of the outer tunic. The flow channel need not be present over the entire length of the outer tunic. For example, the flow channel may be arranged exclusively or predominantly in the section of the outer tunic adapted to remain in the hollow body. This applies for one, several or all of the flow channels. The flow channels of the outer tunic may, in particular, run predominantly or fully in the longitudinal direction of the outer tunic.

The cross-sectional area of the venous catheter may be a circle or an ellipse. One side of this circle or ellipse may have at least one flattening, indentation or protuberance. The venous catheter may thereby also be adapted optimally to veins that do not have a circular cross-sectional area. Over a partial length or its predominant or entire length, the venous catheter may consist as a whole or partially of a thermoplastic material, for example a plastic material, which has the property that it no longer maintains a special shape of the venous catheter at body temperature, and the venous catheter can therefore be adapted optimally to the shape of the vein. The wall of the venous catheter may therefore bear against the vein wall. The venous catheter may therefore keep the lumen of the vein open for a flow of fluid through the venous catheter when such a fluid is applied through the venous catheter. If such a fluid is no longer being applied through the venous catheter, the venous catheter collapses because of the pronounced thermoplastic properties and therefore closes itself. This has the advantage that fluid, for example blood, can then no longer inadvertently emerge from the patient through the venous catheter.

The venous catheter may likewise have at least one trench-/furrow-shaped indentation on its outer side. This indentation may, for example, be a longitudinally running indentation. A flow of fluid may therefore be achieved next to the venous catheter even when the venous catheter is in the vein. When the venous catheter is installed, flow perturbations in the vein may for example thereby be prevented. In the field of arterial vessel puncture, this may be of particular relevance for avoiding blood circulation perturbations. This is likewise the case if the actual lumen of the venous catheter were to be obstructed or clogged, for example by coagulated blood. In the region of the indentation, the venous catheter may as a whole or partially consist of at least one material which has antimicrobial and/or anticoagulant properties, or be coated with such a material. The accumulation of germs and/or blood clots is therefore prevented in this region. Likewise, at least one material which has particularly favorable fluid guiding properties and does not significantly damage the corpuscular constituents of the blood upon blood contact may be used in this region.

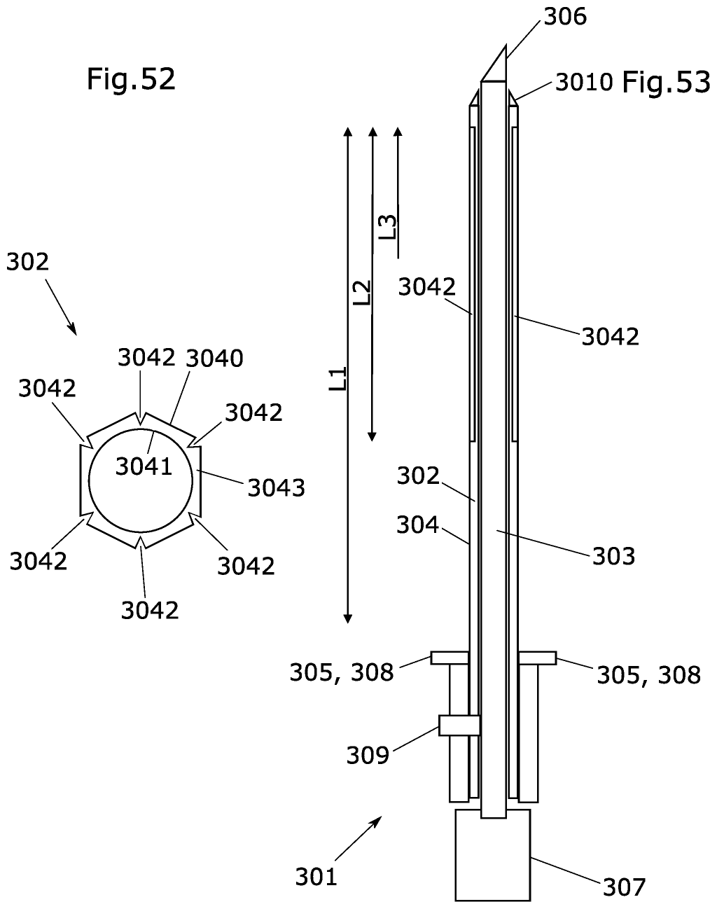

The indentation need not exist over the entire length of the venous catheter. For instance, the venous catheter may advantageously not be formed remotely from the vein in the region of the transition from the venous catheter into the other components of the indwelling venous cannula and/or where the venous catheter leads through the skin and the subcutaneous tissue of the patient. There are therefore no entry ways for germs in this region, since the skin and subcutaneous tissue can lie completely around the venous catheter. Likewise, the indentation may already end before the near-vein tip of the venous catheter, so that it can be formed without edges (FIGS. 52, 53). The indentation may also be formed by the venous catheter consisting at least partially, for example on its outer side, of at least one resorbable material that has the complementary shape of the indentation and initially at least partially fills the indentation. If this material is then resorbed in the patient, for example upon blood contact or upon contact with other fluids, the indentation is formed. This resorbable material may be a carbohydrate, or alternatively another biomolecule or a salt. This material may also be or contain magnesium. Resorbable polymers, composites, bioceramic materials or biodegradable metals may likewise be employed. The material may also be a combination of a plurality of resorbable materials.

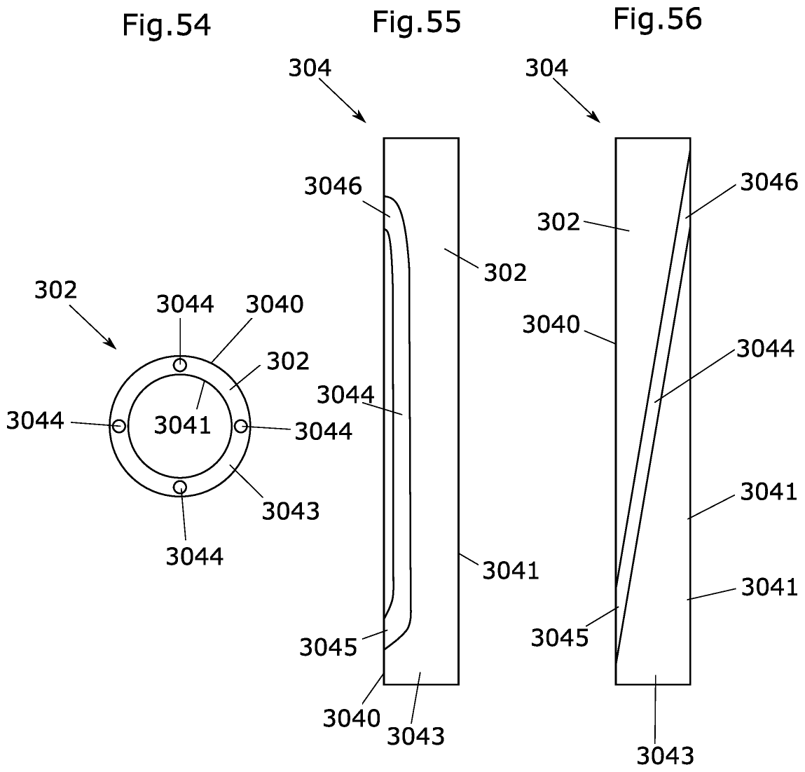

It is also conceivable that, instead of the indentation, there is at least one longitudinally running channel in the wall of the venous catheter, which is delimited by the actual lumen of the venous catheter. This channel may begin remotely from the vein via a recess in the wall of the venous catheter and end near to the vein via a recess which is located directly in the region of the tip of the venous catheter, or somewhat away therefrom in the wall of the venous catheter (FIGS. 54 to 56). At least one additional flow of fluid is thereby made possible in the venous catheter. Combinations of the aforementioned trench-/furrow-shaped indentations and channels are possible.

An expansion body for fixing the location of the venous catheter in the punctured body part may be arranged on the venous catheter of the indwelling venous cannula. Such an expansion body may for example be configured as an inflatable cuff which, for example, is arranged on the outer side of the venous catheter. In one advantageous development, this expansion body may then likewise have at least one trench-/furrow-shaped indentation as described above, which is preserved even in the expanded state in order to allow a flow of fluid around the expansion body. It is also conceivable that the expansion body contains at least one channel as described above even in the expanded state, so that a flow of fluid is possible through the expansion body. This channel may be kept open by a circular structure which at least partially encloses it and forms the wall.

A venous catheter as described above may also be used independently of an indwelling venous cannula, for example in a longer embodiment with a larger diameter as a hose of a device for carrying out extracorporeal membrane oxygenation (ECMO).

The cross-sectional area of the venous catheter may also be configured in a similar way to an externally serrated gearwheel having at least one tooth. The gearwheel may in this case have 2, 3, 4, 5, 6, 7, 8, 9, 10, 11, 12, 13, 14, 15, 16, 17, 18, 19, 20, 21, 22, 23, 24, 25, 26, 27, 28, 29, 30 or more than 30 teeth. As an alternative or in addition, the cross-sectional area of the venous catheter may however also have an internally serrated gearwheel having a number of teeth as just mentioned. It is also conceivable for at least one further lumen located in the venous catheter to have external serration and for the venous catheter itself to have internal serration.

Configuration of the cross-sectional area of the venous catheter as a semicircle, semiellipse, arc or ring is likewise possible, as well as at least one flattening, indentation or protuberance on each side of these geometrical figures/shapes (referred to below only as “shapes”). There may also be trench-/furrow-like shapes. Likewise possible is configuration of the inner and/or outer cross-sectional area as a rectangle, square, cross, parallelogram, rhombus, triangle, pentagon, hexagon, octagon or very generally a polygon. Shapes resembling a frame, cloverleaf or half-moon may also be envisioned.

The venous catheter may have the shape of a cylinder, cone, cuboid or also prismatic shapes. A pyramidal configuration is also possible. The venous catheter may likewise have a wing profile, more precisely an airfoil wing profile.

It is possible that both the inner and outer side of the venous catheter respectively have at least one of the aforementioned shapes, or are developed with structures that have at least one of the aforementioned shapes. The outer and inner sides of the venous catheter may also differ in these properties. For example, it is conceivable for the outer side of the venous catheter to be flexibly adapted to the shape of the vein but for the inner side of the venous catheter to have more rigid properties, which keep the lumen of the venous catheter open against the effect of external pressure. These rigid properties may be achieved by a special shape of the inner side of the venous catheter, for example by an oval shape, but also by the use of special materials, in particular metals, metal alloys and hard plastic materials. These rigid properties may also be provided by a spiral, undulating or network-shaped surface of the inner side of the venous catheter. A combination with structures running transversely, longitudinally or diagonally may also be envisioned, and these may also ensure the required rigidity on their own.

All geometrical shapes mentioned, and further geometrical shapes, may be freely combined. Different sections of the venous catheter may also have different cross-sectional areas or shapes.

By special properties, for example thermoplastic properties, these may change when the venous catheter is exposed to different ambient conditions, for example the body temperature. They may also change when the venous catheter is exposed to a certain pressure from the inside or outside. It is therefore conceivable, for example, to create a universal blood vessel catheter which may be used both under conditions of low blood pressure, for example in veins, but also conditions of high blood pressure, for example in arteries or near to the heart.

The combination of different shapes may also be used in order to delimit a plurality of lumens in the venous catheter from one another. For example, the venous catheter may have at least one lumen that has a circular cross-sectional area, and another that has an ellipsoidal or rectangular cross-sectional area. The cross-sectional area may respectively be optimized for special properties of different fluids.

### Barb-Like Structures

According to one advantageous configuration of the invention, it is provided that barb-like structures, which impede or prevent extraction of the puncture device inserted into the hollow body, are present on the outer side of the outer tunic. The puncture device is thereby secured against accidental sliding out of the punctured hollow body.

Structures which constitute or resemble barbs may also be fitted on the outer side of the venous catheter. These may for example be configured to be, and be, capable of being folded out or pulled outward. By way of example, they may be activatable by a flow of fluid, gas or vapor. By way of example, they may also be activatable by a blood flow, which may be pulsatile or non-pulsatile/continuous.

These structures may therefore impede or prevent extraction or slipping of the venous catheter out of the vein. The venous catheter may also be configured in a similar way to a dowel. It is conceivable that structures located on the outer side, for example elongate structures, move away from the outer side of the venous catheter, for example by jutting from the venous catheter, when warmed to body temperature. These structures may also experience a volume increase when warmed to body temperature. Likewise, these structures may also be configured to absorb fluid and thereby experience a volume increase upon contact with fluids, for example blood, vapors or gases. The venous catheter itself may thereby stabilize its location in the vein.

The venous catheter may also be configured as a telescopic tube, for example a circular telescopic tube, and be readjustable in length. It may in this case consist of 2, 3, 4, 5, 6, 7, 8, 9, 10 or more than 10 elements displaceable in one another, for example rings, having different inner and outer diameters.

What has been stated in this section may likewise apply generally for puncture needles and for catheters and hoses. These may have the aforementioned properties and advantageously be developed as just described.

Special Construction of the Venous Catheter with an Inner-Lying Capillary System

According to one advantageous configuration of the invention, it is provided that the outer tunic has an arrangement of a multiplicity of elongate cavities that extend at least predominantly in the longitudinal direction of the outer tunic, in which case the cavities may be configured particularly in the form of capillaries.

The venous catheter may also be formed from a system of elongate cavities, for example capillaries, which may be configured with very thin walls. The capillaries may also be configured as glass capillaries. These may for example have an outer diameter of between 0.01 and 2.5 millimeters, preferentially between 0.05 and 0.5 millimeters. Smaller or larger outer diameters may, however, also be envisioned. The wall thickness/wall width (in what follows, only “wall thickness”) may for example lie between 0.002 and 0.25 millimeters, preferentially between 0.01 and 0.05 millimeters. Smaller or larger wall thicknesses may, however, also be envisioned.

Capillary forces may advantageously act in a venous catheter configured in this way. Advantageously, for example, a venous catheter configured in this way may also be wetted with further fluids or substances, for example anticoagulant or antimicrobial substances.

The elongate cavities described above may also be formed from other materials, for example plastic materials or metals or metal alloys. They may be electrically conductive and, for example, also act as sensors. Heating or cooling of a fluid flowing through may be envisioned; co-current and counter-current flow principles may be employed. The walls of the elongate cavities may be permeable only for particular substances over their partial length or over their predominant or entire length. For example, they may be semipermeable or allow other diffusion processes. It is also conceivable that fluids flowing in the venous catheter are rid of harmful substances and therefore, for example, detoxification takes place.

### At Least Three-Layer Construction of the Indwelling Venous Cannula

According to one advantageous configuration of the invention, it is provided that the puncture device has a further vasiform structure, which is arranged between the outer tunic and the puncture needle.

An at least three-layer construction of the indwelling venous cannula is therefore possible. Between the inner-lying puncture needle and the outer-lying venous catheter, there may be at least one further vasiform/cylindrical structure, which may also be configured as a hose or catheter. This central structure may also have another shape and, for example, have the cross-sectional areas mentioned above. The central structure is arranged longitudinally displaceably in relation to an inner-lying puncture needle and/or in relation to the outer-lying venous catheter, and/or twistably about its own longitudinal axis.

The wall of this central structure may be formed from at least one layer and partially, predominantly or fully enclose the inner-lying puncture needle or yet another inner-lying catheter. The wall of the central structure may preferentially be formed from at least two or three layers, at least one of which has at least partially piercing-resistant properties. The layer having these properties may for example consist at least partially of a metal, a metal alloy or of aramid/aramid fibers. The central structure may thus provide piercing resistance in relation to the tip of the puncture needle for at least one outer-lying catheter.

The central structure may also be configured in the form of a semicircle or bowl. In such a version, it encloses the inner-lying puncture needle only partially. In one preferred version, it encloses the surface of the puncture needle over a part or its entire length in the form of a semicircle or bowl to at least 5% or 10%, in further preferred versions to at least 20%, 30%, 40%, 50%, 60%, 70%, 80% or 90%.

Besides an embodiment having a smooth or pimpled surface, the central structure may also be configured spirally and provide an undulating surface. It may also be provided with a sealing coating, in particular a PTFE coating, and may additionally be reinforced with further structures or elements, that is say for example by transversely, longitudinally or diagonally running structures.

In comparison with an outer-lying venous catheter, the central structure may have a significantly reduced wall thickness. For instance, the wall thickness may be less than 90%, in advantageous versions even less than 80%, 70%, 60%, 50%, 40%, 30%, 20%, 10% or 5%.

The central structure may also be configured as a conventional guide wire, for example without a core, through which the puncture needle is internally guided longitudinally displaceably. In such a case, the outer-lying venous catheter may also be obviated. This results in a system consisting of an inner-lying puncture needle and an outer-lying guide wire, which is used to place a guide wire in a target structure, for example in a vein.

### Lotus Effect

According to one advantageous configuration of the invention, it is provided that the outer side of the puncture needle and/or the inner side of the outer tunic has a surface layer which is formed from a material having an increased slideability with respect to the other material of the puncture needle and/or of the outer tunic. For example, a kind of lotus effect may thereby be provided on the outer side of the puncture needle and/or the inner side of the outer tunic.

The outer side of the puncture needle may consist of a particularly slideable material or be coated with such a material. As an alternative or in addition, the inner side of the venous catheter may consist of such a material or be coated with such a material. This may, in particular, be a material, a coating and/or surface structures which induce a lotus effect on their own or in combination. Hydrophobic properties up to the extent of superhydrophobicity may thereby be achieved. Corresponding surface structures may in particular have pimples, and at least one wax layer may advantageously be used. Self-cleaning properties may thereby be imparted to the corresponding surfaces of the puncture needle and of the venous catheter. All other components and surfaces of the venous catheter may also be configured in the manner just mentioned. Advantageous effects on the flow resistance in the interior of the venous catheter may be envisioned.

### Friction-Reducing Properties Between the Puncture Needle and the Venous Catheter

According to one advantageous configuration of the invention, it is provided that there is a friction-reducing auxiliary substance, in particular a substance containing oil or fat, on the outer side of the puncture needle and/or the inner side of the outer tunic.

It is also possible that at least one substance containing oil, fat or the like is located between the puncture needle and the venous catheter, which reduces the frictional resistance between the two. It may also have antimicrobial or anticoagulant properties. So that this substance remains in the space between the puncture needle and the venous catheter, this space may advantageously be developed near to the vein and remotely from the vein, respectively, with a bounding element. This bounding element may by way of example be configured so that it encompasses the puncture needle partially or fully, for example circularly, and the latter is guided through a recess of the bounding element longitudinally displaceably and/or twistably about the longitudinal axis.

It is also conceivable that at least one bounding element is mounted longitudinally displaceably in the venous catheter. It may be connected to an actuation element by which the location of the bounding element can be adjusted by a user. If the puncture needle then has a structure or a shape which increases the outer diameter of the puncture needle at least over a partial length thereof, the puncture needle can no longer pass through the bounding element over this partial length. The length over which the puncture needle tip projects beyond the venous catheter near to the vein may thus be adjusted variably within particular limits.

A bounding element as described above may form an undulating surface and/or also be configured in the form of a spring and/or spiral. A bounding element as described above may consist of a plastic, or also of a metal or a metal alloy. So that the bounding element is configured impermeably to gas, vapor and/or fluid, it may also be coated or clad with at least one further material. Here, use of PTFE may be envisioned.

### Hollow Guide Wire as a Venous Catheter

According to one advantageous configuration of the invention, it is provided that at least the part of the outer tunic which is adapted to remain in the hollow body has a spirally wound internally hollow guide wire over its entire length or over the predominant part of its length in which the puncture needle can be guided longitudinally displaceably. There is therefore a cavity inside the spiral structure, through which for example a puncture needle may be guided.

It is further proposed that at least the part of the venous catheter which is adapted to remain in the patient consists over its entire length or the predominant part of its length, in which the puncture needle can be guided longitudinally displaceably, of a guide wire from which the inner core has been removed. It is therefore configured to be internally hollow and consists of a spiral structure. The latter may also be configured as a flat wire. This spiral structure may be covered with a gas-, vapor- and/or fluid-impermeable layer internally, externally or over its entirety. This layer may, for example, be a PTFE layer or another material.

### Puncture Needle (Tip) Consisting of Resorbable Material and Having Other Properties

According to one advantageous configuration of the invention, it is provided that the section of the puncture needle near to the hollow body is configured bluntly and/or consists of a bioresorbable material and/or any region of the puncture needle consists of a bioresorbable material or is coated with a bioresorbable material and/or at least a part of the outer tunic consists of a bioresorbable material or is coated with a bioresorbable material. Such a bioresorbable material has the advantage that it is dissolved in the body of a living being by the substances present there after a certain time. When resorbable materials are referred to below, this also includes bioresorbable materials.

It is therefore possible that the near-vein section of the puncture needle is configured not sharply but bluntly. It may by way of example also be configured in the form of a punch. If it is configured bluntly, this enables further applications in solid or even softer tissues, for example the brain, where a sharp puncture needle is unnecessary for penetrating the tissue and could cause injuries thereto.

The puncture needle may fundamentally be configured to be internally hollow or non-hollow. If it is configured to be non-hollow, detachment of the tissue and therefore possible drawing of tissue into more deeply lying anatomical structures may be avoided. The puncture needle may in this case be configured with an inner-lying core or further reinforcing but also flexible structures or materials. It may also contain an inner-lying mandrel, which is itself configured to be internally hollow or non-hollow. Preferentially, the near-vein tip of the puncture needle may also consist of a resorbable/dissolvable (in what follows, only “resorbable”) material or be coated with such a material.

This resorbable material may be a carbohydrate, or alternatively another biomolecule or a salt. This material may also be or contain magnesium. Resorbable polymers, composites, bioceramic materials or biodegradable metals may likewise be employed. The material may also be a combination of a plurality of resorbable materials, and also contain at least one antimicrobial or anticoagulant or otherwise effective or biologically active substance, or be coated with at least one such substance.

Upon blood contact or upon contact with other fluids, the shape of the puncture needle tip may therefore be altered and, for example, the near-vein tip of the puncture needle may be partially or wholly resorbed. Depending on the material used, this process may take place very rapidly, for example within 1 second, within 30 seconds or within 1 minute or 2 or 5 minutes. This process may however take place more slowly, for example taking at least 1 hour, at least 1 day, at least 1 week, at least 1 month, at least 3 or 6 months or at least 1 year.

It is also conceivable for the near-vein tip of the puncture needle only partially to consist or be coated with at least one resorbable material. Upon blood contact or upon contact with other fluids, the tip may thereby adopt a desired shape and, for example, the nature of the point may also experience a modification. This may take place by the resorbable material exposing a material lying underneath and adjacent, which has a different shape from the resorbable material. The material exposed in this way may likewise be resorbable, and for example also be more slowly resorbable, or non-resorbable.

Specifically, it is conceivable for the puncture needle tip to be formed by at least one resorbable material having a sharp point. If this material is then resorbed in the patient, for example upon blood contact or upon contact with other fluids, the puncture needle tip may thereby become blunt when a structure, which is configured for example without a sharp point and is cut in a straight line and consists of at least one non-resorbable material, is then exposed. It is thereby possible to create a puncture needle whose near-vein tip blunts itself, for example upon blood contact, and therefore can no longer inadvertently pierce the back wall of the vein, for example. It is also advantageous that the patient and the user can no longer be endangered when removing the now blunt puncture needle from the body and a further safety instrument, for example a piercing protector, is no longer needed.

It is likewise conceivable to use a material which experiences a modification, for example a shape change, due to a flow of gas, vapor or fluid. By way of example, the shape change due to such a flow may be caused by for example at least one molecular layer being ablated by the flow, and advantageously also transported away directly with the flow.

In this context, it is conceivable for there to be an element which intermittently or continuously over at least a particular period of time generates a flow of gas, vapor or fluid on the vein-remote end of the venous catheter and/or on a further component of the indwelling venous cannula, for example an extension element, which may for example be configured to be hollow. This flow may advantageously be directed toward the vein. An element which generates a reduced pressure and therefore a flow away from the vein, that is to say in the direction of the user, may however also be envisioned. The shape and/or point of the puncture needle may thereby be modified when pushing forward into the vein.

It is also conceivable that a structure exposed by resorption of another material is configured identically or similarly to a button cannula. In this case, the near-vein end of this cannula may, for example, be rounded or broadened.

It is also conceivable that the opening at the puncture needle tip and/or the lumen of the puncture needle is partially or fully kept open by at least one resorbable material. If this material is then resorbed, for example by blood contact or by contact with other fluids, the opening of the puncture needle tip or the lumen of the puncture needle is closed. This may, for example, be relevant when delivery of fluids or medications into a blood vessel is not desired but reaching a blood vessel with the puncture needle cannot be ruled out.

The effect just described may, for example, be achieved in that struts are formed by at least one resorbable material, which keep the opening at the puncture needle tip and/or the lumen of the puncture needle partially or fully open but no longer do so in the event of resorption.

It is also conceivable for the puncture needle to be reinforced on the inner and/or outer side of its wall with a resorbable material which keeps the wall in a position in which the opening at the puncture needle tip and/or the lumen of the puncture needle is partially or fully open.

In one advantageous version, at least a part of the puncture needle consists of resilient material.

It is, however, also conceivable that the opening of the puncture needle tip and/or the lumen of the puncture needle is narrowed in its diameter by at least one resorbable material and the opening and/or lumen experience(s) an increase of its/their inner diameter by the resorption of the material. This may be particularly relevant when detachment effects are intended to be avoided. Thus, by filling the lumen of the venous catheter with a resorbable material, it is possible to prevent constituents that are located in the insertion channel from being drawn into the vein or into more deeply lying tissue layers during the puncture. If such an effect is desired, it is advantageous for at least one resorbable material to predominantly narrow or completely close the lumen of the puncture needle in the original state. During resorption of the material by fluid contact, for example by contact with blood and/or contact with externally introduced fluids, an unperturbed flow of fluids may then by way of example take place through the puncture needle.

It may also be relevant for other applications, for example for applications in the spinal and epidural space. It is therefore proposed for puncture needles that are used in these applications advantageously to be developed in the manner just described. The resorbable material used may, for example, also be dissolved upon cerebrospinal fluid contact.

Particularly advantageously, a material which dissolves only upon cerebrospinal fluid contact but not upon blood contact may also be used. Inadvertent intravasal delivery/injection of fluids and also for example medications may thus be avoided, since the lumen of the puncture needle remains closed upon blood contact and therefore at a location in a blood vessel, for example in a vein. A catheter which, if appropriate, is intended to be inserted through the interior of the puncture needle cannot then accidentally be inserted into a blood vessel. An inadvertent catheter location in a blood vessel may therefore, for example, also be avoided.

The puncture needle may also consist of a grid-, network-, honeycomb-, labyrinth- or pore-like structure, which may contain at least one non-resorbable material and/or at least one resorbable material. Combinations of all structures mentioned may be envisioned.

The puncture needle may additionally be reinforced with further structures/elements, for example by transversely, longitudinally or diagonally running structures, which may contain at least one non-resorbable material and/or at least one resorbable material. Combinations of all structures mentioned may be envisioned.

At least one resorbable material may be introduced into the gaps of these structures. Such a structure may also fill the interior of a hollow puncture needle. A metal, including in the form of a metal alloy, or a plastic material or natural material may be used as non-resorbable material. For example, carbon fiber-reinforced laminate materials, polymers or Teflon may be envisioned as plastic materials, including in combination with one another. Aramids may also be used. The resorbable material may be a carbohydrate, or alternatively another biomolecule or a salt. This material may also be or contain magnesium. Resorbable polymers, composites, bioceramic materials or biodegradable metals may likewise be employed. The material may also be a combination of a plurality of resorbable materials, and also contain at least one antimicrobial or anticoagulant or otherwise effective or biologically active substance, or be coated with at least one such substance.

By blood contact or by contact with other fluids, the resorbable material may dissolve and structures which consist of at least one non-resorbable material may be left behind. The puncture needle and/or the interior of the puncture needle may thereby develop other properties, for example including filter-like properties. The fact that the dissolving of the resorbable material leads to changes of the flow properties creates a filter-like structure which may be adapted to the ambient conditions (for example variation of humidity) by changes of the permeability.

Conventional filter systems may also be integrated directly into the interior of the puncture needle. This likewise applies for tumefiable materials, for example for elastomers or for materials which to a certain extent or predominantly consist of fibers of vegetable origin and may have a different strength.

The puncture needle may advantageously be designed in lightweight construction. Lightweight construction materials may be used. In particular, metallic lightweight construction materials such as titanium, high-strength steel, aluminum and magnesium may also be used. Plastics and fiber-plastic compounds may also be used.

The puncture needle may consist partially, predominantly or fully of at least one material which is visible particularly well with the aid of ultrasound, or be coated with such a material. It is also conceivable that the puncture needle contains at least one channel in its wall, which is filled with a fluid, gas or vapor. A substance which is visible particularly well with ultrasound may be used for this, or alternatively for example air. This channel may be a channel running longitudinally over a partial length, the predominant length or the entire length of the puncture needle, which is configured to be impermeable to fluid, gas and vapor from its environment. The channel may, however, also be configured running transversely or diagonally and enclose the circumference of the puncture needle partially, predominantly or, in the sense of an annular structure, wholly. This structure may be arranged multiply on the puncture needle. In this case, the structures may be spaced apart from one another with equal or different spacings. The spacings between these structures may thus vary, in particular toward the puncture needle tip. In one advantageous version, the spacings decrease toward the puncture needle tip, that is to say the structures lie closer to one another. In particular, the location of the puncture needle tip may thereby be determined well with the aid of ultrasound.

All other sections of the puncture needle may partially, predominantly or fully have the properties just mentioned.

A puncture needle as described above may also be used independently of indwelling venous cannulas.

A venous catheter or another catheter or hose may also be configured as described above and have the properties described above.

### Further Aspects of the Resorbable Venous Catheter

Venous catheters and/or puncture needles may also consist over the entire length or a partial length of at least one material which is resorbable/dissolvable in a patient, for example by contact with blood or other fluids, gases or vapors, or be coated with at least one such material. The resorbable material may be a carbohydrate, or alternatively another biomolecule or a salt. This material may also be or contain magnesium. Resorbable polymers, composites, bioceramic materials or biodegradable metals may likewise be employed. The material may also be a combination of a plurality of resorbable materials, and also contain at least one antimicrobial or anticoagulant or otherwise effective or biologically active substance, or be coated with one such substance. It is conceivable for all components of the indwelling venous cannula to consist of a dissolvable material or be coated with such a material.

An initially present resorbable material may impart a certain hardness to the venous catheter and/or puncture needle, and the near-vein tip may also be configured to be hard by such a material so that it can be pushed forward well into the vein. Upon contact with blood, the material is resorbed, that is to say dissolved in the blood. Less hard or less pointed structures may thereby be released. Thus, a venous catheter may be configured to be hard before insertion into the patient, and in particular before insertion into the vein, and then become soft. A puncture needle may thus be configured to be sharp during insertion into the patient and the vein, and then become blunt.

Puncture Needle with Resorbable Outer Screw Thread

According to one advantageous configuration of the invention, it is provided that the puncture needle has an outer screw thread and/or the outer tunic has an inner screw thread. The outer screw thread and the inner screw thread may in this case be matched to one another in respect of size and pitch, so that the constituent part with the inner screw thread can be twisted in the manner of a screw in the constituent part with the outer screw thread.

It is likewise possible for the puncture needle to be developed with an outer screw thread consisting of at least one resorbable material. The venous catheter may then be provided with an inner screw thread fitting the latter, consisting of at least one resorbable material. It is also possible that only non-resorbable materials are used. The puncture needle and/or the venous catheter may then be twisted/screwed precisely into the vein.

The resorbable material may be a carbohydrate, or alternatively another biomolecule or a salt. This material may also be or contain magnesium. Resorbable polymers, composites, bioceramic materials or biodegradable metals may likewise be employed. The material may also be a combination of a plurality of resorbable materials, and also contain at least one antimicrobial or anticoagulant or otherwise effective or biologically active substance, or be coated with at least one such substance.

After twisting/screwing of the puncture needle on its own or together with the venous catheter into the vein, resorbable materials are resorbed by blood contact or by contact with other fluids. A smooth-walled puncture needle and a smooth-walled venous catheter are therefore again obtained.

A puncture needle as described above may also be used independently of indwelling venous cannulas.

### Resorbable Guide Wire

According to one advantageous configuration of the invention, it is provided that the puncture device has a guide wire which can be guided through the outer tunic or through the puncture needle, the guide wire being formed partially, predominantly or fully from at least one bioresorbable material.

A conventional guide wire may be introduced into the puncture needle or the venous catheter. It may be formed partially, predominantly or fully from at least one resorbable material. By way of example, the aforementioned materials may be used. The guide wire according to the invention is then at least partially resorbed and may therefore under certain circumstances be left in the body. Also, with a corresponding material selection, forgetting of the guide wire or shearing off of constituent parts thereof can no longer cause serious complications.

Twistability of the Puncture Needle about its Own Longitudinal Axis with a Spring Mechanism

According to one advantageous configuration of the invention, it is provided that the puncture needle is twistable in relation to the outer tunic about the longitudinal axis, the puncture device having holding elements by which the twistability of the puncture needle is restricted or negated at least in particular longitudinal displacement positions of the puncture needle in relation to the outer tunic.

According to one advantageous configuration of the invention, it is provided that the puncture device has at least one restoring spring, by which the puncture needle is held by a spring force in a longitudinal displacement position in which the twistability of the puncture needle is restricted or negated.

It is conceivable that a spring is located at the vein-remote end of the indwelling venous cannula, which in a relaxed (longer) state holds the puncture needle in a longitudinal position such that the puncture needle is impeded from twisting about the longitudinal axis by holding or path limiting elements which are fitted on the puncture needle itself and on at least one further component of the indwelling venous cannula. The holding or path limiting elements may in this case be configured for example as notches, furrows or projections. If the puncture needle is then actively displaced with pressure against at least one other component of the indwelling venous cannula in the longitudinal direction in the direction of the vein, the spring is compressed. The holding or path limiting elements of the puncture needle and the at least one further component of the indwelling venous cannula then no longer block the twisting movement of the puncture needle about the longitudinal axis, since for example they are no longer latched in one another or wedged in one another. The puncture needle can then be twisted about its own axis in the longitudinal direction. If the pressure on the puncture needle is then released, the spring relaxes and the puncture needle is displaced passively in the direction of the user (away from the vein). The holding or path limiting elements then again impede a twisting movement of the puncture needle about its own longitudinal axis in the manner described. For example, a kind of “childproofing” may thereby be provided.

The venous catheter of the indwelling venous cannula may likewise be twistable as just described. This may have the advantage that, for example, components of the venous catheter which are obstructed/blocked by anatomical structures, for example entry or exit holes, can be twisted/moved away from the obstructing/blocking structures without the entire indwelling venous cannula having to be moved or removed.

The venous catheter may for this purpose be mounted twistably about its own longitudinal axis in or on the indwelling venous cannula. The venous catheter may likewise transition into a structure with a larger inner or outer diameter at its vein-remote portion.

This may likewise be mounted twistably about its own longitudinal axis in relation to at least one component of the indwelling venous cannula. There may be a longitudinal displaceability of this structure. It is also conceivable that a further structure independent from the venous catheter, which has a larger inner and outer diameter, is located in the region in which the venous catheter transitions remotely from the vein into the body of the indwelling venous cannula. This corresponds to the region of the fastening element 5 and of the holding element 8. This structure may reduce or wholly impede the twistability of the venous catheter about the longitudinal axis.

By providing a clamping element, the individual layers of the puncture system may be partially or completely fixed in relation to one another in order to be able to ensure a secure location of the puncture system. The clamping element may in this case be configured as a kind of pincer having a conically slotted tunic and a union nut, the individual layers being pressed into the inner cone of the tunic and therefore tensioned by pulling the union nut. The following alternatives may, however, also be envisioned:
An expansion body, which is introduced into the puncture system in order to fix the individual layers, the inner-lying layers being pressed against the outer-lying layers and therefore fixed by expansion of the expansion body.
A clip system, the individual layers being fixed by pressure in relation to one another by folding down a clip located outside the puncture system.
A screw, which is introduced into the puncture system through a corresponding bore, the screw fixing the various plies by pressure in relation to one another by twisting.
A coating of the individual layers of the puncture system may be adapted so that the longitudinal displaceability is restricted in comparison with the conventional system.
It is also possible that the individual layers are latchable into one another, for example by notches.
The individual layers may be wedgeable in one another by their having a non-circular diameter or a diameter increased at individual sites, and at least partial fixing of the individual layers in relation to one another therefore takes place by twisting or pushing forward and pulling back.
The clamping element may be configured in such a way that all layers are fixed in relation to one another in an untouched position, for example by a spring mechanism. By pressure or traction on the clamping element, the fixing of the layers is negated and the spring is tensioned. If the clamping element experiences no pressure or traction, it returns because of the spring mechanism into its initial position and fixes all the layers against one another. This embodiment of the clamping element has the advantage that, for example, the clamping element can be operated with only one finger.

The described functions of the fixing element are controllable and repeatable, so that great flexibility of the fixing element is guaranteed. In particular, the fixing element is also intended to ensure that the puncture system can be adapted to particular living beings and their individual anatomy, or to the anatomy of different body regions. The presumed subcutaneous depth of the structure which is intended to be punctured may already be taken into account before the start of the puncture process, in that the sharp portion of the puncture needle protruding from the inner vasiform body can be limited in its free length to the living being to be punctured. The risk that anatomically deeper-lying structures will accidentally be punctured, in particular by an inexperienced user, is thereby also reduced. This represents a substantial safety aspect of the new puncture system.

The puncture needle and the venous catheter may also be connected to one another so that they can be twisted only together/in combination. It is conceivable for at least one friction-increasing material to be located on the outer side of the puncture needle and/or on the inner side of the venous catheter in order to achieve this effect.

The puncture needle and/or venous catheter may preferentially be capable of being set to a circular measurement of 45°, 90°, 135°, 180°, 225°, 270°, 315° or 360° (angle specifications in degrees with respect to a circular measurement of 360 degrees (360°)).

The angle specifications may be made discernible for the user on a component of the indwelling venous cannula, so that for example the current angle of the puncture needle or the venous catheter can be read off. Here, for example, circular or semicircular markings in a different line width and, for example, also, markings in the traffic light colors “green”, “yellow” and “red” may be envisioned.

Markers Detectable with an Imaging Examination Apparatus

According to one advantageous configuration of the invention, it is provided that at least one constituent part of the puncture device, in particular the outer tunic, has at least one marker that can be detected by an imaging examination apparatus. There may also be a plurality of markers, both on the same constituent part or on different constituent parts of the puncture device. The markers may be identical or different markers, which are detected by different types of imaging examination apparatuses. For example, there may be at least one marker which can be detected by an ultrasound examination apparatus and/or at least one marker which can be detected by computed tomography, and/or at least one marker which can be detected by magnetic resonance tomography. In one advantageous configuration, the at least one marker is located in a region of the puncture device that is adapted for insertion into the hollow body to be punctured, i.e. in the region near to the hollow body.

According to one advantageous configuration of the invention, it is provided that at least one constituent part of the puncture device, in particular the outer tunic, has at least one cavity which is filled or can be filled with air or another substance that can be detected with an imaging examination device. Such a cavity may, for example, function as at least one marker in the sense described above. The at least one cavity may be fully closed hermetically from the external surroundings or have at least one opening to the surroundings, for example to the outer side of the outer tunic. In one advantageous configuration, the at least one opening is located in a region of the puncture device which is not adapted for insertion into the hollow body to be punctured, i.e. in the region remote from the hollow body.

### Supplement Relating to Piercing Resistance

It is proposed that at least the part of the venous catheter which is adapted to remain in the patient is formed from a piercing-resistant material or coated with such a piercing-resistant material over a partial length, its entire length or the predominant part of its length in which the puncture needle can be guided longitudinally displaceably.

### Supplementary Aspect Relating to the Piercing-Resistant Hollow Seldinger Wire

By a significantly elongated venous catheter, which is configured in the sense of a hollow piercing-resistant Seldinger wire, a puncture needle may in principle be brought to any place of the body without there being an injury risk for the anatomical structures through which it passes on the way to the target structure. Such a significantly elongated venous catheter may also be used independently of an indwelling venous cannula. It may for example have a length of at least 200 mm, 300 mm, 400 mm, 500 mm, 600 mm, 700 mm, 800 mm, 900 mm, 1000 mm or even up to 2000 mm. All lengths between these measurements may be envisioned.

If this venous catheter or hollow wire is configured to be fluid-impermeable, for example with a corresponding coating or cladding, which may for example consist of PTFE, it may also be used as an aspiration element. With this aspiration element, by way of example, blood clots or other foreign bodies may be aspirated from any anatomical structures that can be reached with the aspiration element and may be removed from the anatomical structures. By way of example, blood clots may be extracted from blood vessels near to the brain, heart and extremities. A further aspiration element, for example a syringe, may be attached at the vein-remote portion of the hollow Seldinger wire formed in this way, for example by means of a correspondingly configured connector. Attachment to a precisely controllable aspiration element may also be envisioned.

One aspect of the invention therefore also relates to the version of the puncture device in the form of a hollow spiral wire having a fluid-impermeable coating, which is configured to be extremely long, for example at least 1000 mm. This hollow spiral wire may have an attachment element for attaching a suction device at the end remote from the patient, for example a syringe, a motor-driven suction device or a similar element. For example, a coronary suction device may thereby be provided. This relatively long hollow spiral wire may by way of example be provided in a state wound to form a roll and/or with a cladding consisting of a sterile tunic.

### Self-Closability of the Venous Catheter

According to one advantageous configuration of the invention, it is provided that the outer tunic has a fluid-permeable lattice-, network- or pore-like structure with fine openings, which can be closed by blood constituents flowing through during operation of the puncture device. Such an outer tunic having fine openings initially, for example in the production state, allows fluids, in particular blood, to flow through. By particular blood constituents which are incorporated into the gaps of the structure, closure of the openings then automatically takes place so that the outer tunic then becomes fluid-impermeable.

This may also entail an increased resistance against pressure.

In the original state, the venous catheter may also consist of a fluid-permeable lattice-, network- or pore-like structure, which may also be developed by further structures, for example horizontally, diagonally or vertically running structures. After the insertion of the catheter, blood constituents may then be incorporated into the gaps of the structures so that the venous catheter is configured to be fluid-impermeable. This creates a venous catheter which forms biological properties and may be left for a longer time or permanently in the vein.

### Electrically Insulating Elements on the Indwelling Venous Cannula

According to one advantageous configuration of the invention, it is provided that at least parts of the puncture device have electrically insulating elements and/or electrically conductive elements, by which electrically conductive connections from one part of the puncture device to another part of the puncture device are formed.

It is also conceivable that the indwelling venous cannula contains insulating, for example electrically insulating, elements or materials, which may for example prevent development of heat that occurs because of external influences on the indwelling venous cannula. These elements or materials may, for example, enclose a sensor which may be fitted on the venous catheter. These elements or materials may, for example, also enclose a spiral or undulating structure and be introduced therewith into the venous catheter or form the venous catheter therewith.

### Electrically Conductive Elements on the Indwelling Venous Cannula

The entire indwelling venous cannula may be formed partially or fully from at least one electrically conductive material or be coated with such a material. It is also conceivable that a longitudinally running structure which is electrically conductive, configured for example in the form of a strip, may run on or in the venous catheter.

The electrically conductive material may, for example, be a metal or a metal alloy. The following materials may be used by way of example: gold, silver, copper, brass, tungsten, aluminum, lead, stainless steel, iron, zinc, chromium, stainless steel, beryllium, platinum, nickel, titanium. Carbon/graphite may also be employed.

### Other Embodiments of the Near-Vein End of the Venous Catheter

According to one advantageous configuration of the invention, it is provided that the outer tunic has a greater outer diameter in the region near to the hollow body than in the region remote from the hollow body.

The venous catheter may be enclosed over a partial length or its predominant or entire length by a stamp-shaped/cylindrical structure, in relation to which it may also be longitudinally displaceable. This structure may experience a significant increase in its outer diameter in the direction of the vein and therefore stabilize the puncture needle and the venous catheter during insertion through the skin and into the vein.

During insertion of the puncture needle and of the venous catheter, this structure may be displaced on the venous catheter in the direction of the user, since it cannot pass through the skin. It may likewise have telescopic properties. The puncture needle and the venous catheter may likewise be mounted displaceably, tiltably and/or twistably in relation to this structure in various directions.

The venous catheter may be enclosed semicircularly or circularly in the stamp-shaped/cylindrical structure by a further structure, in relation to which it may be mounted longitudinally displaceably and/or twistably. It may likewise be enclosed by a bag-shaped structure. This structure enclosing the venous catheter may keep the venous catheter sterile and, for example, also contain an antimicrobial, anticoagulant and/or friction-reducing substance.

### Volume Increase of the Venous Catheter

According to one advantageous configuration of the invention, it is provided that at least one region of the outer tunic has a material which swells by contact with a fluid. Such a swelling material may, for example, be a tumefying material, for example a hydrogel. When a fluid is mentioned in this application, this includes both liquid media and media in the form of a gas or vapor.

The venous catheter may likewise consist partially of at least one material which experiences a volume increase by blood contact, or very generally by contact with fluids, gases or vapors, or by at least one chemical reaction, or be coated with such a material. By way of example, polymers, sheet silicates, bentonite or compound materials which contain natural fibers may be used. Proteins, fats or superabsorbers may likewise be used.

It is likewise possible for the venous catheter to consist over a partial length or its predominant or entire length at least partially of a material which experiences a volume increase by blood contact, or very generally by contact with fluids, or be coated with such a material.

If this material is located on the outer side of the venous catheter, the venous catheter itself fixes its position in the vein by an increase of its outer diameter, in particular when a further solid material on the inner side of the venous catheter allows only an outward volume increase of the venous catheter. If, however, the volume-increasing material is present on the inner side of the venous catheter and a solid material which prevents the outward volume increase is present externally, the inner diameter of the venous catheter is reduced and it thus closes itself.

### Increase of the Inner Diameter of the Venous Catheter by Blood Contact

Parts or layers of the catheter may also consist of a material which is resorbable, for example blood-soluble. This material may be a carbohydrate, or alternatively another biomolecule or a salt. This material may also be or contain magnesium. This soluble material may form any desired layer of the venous catheter. If it forms the inner layer of the venous catheter, for example, the inner diameter of the venous catheter increases by blood contact.

### Restricted Longitudinal Displaceability of the Puncture Needle

According to one advantageous configuration of the invention, it is provided that the puncture device has an automatic blocking mechanism, by which the puncture needle is prevented from being pushed forward toward the end near to the hollow body when the puncture needle has been withdrawn beyond a predetermined extent from the end near to the hollow body.

In order to prevent the puncture needle from being pushed forward into the vein again after the puncture needle has already been withdrawn from the vein, a mechanism that prevents this may be formed on the puncture needle. This mechanism may be located at any desired site of the puncture needle, but in an advantageous development at a site which is located on the (vein-remote) half of the puncture needle turned toward the user, in relation to the longitudinal extent of the puncture needle. In a further advantageous development, this site is formed in the near-user last third, last fourth or last fifth of the puncture needle.

Such a mechanism is important since pushing the puncture needle forward again in the direction of the vein may also lead to shearing of constituent parts of the venous catheter by the tip of the puncture needle.

The mechanism may be formed by at least one ramp-shaped structure being located on the puncture needle. This structure may be located on the surface of the puncture needle or introduced in a recess of the puncture needle. The ramp-shaped structure may contain and/or be connected to at least one spring-shaped and/or spiral structure.

If the puncture needle is located in its original maximally near-vein position before and during the puncture process, the structure just described is enclosed by the venous catheter or further hollow elements/components of the indwelling venous cannula which extend the venous catheter in the direction of the user in the longitudinal direction. The structure cannot therefore be raised above the level of the surface of the puncture needle. The spring connected to the structure remains compressed.

If the structure leaves the venous catheter or the further hollow elements/components of the indwelling venous cannula, however, relaxation of the spring causes the structure to be raised above the level of the puncture needle. This takes place to such an extent that the puncture needle can then no longer readily be pushed forward in the direction of the vein since the structure no longer fits into the venous catheter or the further hollow elements/components of the indwelling venous cannula.

Instead of the ramp-shaped structure, a button- or stamp-shaped structure may also be used.

It is also conceivable to use a bent structure without a spring mechanism. This may also have properties of a bimetal and, for example, be raised from the surface of the puncture needle by the body heat by increasing its curvature.

### General Material Properties

The indwelling venous cannula may advantageously be developed by way of example by the following substances or substance classes, constituents, materials, elements etc. (referred to below as “materials”) individually or in combination. The materials mentioned are in this case not categorized systematically and describe both material and functional properties. Overlaps, in particular between a material and functional description, as well as in the actual terminologies, are possible. It is likewise explicitly possible for all components of the indwelling venous cannula to be formed from the following materials, or contain them, individually or in combination with one another:
biocompatible metals and polymers, biopolymers
biocompatible plastics, explicitly for medical use
shape-memory alloys, for example nitinol (nickel-titanium alloy)
copper, zinc, aluminum, iron, tungsten, manganese, silicon, magnesium, cobalt, gold, silver, bronze, platinum, palladium
brass
stainless steel, steel alloy, in particular rust-free
unfired bricks
functional polymers or intelligent polymers, for example shape-memory polymers and thermoresponsive polymers
ceramic materials, including bioceramics, textile fiber ceramics
silicone or silicone elastomers
chromium
thermoplastics
thermosets
elastomers, thermoplastic elastomers (TPE), TPE-A
polyimides
polyolefins
resins and synthetic resins
rubber, including special rubber, including EDPM
natural rubber
latex
aramids, aramid fibers, para- and meta-aramid fibers
paperboard
natural fibers
mineral fibers
fiber composites, fiber- and textile-reinforced compounds
carbon fibers, which may also reinforce composites
technical textiles
urethanes, polyurethanes, including thermoplastic polyurethanes (TPU)
polyester (PES)
polyvinyl chloride (PVC)- and latex-free materials
sand, for example quartz sand
wood
basalt
fluoropolymers
polyurethane elastomers
high-performance thermoplastics
polystyrene (PS), including expanded PS
polyamide (PA)
polycarbonate (PC)
perfluoroalkoxy polymers (PFA)
polysulfone (PSU)
polyether block amide (PEBA)
polyether ether ketone (PEEK)
polyoxymethylene (POM)
polyphenylene sulfone (PPSU)
polypropylene (PP)
polyethylene (PE), including LDPE, HDPE
polyether imide (PEI)
polyethylene terephthalate (PET), Dacron
polyvinylidene fluoride, polyvinylidene difluoride (PVDF)
fluorinated ethylene propylene (FEP)
polyphenylene sulfide (PPS)
polyphthalamide (PPA)
acrylonitrile butadiene styrene (ABS)
methyl methacrylate-acrylonitrile butadiene styrene (MABS)
ethylene vinyl acetate (EVA), EVAC
elastane
melamine formaldehyde resin (MF)
polyester resin
phosphorylcholine
nylon
Teflon
bentonite
glass, fiberglass, glass fiber-reinforced plastic
UV adhesive
PTFE, expanded PTFE, porous PTFE, Polytetraflon® PTFE
ETFE
parylenes
aminoplasts
carbohydrates, sugars
proteins
fats
carbon
wool
Mylar®, Kapton®, Nomex®, Kevlar®
nonwoven, nonwoven fabric
chitin

The venous catheter may contain at least one spiral and/or undulating structure which consists of at least one of the materials just mentioned and/or is coated with at least one such material and/or is enclosed partially, predominantly or fully by at least one such material.

The use of compound materials and layer compounds is explicitly possible, and hemocompatibility and an anti-adhesive behavior is advantageous for all materials used. Likewise advantageous are sterilization resistance and security against fracture. Advantageously, materials with thermoplastic properties may be used, as well as those which are visible in X-raying. A UV stability and a high resistance against chemical influences are to be sought.

The materials may advantageously be combined so that the entire indwelling venous cannula or the venous catheter is configured to be resistant against piercing, kinking and cutting, as previously described in PCT/EP2019/057097. Fabric inlays may also be used.

Likewise possible is the use of fibers and biologically degradable materials. Films and laminates may also be used.

Likewise, resorbable/dissolvable materials or materials that release medications or substances may be used. For example, it is possible that materials release antimicrobial, antiinflammatory, chemotherapeutically or locally anesthetically effective substances, contain them or are coated therewith. This also applies for example for antithrombogenically effective substances, for example heparin, which can hinder the formation of blood clots on or in the vicinity of the venous catheter.

The surface structure and chemistry of the surface may be optimized. A hydrophilic or hydrophobic coating may be advantageous. A superhydrophobic or ultrahydrophobic surface may also advantageously be employed. The plasma coating process can be used for production. It is also conceivable for the surface of components of the indwelling venous cannula, for example the surface of the puncture needle or of the venous catheter, to be partially, predominantly or fully provided with polymer brushes.

The aforementioned materials may also be employed in a locally varying fashion on the venous catheter. For example, it is conceivable for a different material to be used at the immediate near-vein end of the venous catheter than at the vein-remote region. By way of example, the tip of the venous catheter may be formed from a softer material having thermoplastic properties. The tip of the venous catheter may, however, also be configured deliberately from a harder material in order to facilitate the pushing forward into the vein.

At one or more sites of the indwelling venous cannula, materials may also be connected to one another or merge into one another by technical processes. For example, it is possible to connect or fuse a spiral spring at the immediate near-vein end of the venous catheter to an inner- or outer-lying material enclosing it. Such connection or fusion may take place in the region of the entire venous catheter. Breaking of the spiral spring may thereby be prevented. Advantageously, materials having a suitable toughness may be used in this case.

Advantageously, materials which expand at body temperature or tumefy in the environment of a fluid, that is to say increase in volume, may also be employed. For example, bentonite may be used. Such an effect may for example be used at the near-vein end of the venous catheter: when the venous catheter is introduced, it is relatively sharp-edged at the near-vein end and bears tightly on the puncture needle. When the venous catheter is installed as intended in the vein, it then tumefies at the near-vein end and can thus damage the vein wall less. It is also possible that the venous catheter can tumefy over a further part or over the entire length. It is also possible that the puncture needle can tumefy as just described and therefore changes its properties, for example blunts itself and is no longer sharp at the near-vein end. Hydrogels may be used.

It is also possible to apply further structures, for example coil-like structures.

The use of a plurality of layers of different materials, which for example form the wall of the venous catheter, is also explicitly possible. These layers may be firmly connected to one another or displaceable in relation to one another. Different aggregation states of materials may be present in the wall of the venous catheter.

When suitable materials are used, the indwelling venous cannula or components thereof may be adapted to the individual profile or the individual anatomical configuration of the vein, for example by bending, before insertion into a patient by the user.

Refinement of the layers mentioned in the last section or of the surfaces is explicitly possible.

Materials or surface coatings may have fluorescent properties. Materials or surface coatings may also be stained or very generally modified by metabolic activities, for example by metabolic activities of germs, for example bacteria. Staining or modification by the patient's own metabolic activities may, however, also be envisioned. For example, the installation time of an indwelling venous cannula or of a venous catheter may thus be established and monitored.

The intactness may also be verified in this way. It is conceivable that the material properties of the venous catheter change during a prolonged installation time or in the event of material defects, for example breaks or cracks in the venous catheter. For example, the electrical conductivity may thus vary. If a current is then applied via a conductive venous catheter, it is possible to establish whether the venous catheter has damage. It is also conceivable for the venous catheter to change properties that make it less, more or differently visible in an ultrasound or X-ray image. In the present text, this fundamentally also always includes representation in other imaging methods, for example computed or magnetic resonance tomography.

Thus, particularly in the case of venous catheters/indwelling venous cannulas installed in the patient for a prolonged period of time, regular material inspection may be carried out. The installation time may thus also be monitored. It may also be particularly relevant for the venous catheter to have properties that make it less, more or differently visible in an X-ray or ultrasound image in the event of germiness. For example, it is thus possible to establish whether a venous catheter might be a source of blood toxicity (sepsis). Thus, unnecessary catheter changes may potentially also be avoided.

Electrically charged materials may also advantageously be used. For instance, it is conceivable that the venous catheter is electrically charged, for example positively charged, by special material properties of its outer side and is therefore automatically repelled or moved away from the more negatively charged vein inner wall (intima). Potential damage to the vein wall may then be avoided by reduced mechanical irritation. The venous catheter may also be externally provided with a small current or a small voltage, so that this effect is further enhanced or made possible in the first place in a sufficient intensity.

Nanoparticles may likewise be used.

The aforementioned materials may also all be employed in all sections, parts and components of the indwelling venous cannula which are adapted to remain outside the patient.

The entire indwelling venous cannula or components thereof may be designed in lightweight construction.

### Specific Use as a Pleural Catheter

According to one advantageous configuration of the invention, it is provided that the puncture device is configured as a pleural catheter. It is in this case advantageous for the puncture device or at least the outer tunic to be configured to be piercing-resistant and/or kink-resistant. The piercing resistance may, for example, be achieved by a corresponding piercing-resistant coating or by configuration as a spiral hollow wire. The spiral hollow wire may in this case be wound very tightly. In one advantageous configuration, the turns of the spiral hollow wire bear directly on one another without a spacing. In another advantageous configuration, the turns are spaced apart from one another by at most 0.1 millimeter, at most 0.5 millimeter, at most 1 millimeter or at most 2 millimeters, in which case the spacing between the turns may vary. In order to make the spiral hollow wire furthermore piercing-resistant, at least one further piercing-resistant material may be located between the turns. The piercing resistance of the material in this case refers to possible piercing of the pleural catheter by the puncture needle, which is intended to be prevented by the piercing-resistant material. The piercing-resistant material may be a metal, including in the form of a metal alloy, or a correspondingly piercing-resistant plastic material or natural material. For example, carbon fiber-reinforced laminate materials, polymers and/or Teflon may be envisioned as plastic materials, including in combination with one another. Aramids may likewise be employed.

The piercing resistance ensures that parts of the puncture device cannot be pierced or sheared off by passing a conventional puncture needle through. The kink resistance ensures that accidental kinking of the puncture device does not occur during intended use.

In the configuration as a pleural catheter, the catheter system is designed so that it can be used to puncture the pleural gap, for example in the case of morbid accumulations of gases, vapors or fluids in the pleural gap (for example pneumothorax, hemothorax, pleural effusion). These may then be evacuated from the pleural gap with the aid of the catheter system. In this application as well, a puncture needle (in this application a pleural puncture needle) may be guided through a catheter (in this application pleural catheter) enclosing it. The catheter may in this case be configured as described in this patent application and in PCT/EP2019/057097.

It is advantageous for the pleural catheter to have a piercing length of at least 50 mm, for example 80 mm, 85 mm, 90 mm and at most 140 mm. The pleural catheter may, however, also have piercing lengths of up to 160, 180, 200 or 600 mm. Pleural puncture needles and pleural catheters and the entire catheter system as a whole are in this case significantly elongated. The piercing length of the pleural catheter is in this case the length which corresponds to the length of the pleural catheter lying in the patient. For application in special patient groups, for example children, this may also be less than 50 mm. All lengths may be envisioned. The outer diameter of the pleural catheter may be 2-5 millimeters, in one advantageous version 3-4 millimeters. The inner diameter of the pleural catheter may be 1-4 millimeters, in one advantageous version 1.5-3 millimeters. All sizes may be envisioned.

All versions/properties just mentioned may also be employed in and on a general puncture system, for example including an indwelling venous cannula.

### Further Applications

In one advantageous version/development, the indwelling venous cannula is a catheter system for invasive measurement of the arterial blood pressure.

In one advantageous version/development, the indwelling venous cannula is a universal blood vessel catheter system that may be used for all blood vessels. In this case, the catheter system may be provided with a marking system that alternatively adjustably comprises the elements/colors “blue” and “red”. “Blue” in this case stands for a venous location, and “red” for an arterial location of the catheter lying in the corresponding vessel.

In one advantageous version/development, the indwelling venous cannula is a catheter system for puncturing the trachea.

In one advantageous version/development, the indwelling venous cannula is a catheter system for puncturing the urinary bladder.

In one advantageous version/development, the indwelling venous cannula is a catheter system for puncturing the gastrointestinal tract and/or the abdominal cavity.

In one advantageous version/development, the indwelling venous cannula is a catheter system for puncturing reservoirs, pump, hose, tube and port systems.

In one advantageous version/development, the indwelling venous cannula is a catheter system for use in the field of interventional radiology.

In one advantageous version/development, the indwelling venous cannula is a catheter system for use in the field of interventional cardiology.

In one advantageous version/development, the indwelling venous cannula is a catheter system for use in the field of emergency, disaster, tactical and military medicine.

### Thumbpiece

The indwelling venous cannula may have at least one holding and/or attachment element (in what follows, only “holding element”) to facilitate application on a patient, the holding element having a complementary shape or a negative contour of a human thumb or a human fingertip. The indwelling venous cannula may thereby advantageously be developed ergonomically for the user.

The holding element may, in particular, also be configured to be bent/curved. For example, it may be configured convexly in the direction of the vein and concavely in the direction of the user. It is, however, also possible for it to be configured concavely in the direction of the vein and convexly in the direction of the user. It is also conceivable that only the side turned toward the user is configured to be bent/curved and that the side turned away from the user is configured to be straight. Likewise, the side turned toward the user may be provided with at least one notch, flattening and/or furrow in order to receive a finger, the fingertip and/or the fingernail of the user.

In such a case, one-handed operability of the indwelling venous cannula may be facilitated since one finger, for example the index finger, of the user can reliably push the attachment element and the venous catheter connected thereto in the direction of the vein. The holding element may, but need not, be configured so that an aspiration element, for example a syringe, can be attached. For example, it may also be configured cylindrically, in the form of a stamp or in the form of a plate, or in the form of a polygon and only the side turned toward the user as described above.

The holding element may also be fitted on the other components of the indwelling venous cannula in such a way that it is partially or fully twistable.

The attachment element may also be fitted on the other components of the indwelling venous cannula so that it is partially or fully tiltable toward one, two or more sides.

The attachment element may also be fitted on the other components of the indwelling venous cannula so that it is partially or fully displaceable in the longitudinal direction.

The attachment element may consist on its upper side of at least one material which is distinguished by an increased frictional resistance, or be coated with such a material. By way of example, the surface structure may have ribs or pimples. It may likewise have adherent or adhesive properties and/or be provided with a peelable protective film.

There may likewise be a plastic deformability of the holding element. The deformable material may also be partially or fully detachable from the other components of the indwelling venous cannula.

The attachment element may likewise contain a coupling element. By means of the latter, it may be detachably connected to the other components of the indwelling venous cannula. This coupling element may also be configured in the form of a plug connection. The attachment element may likewise be detachably or inseparably connected to a 2nd attachment element. It is also conceivable that the attachment element is also configured in the manner just mentioned only partially, particularly in the direction of the user.

The aforementioned developments have the advantage that the indwelling venous cannula no longer needs to be grasped in the vicinity of the venous catheter, and the risk of contamination with germs is thereby reduced.

It is also conceivable that the attachment element has a flatly running inner channel which meets the attachment to the venous catheter at a shallow angle. The height of the attachment element may thereby be reduced. It is then possible to set an aspiration element at an acute angle, with respect to the longitudinal axis of the venous catheter, by means of the attachment element onto the indwelling venous cannula. An infusion line, for example, may likewise be fastened at the acute angle as just described on the indwelling venous cannula. A further hollow extension element, through which a fluid can flow, may then be obviated. The acute angle has, for example, the advantage that no holding and/or aspiration element projects steeply upward from the indwelling venous cannula. The acute angle also has the further advantage that, for example, an aspiration element and/or an infusion line does not need to be connected on the indwelling venous cannula directly in the vicinity of the skin, which may cause contamination of the connection site with germs.

The attachment element may have an inner and/or outer screw thread and, for example, be compatible with the attachment elements of aspiration elements and/or infusion lines, which may likewise contain screw-threaded structures.

### Removable Layers of the Venous Catheter/Hollow Structure

According to one advantageous configuration of the invention, it is provided that the outer tunic is configured in multiple layers with one or more removable layers.

The venous catheter can also be configured with one or more removable layers. If these are removed, the inner diameter or the lumen of the venous catheter is increased. In this way, layers of the venous catheter which are colonized with germs or have blood clots may be removed easily. It is, however, also possible that layers may be introduced from the outside in this way and the venous catheter may therefore be supplemented with further layers. It is also possible to use a hollow structure which can be introduced into the venous catheter and can be regularly changed. This may be adapted or adapt to the respective inner diameter and the geometrical shape of the venous catheter. The hollow structure may, however, also form a geometrical structure which is different to that of the venous catheter. The hollow structure may consist of at least one layer. This at least one layer may have antithrombogenic, antimicrobial and/or piercing-resistant properties. The hollow structure may also have a spiral or undulating surface and be developed with a sealing coating, for example a PTFE coating.

### Further Versions of the Venous Catheter

It is also conceivable that the venous catheter contains structures resembling fish scales or roof tile-like structures, which may for example be arranged overlapping or overlaid.

The venous catheter may in this case also be formed in a similar way to a commercially available shower hose, likewise as a braided hose. The venous catheter may advantageously be developed by various types of braid. For example, various spiral braids, which may for example consist of stainless steel or another metal or a metal alloy, may be envisioned here. Flat braids, core-cladding braids or packing braids are also conceivable, if appropriate in modified forms. The braids may, however, also consist of any other material, in particular a piercing-resistant and cutting-resistant material, or be coated with such a material. It is also conceivable to form the venous catheter with a metal fabric, an expanded sheet or expanded metal.

The venous catheter may also have a honeycomb-, grid- or pore-like structure. It may likewise be configured with multidimensional fabric structures.

The venous catheter may also additionally be reinforced with longitudinally running structures. These may be arranged over a partial length, the predominant length or the entire length of the venous catheter or only in the region of the near-vein portion of the venous catheter. These longitudinally running structures may, for example, promote pushing of the venous catheter forward into the vein during the insertion process and stabilize the inner-lying rather flexible venous catheter, for example so that it is not pushed against the vein wall by the blood flow.

The venous catheter may also be configured as a shrink hose or clad with such a hose. The latter may be a PTFE hose. The venous catheter may also be configured as a multilayer hose.

The venous catheter may be a rubber hose or a rubber tube.

### Three-Way Stopcock/Filter

The indwelling venous cannula may already contain at least one integrated three-way stopcock or a multi-way stopcock system, which may respectively be connected detachably or inseparably to the indwelling venous cannula. In particular, it may be connected inseparably to a hollow extension element following on from the venous catheter in the direction of the user. The three-way stopcock may also be configured as a three-way stopcock with a hose. At least one filter element may be connected detachably or inseparably to at least one component of the indwelling venous cannula, and in particular to the hollow extension element.

### Further Versions of the Spiral/Undulating Structure

According to one advantageous configuration of the invention, it is provided that the wall of the outer tunic has at least two spiral or undulating structures, in particular one spiral or undulating structure directed toward the inner side of the outer tunic and one spiral or undulating structure directed toward the outer side of the outer tunic.

The wall of the venous catheter may also be formed by one at least double spiral or undulating structure, or at least two spiral or undulating structures may be located in the wall of the venous catheter.

These may be connected inseparably to one another, or alternatively by design they may have no connection to one another. In the latter case, they may be movable in relation to one another, for example displaceable in the longitudinal direction, or twistable/rotatable in relation to one another. This has the advantage that the venous catheter keeps its flexibility and can therefore be adapted to the vein profile, but it is nevertheless configured more robustly/more stiffly.

It is also conceivable that, in particular, a second spiral or undulating structure is introduced into the indentations of a first and/or engages therein. The structures may in this case be movable in relation to one another, although they may also be restricted in their mobility in relation to one another by at least one material and/or coating which increases the frictional resistance. A restriction of the mobility in relation to one another may also be carried out such that there are further connections or structures, for example microstructures or nanostructures, between the two spiral or undulating structures.

A combination of at least one spiral or undulating structure and at least one braid-like structure may also be envisioned. An undulating surface is embodied at least in cross section along the longitudinal axis of the venous catheter by alternating diameters of the venous catheter. A waveform may in this case consist, for example, of a sinusoidal, rectangular, triangular and/or sawtooth oscillation.

The outer diameter of at least one spiral structure may vary over the length of the venous catheter. In particular, the outer diameter may decrease in the direction of the vein. It is, however, also conceivable for the outer diameter to be greater in regions of the venous catheter which are particularly loaded mechanically, for example where the venous catheter is connected to further components of the indwelling venous cannula and/or is guided through the skin of the patient after installation, than in other regions. Here, corresponding formation of the spiral structure over a length of 1-20 millimeters, and in particular 2-10 millimeters, may be advantageous. The outer diameter may in this case be increased at least 1.5 times, or alternatively at least doubled.

In combination with a variation of the tightness of the turns, a venous catheter may thus be provided which is flexible but at the same time also robust and adapted to different situations, for example anatomical situations.

The spiral or undulating structure may consist over its partial length, predominant length or entire length of nitinol (nickel-titanium alloy), stainless steel, aramids and/or at least one hard plastic material, or have a corresponding coating.

The spiral or undulating structure may consist over its partial length, predominant length or entire length of magnesium materials or have a corresponding coating that contains magnesium. In this way, resorbable properties may be imparted to the structure. In particular, longitudinally, transversely or diagonally running structures which stabilize the venous catheter during its insertion into the vein may thereby also contain magnesium. These may then be resorbed, for example by the blood flow, after the insertion of the venous catheter into the patient.

This creates a venous catheter which is configured stiffly during insertion into the vein and then becomes increasingly softer. As an alternative or in addition, the venous catheter may be coated on the inner and/or outer side with a corresponding material in addition to, but also independently of, the use of a spiral or undulating structure. If such a structure is used, however, it may also be fully enclosed by at least one material that contains magnesium.

It is, however, also conceivable that all other components of the venous catheter and of the indwelling venous cannula consist at least partially of magnesium materials or have a corresponding coating that contains magnesium.

A spiral structure may consist of at least one material that expands at body temperature in comparison with room temperature. A tighter winding and therefore a variation of the flexibility of the spiral structure when warmed to body temperature, for example when installing the venous catheter in the vein, may thereby be achieved. A venous catheter which contains the spiral structure may then, for example, become stiffer after insertion into the vein. The material may for example be a metal, including in the form of a metal alloy. Specifically, the material may be aluminum. Generally speaking, all materials that are mentioned in the section “General Material Properties” above may be envisioned.

The same effect may be achieved when using a material which has tumefying and/or hygroscopic properties. The material may for example be or contain bentonite, hydrogel or starch.

It is also conceivable that the turns of the spiral structure are configured to be internally hollow over a partial length or the predominant or entire length of the spiral structure. This cavity may contain a fluid or a gas/vapor which expands with increasing ambient temperatures, for example again in the event of a rise from room to body temperature. The cavity may be connected to at least one channel, to which an aspiration element may again be attached selectively, for example by means of a connector. The cavity of the spiral structure may therefore be filled by a user, for example with the aid of an aspiration element, with a fluid, gas or vapor. Likewise, the latter may in turn be evacuated from the cavity. Advantageously, a valve-like element may additionally be used, which prevents the fluid, gas or vapor from being able to flow out of the cavity via the channel when the aspiration element is not attached to the connector.

It is, however, also conceivable that the turns of the spiral structure are developed with a plurality of cavities, which are separated from one another by structures and thereby no longer communicate with one another. In such a case, these non-communicating cavities may be connected to different channels, to which an aspiration element may again be attached selectively, for example by means of a connector. Valve-like elements may advantageously be employed as just described.

The spiral structure may consist of at least one material which has resilient properties. The spiral structure may therefore expand or be compressed, that is to say change its volume, even independently of the existing ambient temperature, depending on whether or not it is filled with a fluid, gas or vapor, as just described.

The volume of the spiral structure may thus be controlled accurately according to the fill quantity. This may directly influence the properties of the venous catheter.

A spiral structure configured in this way may be located in the wall of a venous catheter and be delimited internally from the lumen of the venous catheter by a non-resilient layer. Thus, the outer diameter of the venous catheter increases when a fluid, gas or vapor is supplied, without the diameter of the lumen changing.

A spiral structure configured in this way may be located in the wall of a venous catheter and be delimited outward from further structures of the venous catheter or the vein itself by a non-resilient layer. Thus, the diameter of the lumen decreases when a fluid, gas or vapor is supplied, without the outer diameter of the venous catheter increasing.

This creates a venous catheter which can be adapted optimally to the individual vein profile and a vein diameter that also increases, for example in the direction of the heart.

A venous catheter developed as described above may also be used as a hose for further applications independently of an indwelling venous cannula. For example, it may be used as a hose for other catheter systems or as a breathing hose, for example including as an endotracheal tube. A puncture needle may also have the properties just described.

### Particular Version of the Venous Catheter in the Mechanically Loaded Skin Region

According to one advantageous configuration of the invention, it is provided that the outer tunic respectively has one or more reinforcing elements in one or more regions which are exposed to increased mechanical load. Such reinforcing elements may, for example, be configured as supporting structures.

Also conceivable is an accordion-like, spiral or undulating version of the venous catheter only at the sites with particular mechanical loading, for example where the venous catheter is located at skin level and merges remotely from the vein into further components of the indwelling venous cannula. Here, a particular design of the venous catheter may be advantageous over a distance of 1-20 millimeters, in particular over 2-10 millimeters. It may also be reinforced with further structures or elements only over this length. Over this length, the venous catheter may, for example, also be reinforced by further layers or a protective sleeve. This may enclose the venous catheter over a partial length in the form of a bowl, semicircularly or circularly. The venous catheter may be inseparably connected to this protective sleeve. It is, however, also conceivable for the venous catheter to be guided longitudinally displaceably and/or twistably/rotatably in the latter. The protective sleeve can prevent potential material damage to the venous catheter, for example breaking.

The formation of a very flexible connection in the region just mentioned is also possible. For instance, an additional connector may be used. Likewise, a fluid guiding element which continues in the direction of the vein into the venous catheter and can be twisted or angulated may be used. It is in this case possible that it can be twisted or angulated freely in all directions. Such a fluid-guiding element may also be configured in the manner of an articulation, for example in the manner of a ball or hinge articulation.

The venous catheter may also be guided in the region just mentioned through an articulation-like, for example ball or hinge articulation-like, structure that encloses it partially or wholly. The venous catheter may in this case be guided internally through this structure and be partially or fully enclosed by the structure.

The venous catheter may also be braced by a structure bearing on it externally, which may bear on it partially, predominantly or fully, so that it does not kink. The outer-lying structure may particularly be configured in the form of a bowl, semicircularly or circularly, and may also be part of an articulation-like structure as described above or be combined with such a structure.

Attachment elements may be connected to the other components of the indwelling venous cannula by means of the articulation-like structures described above. A fluid-guiding structure may be guided internally through these structures.

### Adjustable Flexibility of the Venous Catheter

According to one advantageous configuration of the invention, it is provided that the puncture device has at least one control element, with which the flexibility of at least one region of the outer tunic can be readjusted by the user.

A vasiform structure, which may be mounted longitudinally displaceably and twistably/rotatably about the longitudinal axis of the venous catheter in relation to the other wall constituents, may be introduced into the wall of the venous catheter, i.e. the outer tunic, in a cavity. The cavity may in this case be closed in the direction of the vein but open in the direction of the user. The vasiform structure, which may also be configured not fully circularly but, for example, also semicircularly, may protrude beyond the venous catheter in the longitudinal direction. In particular, it may protrude in the longitudinal direction beyond the venous catheter and an extension element located in the direction of the user, in which or in the wall of which it may likewise be located as just described. The structure may therefore be controlled in its position by a user.

The flexibility and stiffness of the venous catheter located in the blood vessel may thereby be varied individually by the user. This is relevant in particular when the venous catheter has not yet reached its definitive position in the vein. A new group of catheter systems is thereby created.

If the vasiform structure is also configured to be piercing-resistant, movements of the venous catheter in relation to the puncture needle are possible in all directions, even multiply in relation to one another, without the venous catheter being capable of being thereby damaged. The vasiform structure may be formed from at least one piercing-resistant material or be coated with at least one such piercing-resistant material. The piercing resistance of the material in this case refers to possible piercing of the venous catheter by the puncture needle, which is intended to be prevented by the piercing-resistant material. The piercing-resistant material may be a metal, including in the form of a metal alloy, or a correspondingly piercing-resistant plastic material or natural material. For example, carbon fiber-reinforced laminate materials, polymers and/or Teflon may be envisioned as plastic materials, including in combination with one another. Aramids may likewise be employed.

Because of the aforementioned properties of the indwelling venous cannula, it may however also be possible for a puncture needle withdrawn into the venous catheter initially to be left in the venous catheter in order to carry out a puncture again at a later time, for example of more deeply lying structures. A puncture needle withdrawn sufficiently far into the venous catheter is in this case enclosed by the outer-lying piercing-resistant venous catheter and cannot damage surrounding structures.

### Applicator at the Near-Vein Part of the Venous Catheter

According to one advantageous configuration of the invention, it is provided that the puncture device has an applicator at the end near to the hollow body for the application of a substance in the region of the entry site of the puncture needle on the hollow body. The applicator may for example be configured as a liquid-tight hollow body, for example as a pouch, which is filled with an antimicrobial liquid or another fluid. The antimicrobial liquid may for example be an alcoholic liquid, a liquid containing iodine or another disinfecting liquid. The fluid may for example have friction-reducing, antimicrobial, local anesthetic and/or anticoagulant properties. For example, the applicator may also contain a cooling medium, for example cooling liquid and/or ice, in order to cool the region of the entry site of the puncture needle. The application of the puncture device may thereby be made relatively painless for living beings. The applicator may, in particular, be configured in such a way that it cannot penetrate the tunic of the hollow body to be punctured, i.e. it is meant to be configured not sharply but rather bluntly in the region near to the hollow body.

A further structure, i.e. the applicator, which partially or fully encloses the venous catheter and/or the puncture needle before insertion into the patient, may be present. This structure may be a closed hollow body which is longitudinally displaceable in relation to the venous catheter. It may for example be cylindrical with a round or ellipsoidal cross-sectional area, including in the form of a rod, a nozzle or a tube. It may also be configured in the form of a cuboid. It may for example likewise be developed in the form of an applicator, sponge, foam ring or gel ring, a bag or a capsule. It may also be configured in the form of a disk, brim or ramp.

The structure may also be configured so that at least a part of the hollow body is located before the puncture needle tip in the longitudinal direction. It is then advantageous for the hollow body to be surrounded by a wall that can be pierced by the puncture needle tip. This wall may also be a membrane. If the hollow body then contains a fluid, the puncture needle and the venous catheter are wetted with the latter before insertion into the vein. The fluid may for example have friction-reducing, antimicrobial, local anesthetic and/or anticoagulant properties.

A structure which is located in front of the puncture needle tip before use of the indwelling venous cannula also has the advantage that it provides pricking protection for the patient and the user until it is placed onto the skin of the patient.

In one advantageous development, the outer diameter of a structure enclosing the venous catheter is at least 1.5 times as great as the outer diameter of the venous catheter. In a further advantageous development, the outer diameter is at least 2 times as great as the outer diameter of the indwelling venous catheter. In further advantageous developments, the outer diameter of the structure is at least 3 times, at least 4 times, at least 5 times and at least 6 times as great as the outer diameter of the indwelling venous catheter.

The structure enclosing the venous catheter may taper conically toward the end near to the hollow body, for example at an angle of at least 30 degrees, 45 degrees, 60 degrees, 75 degrees or 90 degrees, based on a 360 degree system. The angle refers to the angle measurement between two outer-lying oblique dilatation faces of the structure, and not to the midaxis of the structure.

Thus, when the puncture needle and the venous catheter are inserted through the skin, the structure cannot penetrate the latter. By its longitudinal displaceability, the venous catheter is wetted with the aforementioned fluid over its predominant or entire length. After the wetting process, the structure may as one option remain on the venous catheter and at least partially enclose the latter protectively, for example in the region of the vein-remote transition into further components of the indwelling venous cannula. The structure may be a kind of “tunic” which is initially located “forward” on the venous catheter and then “slips back” on the latter during the insertion of the venous catheter (while wetting it) and then protectively encloses the entry region of the venous catheter into the skin (minimization of the infection risk).

It is, however, also conceivable that the structure is removable from the venous catheter and, for example, is configured to be cleavable or tearable.

The structure may additionally be provided with an adhering or adhesive surface, in order to stabilize the indwelling venous cannula on the skin during puncture.

It is likewise conceivable that the puncture needle and/or the venous catheter are already fed through the aforementioned structure even before the puncture process has begun. The structure may thus partially or fully surround the puncture needle and/or the venous catheter. The wall of the cavity of the structure may be fitted longitudinally displaceably and sealing in a fluid-tight manner on the puncture needle and/or the venous catheter.

It is also conceivable that the cavity of the structure is enclosed at least partially by at least one further cavity, which is delimited from the first cavity by an additional wall. Likewise, at least one further cavity may be adjacent to the first and likewise be delimited therefrom by a wall. The venous catheter may thus be wetted by different fluids which, although they may be used compatibly on the venous catheter, need to be stored separately from one another before use.

It is also conceivable that the first cavity contains urea and the other cavity contains water. Other substances that are used in commercially available instant cold packs may also be employed. In this way, the puncture needle and/or the venous catheter may be cooled before and during the introduction into the patient, and the puncture needle and/or venous catheter may thus be inserted relatively painlessly into the anatomical structures.

The cavities may respectively also contain a capillary-, pore- and/or honeycomb-like structure, for example including in the form of a foam, which has direct contact with the venous catheter and can ensure both wetting of the venous catheter with a fluid and mechanical cleaning and/or drying of the venous catheter. It is also conceivable that at least one cavity of the applicator can be filled with a fluid from the outside, for example including through a bacterial filter. The aforementioned structure may then ensure uniform filling of the cavity with a fluid, and likewise continuous release of the fluid onto the venous catheter.

### Version of the Venous Catheter as a Pigtail Catheter

According to one advantageous configuration of the invention, it is provided that the puncture device has a mechanism by which the outer tunic can be converted into a spiral configuration by a relative movement in relation to the outer tunic. In this case, the outer tunic may initially have a non-spiral configuration, for example an elongate, linear configuration. The puncture needle may in this case be guided through an outer tunic configured in such a way. By withdrawing the puncture needle in relation to the outer tunic and/or pushing of the outer tunic in relation to the puncture needle, the outer tunic may be converted into the spiral configuration.

The venous catheter may also be configured to be curled at the immediate near-vein end. In contrast to pigtail catheters used in medicine, however, the catheter is initially stretched not by an inner-lying guide wire but by the inner-lying puncture needle. As soon as the venous catheter has been pushed forward beyond the tip of the puncture needle in the direction of the vein or the puncture needle is withdrawn from the venous catheter, it transitions from a straight profile into a curled or at least bent profile. The venous catheter may for example consist of silicone or polyurethane, or alternatively may contain at least one shape-memory alloy, for example nickel-titanium or nickel-titanium-copper. Combinations of copper, aluminum, nickel and zinc may also be envisioned. The version having at least one shape-memory alloy may also exist only in the region of the venous catheter that is intended to transition into a curled or at least bent profile. This region of the venous catheter may also contain a spiral or undulating structure. It is, however, also possible that a spiral or undulating structure is no longer present in this region of the venous catheter but forms the region of the venous catheter that remains straight.

Recesses/openings may be arranged over the entire length of the venous catheter, but preferentially also at the site at which the angulating or bending of the venous catheter is maximal. For example, liquids to be infused thereby continue to follow the flow direction away from the venous catheter into the blood vessel.

An indwelling venous cannula developed in such a way may be used particularly in the field of urology, or alternatively for pneumothorax drainage, and particularly for premature/newborn babies.

### Communication of Lumens Inside the Venous Catheter

According to one advantageous configuration of the invention, it is provided that the outer tunic is configured with multiple lumens, two or more lumens being connectable to one another via one or more openings. Such different lumens are normally separated from one another. By such openings, however, fluid communication between two or more lumens may be allowed in certain sites.

In an at least two-lumen version of the indwelling venous cannula, the separation between the lumens may be partially or predominantly negated in particular within the venous catheter. This may be done by recesses which are introduced into the structures that separate the lumens from one another. These recesses may, for example, have a circular or ellipsoidal structure and also be provided with flap-like structures that allow the flow of fluid only in one direction. Furthermore, the recesses may contain elements that fulfill the function of overflow valves.

The recesses may likewise be with filter-like structures that allow only certain fluid constituents to pass through the recesses. It is also conceivable that at least one lumen merges partially or fully into another inside the venous catheter. A lumen may also end blindly and represent a kind of cul-de-sac or reservoir, for example for a medication. The blindly ending lumen may likewise contain at least one sensor.

It is also conceivable that the structures which separate the lumens from one another inside the venous catheter contain externally controllable elements, by means of which it is possible to influence whether the fluids in the initially separated lumens can communicate with one another. This may also be done such that at least two lumens inside the venous catheter are twistable about the longitudinal axis or displaceable in the longitudinal direction and contain mutually complementary recesses. If these recesses are then brought above one another by corresponding movements, the fluids can communicate between the different lumens; otherwise, the fluids are strictly separated from one another.

It is thus also possible at any time to connect to one lumen up to at least one further lumen inside the venous catheter, and therefore when necessary to increase the flow rate when this is essential rather than, for instance, the presence of an at least second separate lumen.

The separated lumens may be separated from one another by at least one wall that has a spiral/undulating or network-, braid- or grid-like surface and developed with a sealing coating, for example a PTFE coating. A combination with transversely, longitudinally or diagonally running structures may be envisioned, and these may also by themselves ensure a sufficient stability of the lumen wall. In one advantageous development, the aforementioned recesses are enclosed by structures, for example annular structures, which may also be configured as wire structures, that stabilize the shape of the recesses and keep them open.

A venous catheter configured in such a way may also be used independently of an indwelling venous cannula, for example as a midline catheter, central venous catheter (CVC) or dialysis catheter.

### Automatic Forward Feed Mechanism of the Venous Catheter

According to one advantageous configuration of the invention, it is provided that the puncture device has an automatic forward feed mechanism by which, if appropriate as a function of at least one external condition, an automatic forward feed of the outer tunic into the punctured hollow body through the opening generated by means of the puncture needle is generated.

In this case, there may be a mechanism which ensures that in the event of a retrograde flow through the puncture needle or the venous catheter with the blood of the patient during the puncture process, the venous catheter is automatically pushed forward in the direction of the vein beyond the puncture needle. The mechanism may, for example, be a spring mechanism or contain such a mechanism.

It is conceivable that the spring mechanism is dissolved by the heat of a fluid flowing back in the direction of the user through the venous catheter, for example blood, or alternatively by the heat of a gas or vapor. A fluid-guiding component of the indwelling venous cannula, for example the venous catheter itself or a hollow extension element following on from the venous catheter in the direction of the user, or the chamber 7, may be formed from a bimetal or contain such a bimetal.

This bimetal may be coupled to a path limiting element which is in contact with a path limiting element of the venous catheter and prevents the venous catheter from being pushed forward in the direction of the vein in the unheated passive state.

If a warmed fluid, for example blood, then flows in the vicinity of the bimetal, the latter bends. It may in this case bend such that the path limiting element of the bimetal is moved away from the path limiting element of the venous catheter.

If the venous catheter then has the natural tendency to be pushed forward in the direction of the vein because of a spring coupled to it, precisely this effect then takes place. The pushing forward takes place more slowly or more rapidly, depending on the pretensioning of the spring. The pushing forward may also be modified in its speed or fully prevented by a further manual actuation element.

In the case of pushing forward slowly, fragile veins, for example, may be punctured gently. The advantage is further that an indwelling venous cannula configured with the mechanisms just described may be operated with one hand. In addition, inadvertent movements of the indwelling venous cannula by the user may be substantially prevented. They may concentrate fully on the stabilization of the indwelling venous cannula on the patient. This minimizes the risk that the necessary components of the indwelling venous cannula, in particular the puncture needle tip and the venous catheter, become dislocated from the vein again during the puncture process since few manipulations then need to be carried out on the indwelling venous cannula in this critical phase.

Instead of the aforementioned bimetal, at least one material that experiences a volume change, for example a volume increase, for example by tumefication, upon fluid/blood contact may also trigger the effects just described.

Is also conceivable that a tensioned spring of at least one structure, which consists of at least one resorbable material, is kept under tension and therefore in its position. This spring may by way of example be located between the puncture needle and the venous catheter, and may connect them to one another. This spring may, however, also be connected only to the puncture needle or only to the venous catheter. It is also conceivable, however, that the spring is located not firmly connected to the puncture needle or the venous catheter in a vein-remote section of the indwelling venous cannula, and may push either the puncture needle or the venous catheter in the direction of the vein in the event of a state change.

If blood or another fluid then flows into the venous catheter, the resorbable structure is dissolved and the spring experiences a state change, for example an increase of its length. The spring then ensures pushing of the venous catheter forward in the direction of the vein.

If blood flows back through components of the indwelling venous cannula, a flap, valve or click mechanism which ensures that the force is deviated or controlled so that the venous catheter is pushed forward therefrom in the longitudinal direction beyond the puncture needle in the direction of the vein may also be activated. This may for example also be relevant in the case of puncturing arteries, in which a higher pressure prevails than in veins.

It is, however, also possible that the puncture needle itself is pushed away from the vein in the direction of the user by a return flow of blood into the puncture needle and/or the venous catheter in the longitudinal direction. For this purpose, the puncture needle may advantageously be developed internally where it is hollow, that is to say it has its lumen, with flap-, wing- or valve-like elements or other elements that increase the flow resistance. These may also be controllable in the sense that they are set protruding into the lumen, i.e. moved away from the wall of the lumen, by the blood flowing back into the puncture needle. Normally, the outer-lying venous catheter is pushed forward beyond the inner-lying puncture needle into the vein. In this case, the indwelling venous cannula is often operated with 2 hands and unavoidable “jerking” of the indwelling venous cannula takes place. This “jerking” may cause slipping of the venous catheter out of the vein, and above all in the case of small veins already endanger the puncture outcome. This may be avoided by the configuration of the puncture device as described above.

If a blood flow out of the vein then impinges on these structures, the puncture needle is pushed away from the vein in the direction of the user if they are not holding it firmly. So that they do not need to do this when inserting the indwelling venous cannula into the patient, further fastening and holding elements may advantageously be fitted on or to the indwelling venous cannula.

In principle, these elements may also be located on the outer side of the puncture needle or on the inner side of the venous catheter enclosing the puncture needle. In the latter case, it may be advantageous for them to interact with further elements on the outer side of the puncture needle.

The venous catheter or puncture needle may also be passively washed into the vein by the blood flow. In this case, it may be advantageous that they are then not connected to further components of the indwelling venous cannula. It is also conceivable that a negative venous pressure potentially present in many veins leads to the venous catheter also being drawn into the vein in the longitudinal direction. The advantage is that in the intended venous location of the venous catheter, inadvertent introduction of the latter into an artery is then no longer possible since a higher pressure prevails there.

Washing inward may also be initiated when the blood of the patient flows through the hollow extension element described in PCT/EP2019/057097. In this case, it is possible for this blood flow to be accelerated by a reduced-pressure element. A vacuum effect may be used for this purpose.

Prevention of Contamination of the Indwelling Venous Cannula when Attaching a Syringe or Infusion Line

According to one advantageous configuration of the invention, it is provided that the puncture device has a contamination protection device for protecting the end of the puncture device remote from the hollow body against contaminations. In particular, connectors for attaching further elements, for example hoses, to the puncture device may thereby be protected against contaminations. The contamination protection device may, for example, have an outer protective structure.

When attaching an aspiration element or further elements, via which the fluids or medications can be delivered, it is in any case necessary to avoid contamination of the indwelling venous cannula with germs, for example by the hand of the user or the skin of the patient, in the region of the fluid-guiding element (in what follows, only “element”) on the chamber 7 and/or the attachment element 9.

This may be achieved by a vein-remote fluid-guiding, for example cylindrical, element that is located outside the patient being respectively enclosed partially or fully by at least one further protecting structure. This outer protecting structure projects in the longitudinal direction beyond the fluid-guiding element in the direction of the aspiration element. In one advantageous configuration, the outer protecting structure projects beyond the fluid-guiding element in the longitudinal direction by 1-5 millimeters, although it may also project beyond the fluid-guiding element by 6-10 millimeters or even by more than 10 mm in the longitudinal direction. The fluid-guiding element may, for example, also be configured in the form of a three-way stopcock.

The fluid-guiding element may be guided longitudinally displaceably through the outer protecting structure. Likewise, the outer protecting structure may only be fitted longitudinally displaceably on the fluid-guiding element or surround the latter partially or fully.

The fluid-guiding element may be guided twistably about the longitudinal axis through the outer protecting structure. Likewise, the outer protecting structure may only be fitted twistably about the longitudinal axis on the fluid-guiding element or surround the latter partially or fully.

Also conceivable, however, is a differently configured double structure consisting of a fluid-guiding element and an outer protecting structure. For instance, various geometrical shapes in regard to the respective inner or outer diameters or the cross-sectional areas may be combined. Respectively ramp-shaped structures or structures that ensure deliberate and reversible wedging of the fluid-guiding element with the outer protecting structure may also be envisioned. Latching into one another or a click-clip mechanism may also be envisioned.

Screw thread-like structures, which can engage with one another or fundamentally interact with one another, may also be formed on the outer side of the fluid-guiding element and/or the inner side of the outer protecting structure. Friction-increasing, tacky or ribbed structures may be present on the outer side of the outer protecting structure, so that a user can safely operate the outer protecting structure without slipping.

A twistability of the fluid-guiding element in relation to the outer protecting structure about the longitudinal axes without a substantial longitudinal displacement may likewise be achieved by furrow-shaped recesses that are straight in the direction of the transverse axis, into which elevations on the inner side of the outer protecting structure engage, being formed on the outer side of the fluid-guiding element. Alternatively, these straight furrow-shaped recesses may also be formed on the inner side of the protecting outer structure and the elevations just described may also be formed on the outer side of the fluid-guiding element.

At least one path-limiting element may be fitted on the fluid-guiding element or the outer protecting structure or on both. The path-limiting elements may in this case engage in one another or interact with one another in another way. These path-limiting elements may influence, in particular reduce, allow up to a certain point or make impossible, both the displacement of the fluid-guiding element or the outer protecting structure individually or in relation to one another in the longitudinal direction. They may likewise be configured so that they influence, in particular reduce, allow up to a certain point or make impossible, a twisting movement about the longitudinal axis.

There may be a spring between the fluid-guiding element and the outer protecting structure. In a longer (less compressed) state, this may ensure that the outer protecting structure projects in the longitudinal direction beyond the fluid-guiding element in the direction of the user. If the spring is then compressed by an active influence, the outer protecting structure is also displaced in the direction of the vein and therefore no longer projects beyond the vein-remote end of the fluid guiding element. Another suitable element may thereby be connected to the fluid-guiding element. If this is then removed, there is once more protection of the vein-remote end of the fluid-guiding element since the outer protecting structure again projects beyond it in the longitudinal direction. The spring may also be located between the outer protecting structure and the vein-remote end of the chamber of the indwelling venous cannula.

It is also conceivable that the site, for example of an aspiration element, an infusion line, a three-way stopcock or another element of a stopcock system, which comes in contact with the vein-remote end, is configured similarly or identically as just mentioned. In order to allow connection to the vein-remote end configured in this way of the fluid-guiding element of the indwelling venous cannula together with the outer protecting structure, the outer protecting structure of the aspiration element may have a larger diameter than the outer protecting structure of the fluid-guiding element of the indwelling venous cannula. It therefore slides over the outer protecting structure of the indwelling venous cannula during the connection of the indwelling venous cannula and aspiration element. It is also conceivable that the outer protecting structure of the aspiration element has a smaller diameter than the outer protecting structure of the fluid-guiding element of the indwelling venous cannula. In such a case, the outer protecting structure of the aspiration element may slide under the outer protecting structure of the indwelling venous cannula during the connection of the indwelling venous cannula and the aspiration element. In one advantageous version, the outer protecting structure of the aspiration element may slide into a cavity which is formed on the indwelling venous cannula between the outer side of the fluid-guiding element and the inner side of the outer protecting structure of the indwelling venous cannula.

Connection sites of infusion lines or other hose systems, for example breathing hose systems, may also be configured in the manner described, independently of an indwelling venous cannula, as may connecting sites from and to medical filter systems, for example bacterial filter systems. Known Luer lock elements may also advantageously be developed in the manner described. In general, use independently of an indwelling venous cannula may be carried out.

A semicircular or bowl-shaped structure, which is open toward the top and protects the vein-remote attachment of the chamber against contamination by skin germs, may likewise be fitted at the vein-remote end of the chamber 7 and/or of the attachment element 9. This structure may be configured so that it is pliable or deformable and can be both shaped by the user or the patient and adapted to the individual anatomical features. The structure may likewise have thermoplastic or antimicrobial properties and be provided with a friction-increasing or tacky surface, in order to reduce the risk of the indwelling venous cannula slipping out of the patient.

### Spring Retention Mechanism as Pricking Protection

The indwelling venous cannula may advantageously be developed with a spring retention mechanism for retaining the puncture needle in the venous catheter.

This spring retention mechanism may be configured so that a spring-like or spiral structure (in what follows, “spring”) in the initial/passive state ensures that the entire puncture needle, particularly including the near-vein end of the puncture needle and the puncture needle tip, is enclosed by the venous catheter. The patient and the user are thus protected against needle injuries. The spring is in this case in its relaxed, deactivated passive or initial state, and in the case of a compression spring is configured to be longer than in the activated state, or in the case of a tension spring is configured to be shorter than in the activated state. At the transition into the activated state, i.e. when the puncture needle is intended to be pushed out of the venous catheter at the near-vein end, the spring force then needs to be overcome by the user.

The spring may in this case be configured so that it encloses the puncture needle over the partial length, predominant or entire length of the latter, that is to say the puncture needle is at least partially guided longitudinally displaceably and/or twistably through the spring.

In one advantageous configuration, the outer diameter of the puncture needle may be different, for example less, over at least a partial length over which the spring encloses the puncture needle than the other outer diameter of the puncture needle.

The longitudinal displaceability of the spring may be reduced or negated by at least one path limiting element that may be located on the puncture needle. The path limiting element may be configured so that the puncture needle has an alternating diameter, for example including in the form of elevations, edges, projections, wings or arches, by which the spring enclosing the puncture needle is fixed in a determined position. These may be formed at one or both ends of the spring, or alternatively between the spiral spring turns that are present. It is also possible that the diameter of the puncture needle increases in the form of a ramp or stepwise.

Because the longitudinal displaceability of the spring in relation to the puncture needle is reduced or negated as described above by at least one path limiting element, the spring prevents spontaneous displacement of the puncture needle in relation to the venous catheter in the longitudinal direction. The spring must be activated, for example squeezed/compressed, by an external influence in order to achieve in particular a longitudinal displacement of the puncture needle in the direction of the vein, which is necessary so that the puncture needle tip can project beyond the venous catheter in the direction of the vein in the longitudinal direction. Only in this way can a puncture be carried out.

The spring may also be integrated directly into the puncture needle or connected to the puncture needle. The spring may, for example, be connected to the puncture needle such that it follows on from the puncture needle remotely from the vein. The spring may also emerge from the puncture needle remotely from the vein, that is to say it may be connected inseparably to the puncture needle. In a further advantageous development, the spring may be located in a hollow extension element, which constitutes the continuation of the venous catheter in the direction of the user and to which, for example, an aspiration element or an infusion hose may be attached. In a further advantageous configuration, the spring may enclose the puncture needle in the region where the puncture needle is connected remotely from the vein to further components of the indwelling venous cannula and/or merges into the latter.

The spiral spring turns of the spring may be spaced apart from one another less at the two ends in the direction of the longitudinal axis of the spring than between the ends. A spacing that alternates over the entire length of the spring is possible.

The puncture needle may likewise be coupled or connected to an element which has contact with a spring, or may be enclosed by such an element. This element may, for example, be configured in the form of a disk-like structure having a central opening, in which the puncture needle is guided displaceably and/or also twistably. It is, however, also conceivable that this element is firmly connected to the puncture needle and there is therefore no longitudinal displaceability of the element in relation to the puncture needle.

If the puncture process is then begun, the user may activate the spring retention mechanism and therefore displace the puncture needle along its longitudinal axis in the direction of the vein. The near-vein end of the puncture needle, in particular the puncture needle tip, then projects in the longitudinal direction in the direction of the vein beyond the venous catheter. Puncture of the vein is therefore made possible.

A force by the user always needs to act externally in the longitudinal direction on the spring so that the latter is activated and the tip of the puncture needle can be pushed in the longitudinal direction in the direction of the vein beyond the venous catheter. If no force then acts externally, the puncture needle is automatically withdrawn by the spring into the venous catheter and therefore deactivated, and there is pricking protection since the entire puncture needle is enclosed by the venous catheter. If a force acts only temporarily, the pricking protection is negated only during this time.

The spring retention mechanism may, for example, also be activated by a near-user actuation element, for example a button or stamp (in what follows, “button”), which may be pressed downward and triggers the displacement movement of the puncture needle, for example in the manner of a ballpoint pen mechanism.

In one advantageous development, the puncture needle may be displaced in the longitudinal direction in the direction of the vein the first time the button is pressed downward, and then initially remain in this position, for example by latching or another interaction with a path limiting element. The button may also remain in the pressed-down position, for example by latching or another interaction with a path limiting element. At least one path limiting element may thus respectively ensure that the puncture needle and/or button remain in the desired position.

When the button is pressed downward for a second time, the puncture needle and the button return into their respective initial position, and the puncture needle is thus then displaced away from the vein. In this case, a state in which the puncture needle and the button are displaced even more in the longitudinal direction in the direction of the vein than before can temporarily be achieved.

The button may be directly connected to the puncture needle, may continue the latter for example at the vein-remote end of the indwelling venous cannula, or for example enclose it. It is also conceivable that the button is connected to the puncture needle only indirectly by means of a further component, for example a further spring.

Once the puncture of the vein is achieved, which is revealed by a return flow of blood into components of the indwelling venous cannula, the spring retention mechanism initially activated for the puncture is deactivated by the user, and the puncture needle is displaced away from the vein in the longitudinal direction. The venous catheter may then be pushed forward into the vein without the tip of the puncture needle projecting beyond the venous catheter in the longitudinal direction in the direction of the vein. Yet since the puncture needle continues to be located in the vein catheter, it can brace and thereby stabilize the latter for the pushing forward into the vein.

The spring retention mechanism may fundamentally be activated or deactivated by pressure or traction on an attachment element or holding element which is located remotely from the vein on the indwelling venous cannula. In particular, both activation and deactivation by pressure are possible. It may, in particular, be advantageous that the spring can remain in the activated state without a force acting from the outside being continuously required for this. A mechanism which ensures latching of the spring in the activated state may be used.

In one advantageous configuration, an attachment element located on the upper side of the indwelling venous cannula, to which for example an aspiration element may be attached, may also be connected to the puncture needle by means of a spring. The attachment element may in this case be connected directly or by means of a further element to a spring. It is then possible that, for example, the user can vary the location of the puncture needle in the longitudinal direction with one finger of the puncturing hand. This holding element may have a complementary shape or a negative contour of a human thumb or a human fingertip. The surface may consist of at least one friction-increasing material or be coated with at least one such material.

In this case, it is conceivable that because of special properties, for example because of a different number and/or spacing of the turns and/or resilient properties of the spring, the distance by which the tip of the puncture needle, that is to say the near-vein end of the puncture needle, projects beyond the venous catheter in the longitudinal direction, can be adjusted before the puncture process. By the properties just mentioned, the maximum mobility of the puncture needle in the longitudinal direction, and in particular also the maximum forward feed in the direction of the vein, may also be predefined.

By the properties just mentioned, it is also possible to predefine a maximum pressure that can be exerted onto the puncture needle or the venous catheter so that inadvertent traumatization and possibly second perforation of the venous wall can no longer occur in particular also when the venous catheter is being pushed forward into the vein.

A traffic light system with the colors “green”, “yellow” and “red” may be envisioned here. It is also conceivable that the forward feed of the components to be pushed forward is automatically terminated or made impossible in the direction of the vein in the red range.

In one advantageous development, pricking protection may also be configured so that there is an invertible structure or an invertible element (in what follows, “invertible element”) on the inner side of the venous catheter or on the inner side of a component that is connected to the venous catheter in the longitudinal direction in the direction of the vein.

This invertible element may for example be configured in the form of a hose tapering or widening in diameter, in the shape of a trumpet or in a shape resembling a brimmed hat. When the near-vein tip of the puncture needle then moves away from the vein, this element folds over as the tip passes and encloses the latter protectively and permanently.

The inner side of the invertible element may advantageously consist of a friction-increasing material or be coated with such a material. The invertible element may be formed from at least one piercing-resistant material. It may also have spiral, undulating and/or braid-like structures.

The piercing-resistant material may be a metal, including in the form of a metal alloy, or a correspondingly piercing-resistant plastic material or natural material. For example, carbon fiber-reinforced laminate materials, polymers and/or Teflon may be envisioned as plastic materials, including in combination with one another. Aramids may also advantageously be used. The invertible element may also be formed from nitinol. All materials mentioned in this document may be used, individually or in any desired combination with one another.

It is also conceivable that the element just described is not inverted, but during a relative movement of the puncture needle in relation to the venous catheter is brushed/pushed over the puncture needle tip. If the element is configured in at least two plies, at least one layer of the element may also be brushed/pushed by a rolling movement over the puncture needle tip.

### Multilayer Puncture Needle as Pricking Protection

Pricking protection may also be configured such that the puncture needle has or consists of two hollow bodies, which are mounted longitudinally displaceably in relation to one another. These hollow bodies may for example be formed from a metal or a metal alloy, or alternatively of any other piercing-resistant material as described above.

The outer hollow body may be configured cylindrically and in the shape of a stamp at both ends. The inner hollow body, which is guided in the outer hollow body, may likewise be configured cylindrically but have a smaller outer diameter in relation to the outer hollow body.

The inner hollow body may be provided at its near-vein end with a sharp structure and/or with a point suitable for puncturing, and thus form the puncture needle tip.

The spring retention mechanism with a compression or tension spring, explained above in relation to the interaction between the puncture needle and the venous catheter, may alternatively or additionally also be present in relation to the interaction between the inner and outer hollow bodies of the puncture needle. The spring retention mechanism is then used to retain the inner hollow body in the outer hollow body of the puncture needle. This spring retention mechanism may be configured so that a spring-like or spiral structure (in what follows “spring”) in the initial/passive state ensures by a spring force that the near-vein sharp structure of the inner hollow body adopts a position such that the outer hollow body projects beyond the inner hollow body together with the sharp structure, or at least receives it within itself, and in particular ensures that this position does not change without a pressure from the outside. At the vein-remote end, the inner hollow body may for example be provided with a spring or enclosed by a spring. A holding element or a connector for an aspiration element may be formed at the vein-remote end of the inner hollow body.

If the inner hollow body is then manually pressed actively in the direction of the vein, its near-vein tip projects beyond the outer hollow body in the direction of the vein and may be used for vein puncture. The vein-remote spring is in this case actively tensioned (compressed/shortened). If the manual pressure is then released, the spring ensures that the inner hollow body is moved back away from the vein, that is to say its tip is again enclosed by the outer hollow body. In this way, pricking protection is always provided outside the direct puncture process.

The inner and outer hollow bodies may be mounted both longitudinally displaceably and twistably about the longitudinal axis, including respectively in relation to one another, in a venous catheter. The movements, including in relation to one another, may in this case be reduced or negated by at least one path limiting element.

Between the inner and outer hollow bodies, there may be at least one material or substance which reduces the frictional resistance between these two bodies. These may for example be germ-free fat-like or oil-like substances or alternatively nanoparticles. The same may apply for the space between the outer hollow body and the venous catheter. Closure elements may ensure that these substances cannot leave the respective space, that is to say they are sealed fluid-impermeably on all sides. The space between the inner and outer hollow bodies or the space between the outer hollow body and the venous catheter may also be configured as a ball bearing or other rolling bearing.

Nonlinear Version of the Puncture Needle and/or of the Venous Catheter

Because of the special constitution of the venous catheter, the risk of damage thereto is minimized, including by a puncture needle that is not straight. For instance, besides the puncture needle, the venous catheter may also be bent, angulated or configured in the form of a spiral spring/screw before, during or after use on the patient.

It is likewise possible that the puncture needle and/or venous catheter contains straight sections between bent, angulated or spiral spring-/screw-like sections. It is likewise possible that bent, angulated or spiral spring-/screw-like sections may be combined with one another in any desired way. In one advantageous development, the venous catheter consists of at least one material which ensures that the venous catheter preserves its original shape over a partial length or its predominant or entire length even without the inner-lying puncture needle.

In one version suitable for this, the parts of the puncture system that are to be introduced into the patient may be introduced into the patient by means of a twisting movement, in a similar way as when screwing in a screw. The rotation axis in this case runs orthogonally to the skin surface.

In conventional blood sugar measurement apparatuses which have a sensor under the skin, a straight needle that goes under the skin is used. Then, however, the sensor can easily slip out of the skin. Damage may also occur if the straight needle pierces too deeply into further anatomical structures because of excessive pressure from the outside. A spiral needle, for example, may easily remedy such problems.

Likewise, the entire indwelling venous cannula may be configured to be bent, angulated or configured in the form of a spiral spring/screw. It is, however, also possible that in particular the vein-remote components of the indwelling venous cannula are still configured to be straight.

At its near-vein end, a puncture needle and/or a venous catheter configured as described above may contain a sensor that measures parameters in the blood or under the skin, for example in the subcutaneous adipose tissue. For example, the glucose value may be measured. For example, the oxygen partial pressure may also be measured. This sensor may be configured so that it allows data transmission to a receiver apparatus outside the patient, for example a smartphone or another electronic apparatus. The sensor may constitute both part of the puncture needle and/or of the venous catheter and be located on the inner or outer surface of the puncture needle and/or of the venous catheter. The sensor may for example be configured in the form of a spiral or in the manner of a film, other configurations being possible. The design just described may also be configured for extended or permanent implantation, for example directly under the skin. The design just described may, for example, also be used as a measuring instrument for measuring the glucose value in diabetic patients. The puncture needle and/or venous catheter may in this case be configured to be hollow, partially solid or fully solid, and additionally connected to a pump, for example an insulin pump, including via further connecting elements, for example hoses. The design just described need no longer in this case contain a venous catheter, and a puncture needle may have reduced measurements in respect of length and/or outer diameter and be configured more in the sense of conventional subcutaneous needles.

Angulations of the Puncture Needle and/or of the Venous Catheter in the Region of the Skin Entry Site

The puncture needle and/or venous catheter may be bent and/or angulated, particularly in the region of the vein-remote transition into further components of the indwelling venous cannula. With a venous catheter lying in the patient, the bending or angulation site corresponds to the region of the skin level. There, the venous catheter may be exposed to particular mechanical loads because of the way in which it is used. In contrast to a venous catheter running straight out of the indwelling venous cannula in the direction of the vein, it may project by at least 1° from its straight line, but preferably at an angle of from 2 to 5°, 6 to 10°, 11 to 25°, 26 to 45° or more than 45°. The venous catheter therefore forms a kink, which may be directed upward or downward. The venous catheter may also be developed in the aforementioned region with at least one spiral/undulating or network-, braid- or grid-like structure and with a sealing coating, for example a PTFE coating. A combination with transversely, longitudinally or diagonally running structures may be envisioned, and these may also ensure a desired stability of the wall of the venous catheter in this region on their own. The venous catheter may, however, also be configured as a conventional venous catheter in the aforementioned region and additionally contain the elements just mentioned. These may, in particular, also be introduced into the wall of a conventional venous catheter.

The puncture needle guided in the venous catheter may be developed in the same way.

### Adjustable Rigidity of the Outer Tunic

According to one advantageous configuration of the invention, it is provided that the outer tunic has two hollow bodies, which are mounted longitudinally displaceably in relation to one another. The two hollow bodies may, for example, be configured as an inner tube and an outer tube, in which the inner tube is at least partially arranged and in which it is displaceable. For example, there may be a displacement mechanism by which the extent by which the inner tube is displaced in relation to the outer tube can be adjusted. In this way, in particular, the extent of the overlap between the inner tube and the outer tube may be adjusted, for example by manual readjustment. If the extent of the overlap is adjusted to a low value, the rigidity is reduced and the outer tunic is therefore more resilient. If the extent of the overlap is adjusted to a high value, the rigidity is increased and the outer tunic is therefore less resilient. This functionality may, for example, advantageously be used when applying an indwelling venous cannula on a patient. During the application process, i.e. when pushing forward into the vein, it is advantageous for the rigidity of the outer tunic to be adjusted to a higher extent, i.e. the outer tunic is more rigid. In the inserted state of the indwelling venous cannula, i.e. when remaining in the patient, a lower rigidity is in turn advantageous because the venous catheter can then be adapted better to the blood vessel and possible movements of the patient.

### Side Holes

According to one advantageous configuration of the invention, it is provided that the outer tunic has lateral passage openings (side holes), through which liquid can flow from the inner side to the outer side of the outer tunic and vice versa. In particular cases, an improved medication delivery may thereby be achieved, in particular an improved distribution of the medication. It is also possible that some or all passage openings are closed initially, i.e. in the delivery state of the indwelling venous cannula, and only open in the state applied on the patient. For example, some or all passage openings may initially be sealed by a bioresorbable material. If the bioresorbable material then dissolves, the passage openings are uncovered.

The invention will be explained in more detail below with the aid of exemplary embodiments with the use of drawings, in which:

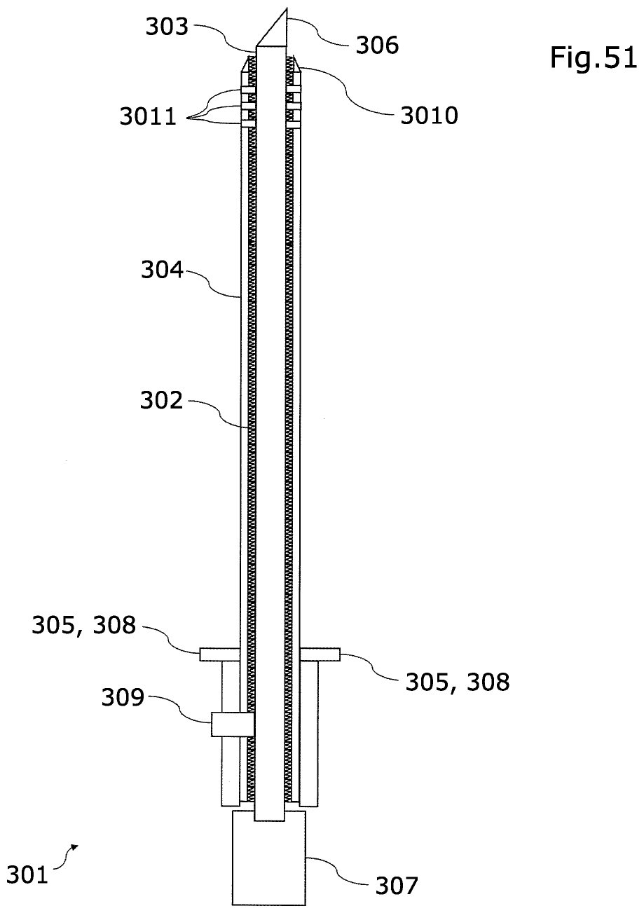

FIG. 51 shows a schematic representation of an indwelling venous cannula in a first embodiment in a longitudinal section,

FIG. 52 shows a schematic representation of an indwelling venous cannula in a second embodiment in a cross section,

FIG. 53 shows a schematic representation of the indwelling venous cannula according to FIG. 52 in a longitudinal section,

FIG. 54 shows a schematic representation of an indwelling venous cannula in a third embodiment in a cross section,

FIG. 55, 56 show schematic representations of a detail of the wall of an indwelling venous cannula according to FIG. 54 in a longitudinal section,

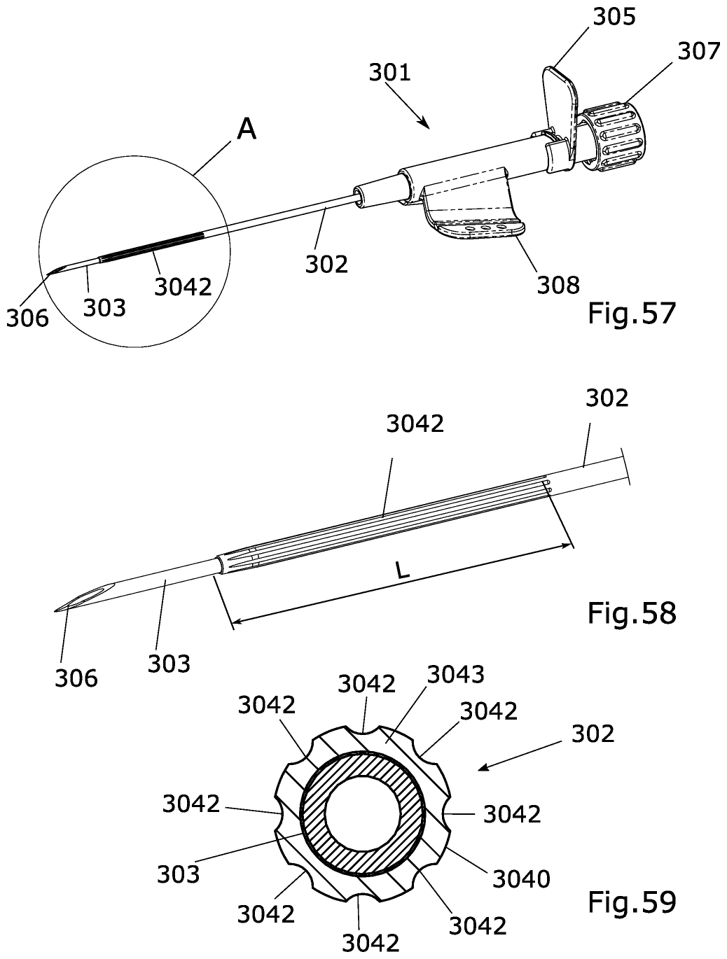

FIG. 57-59 show an indwelling venous cannula in a fourth embodiment,

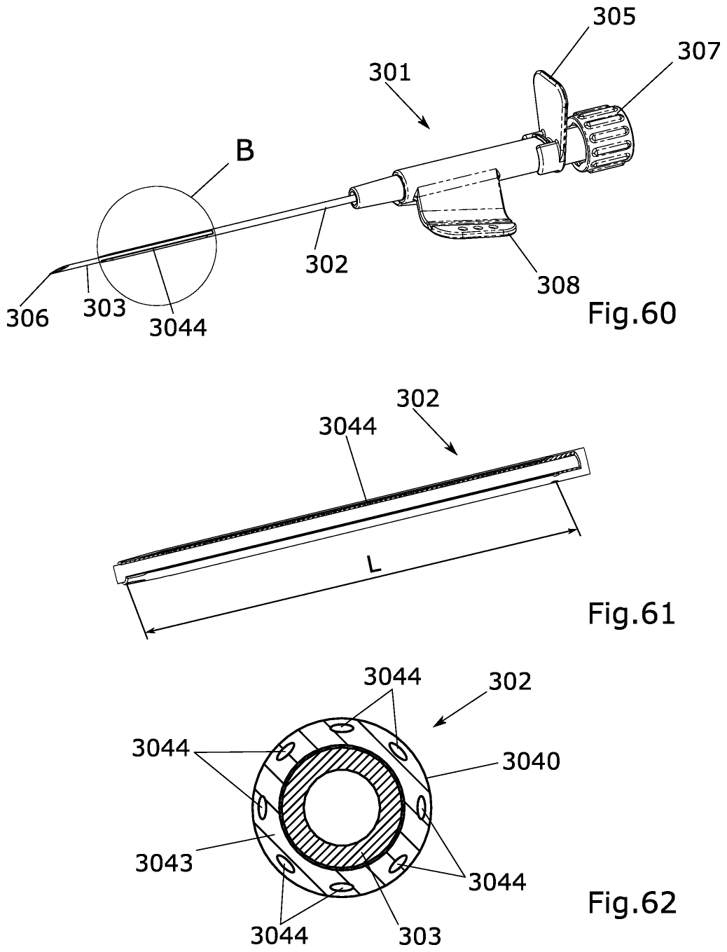

FIG. 60-62 show an indwelling venous cannula in a fifth embodiment,

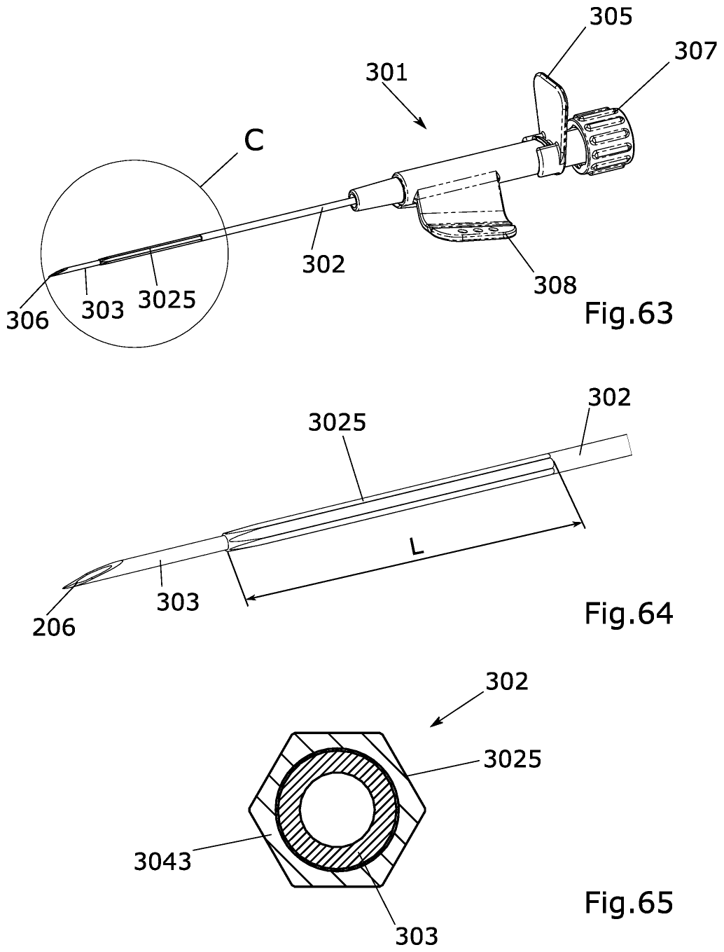

FIG. 63-65 show an indwelling venous cannula in a sixth embodiment,

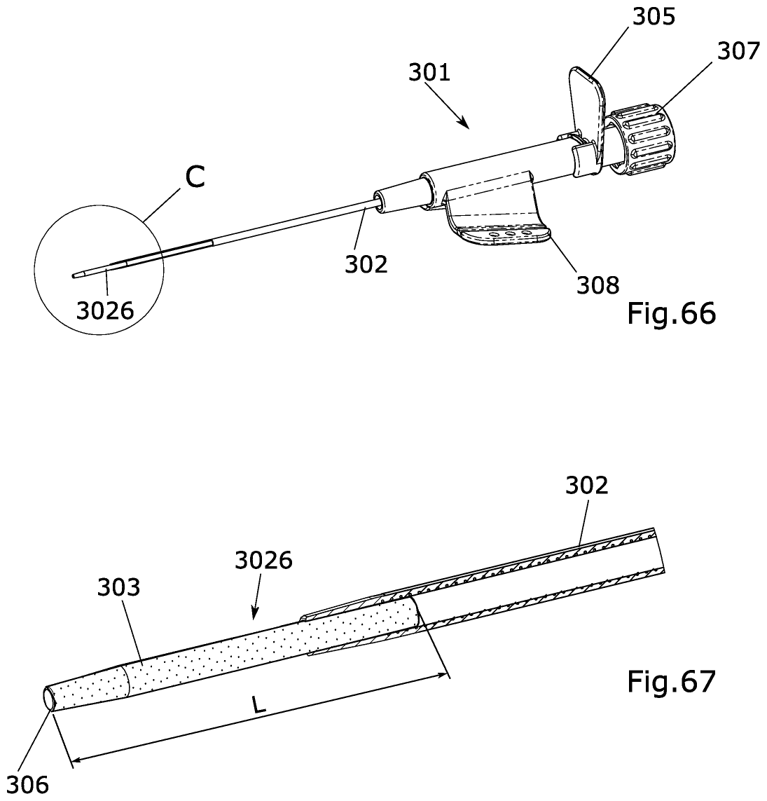

FIG. 66, 67 show an indwelling venous cannula in a seventh embodiment,

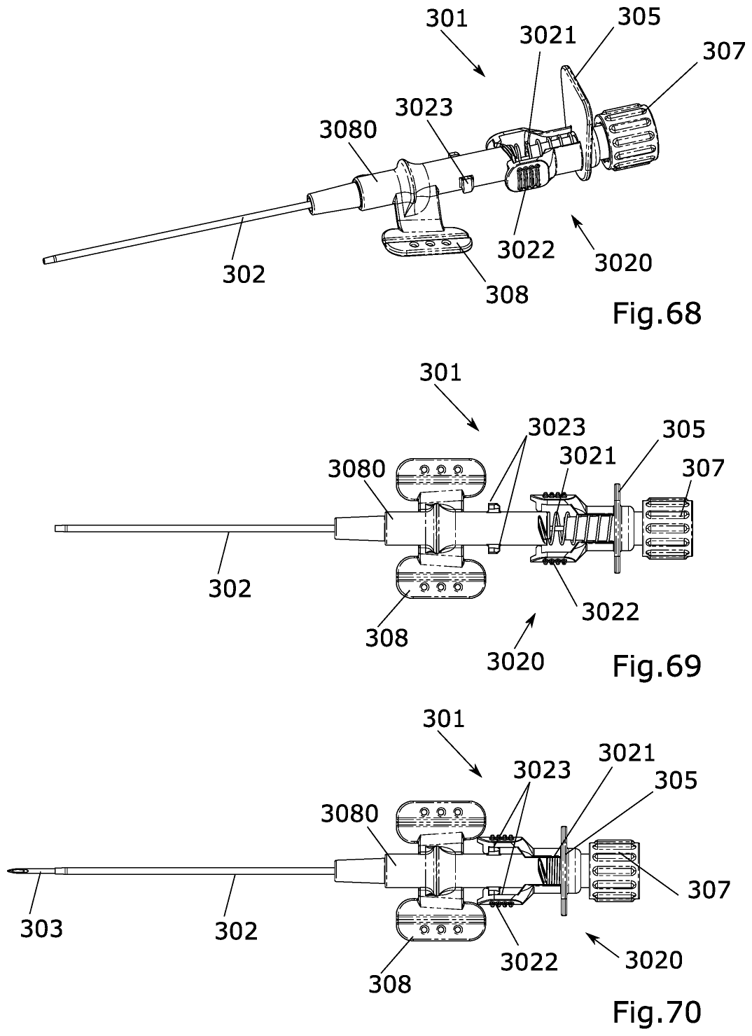

FIG. 68-70 show an indwelling venous cannula in an eighth embodiment,

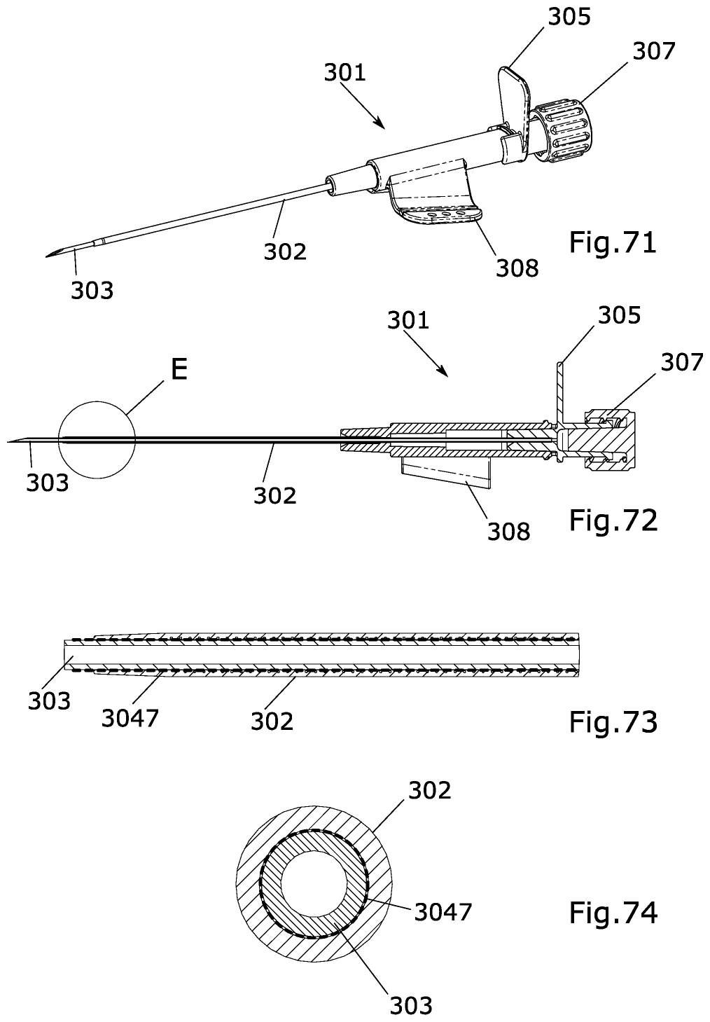

FIG. 71-74 show an indwelling venous cannula in a ninth embodiment,

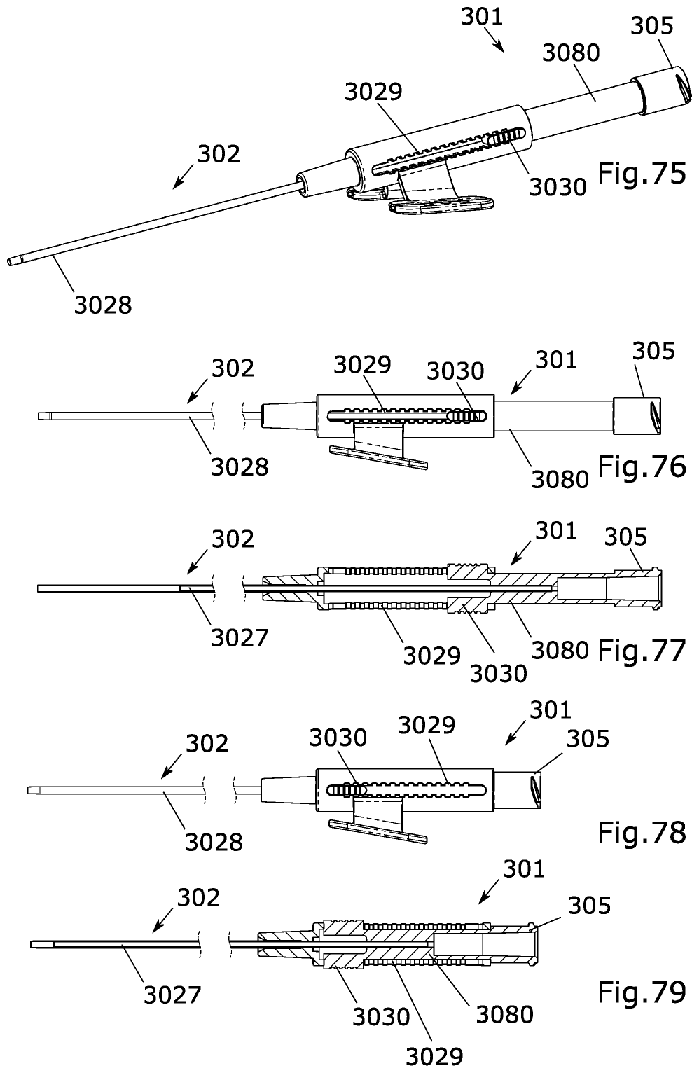

FIG. 75-79 show an indwelling venous cannula in a tenth embodiment,

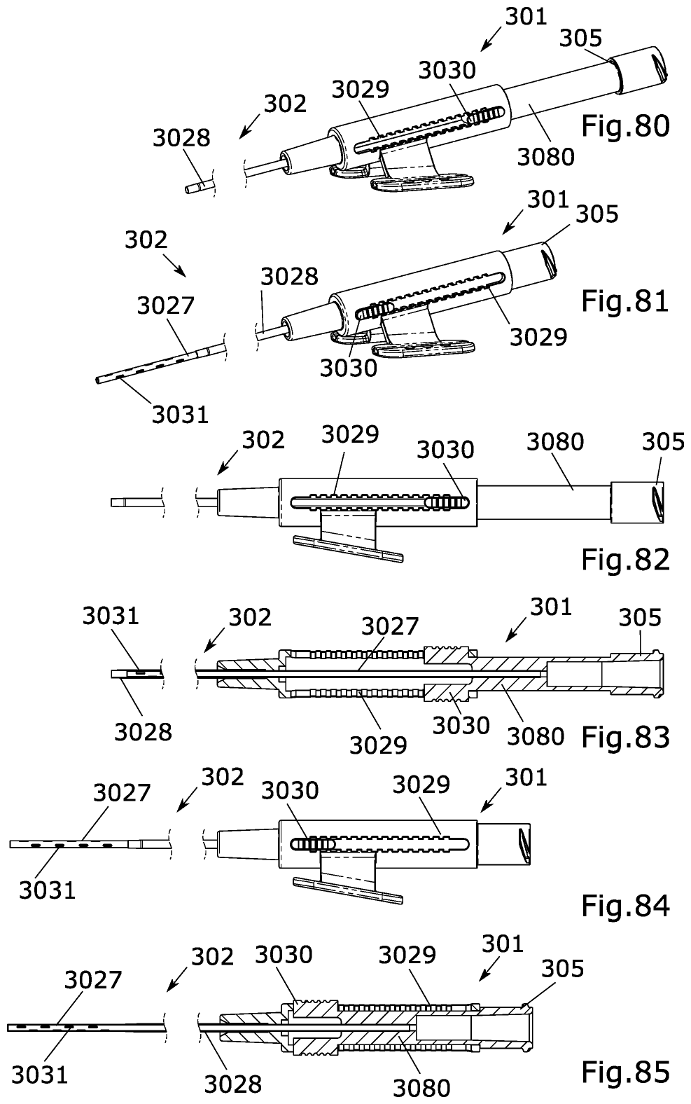

FIG. 80-85 show an indwelling venous cannula in an eleventh embodiment,

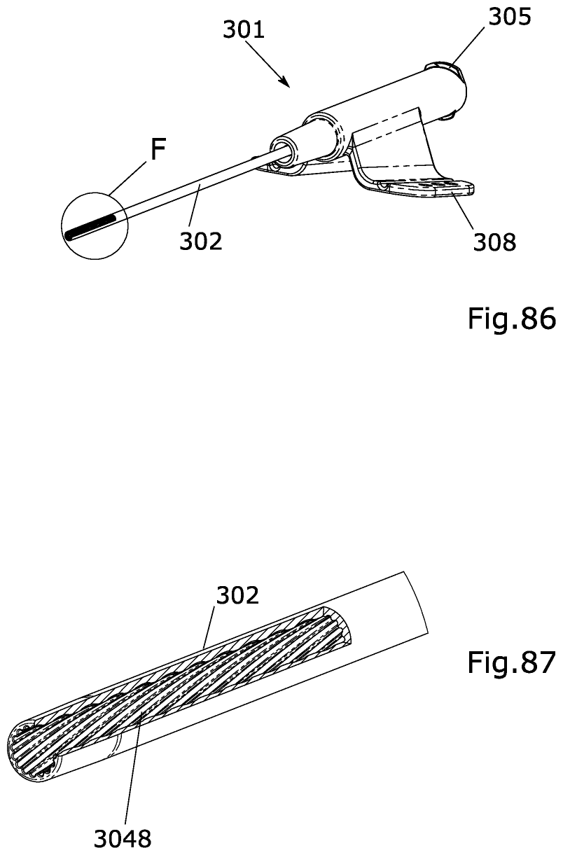

FIG. 86, 87 show an indwelling venous cannula in a twelfth embodiment,

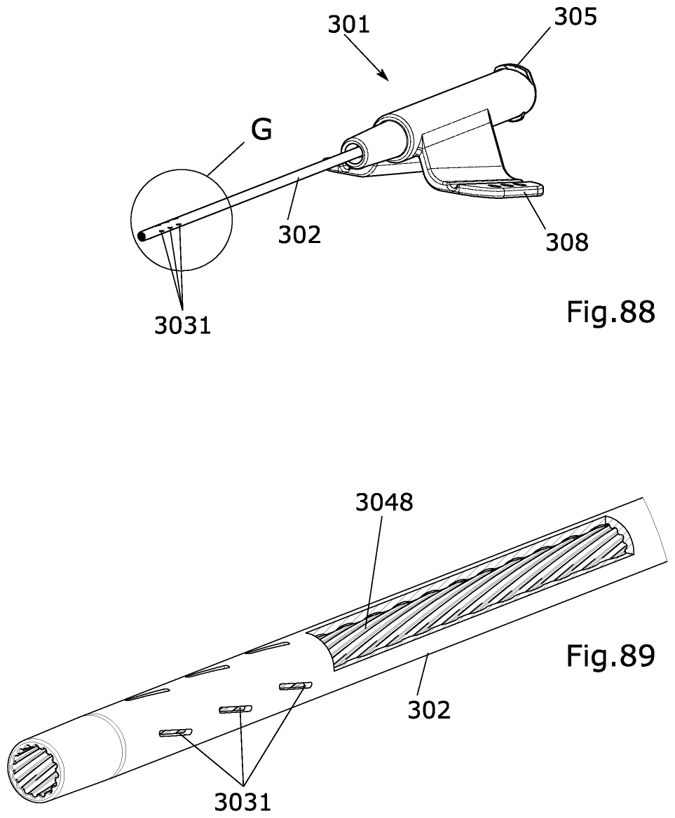

FIG. 88, 89 show an indwelling venous cannula in a thirteenth embodiment,

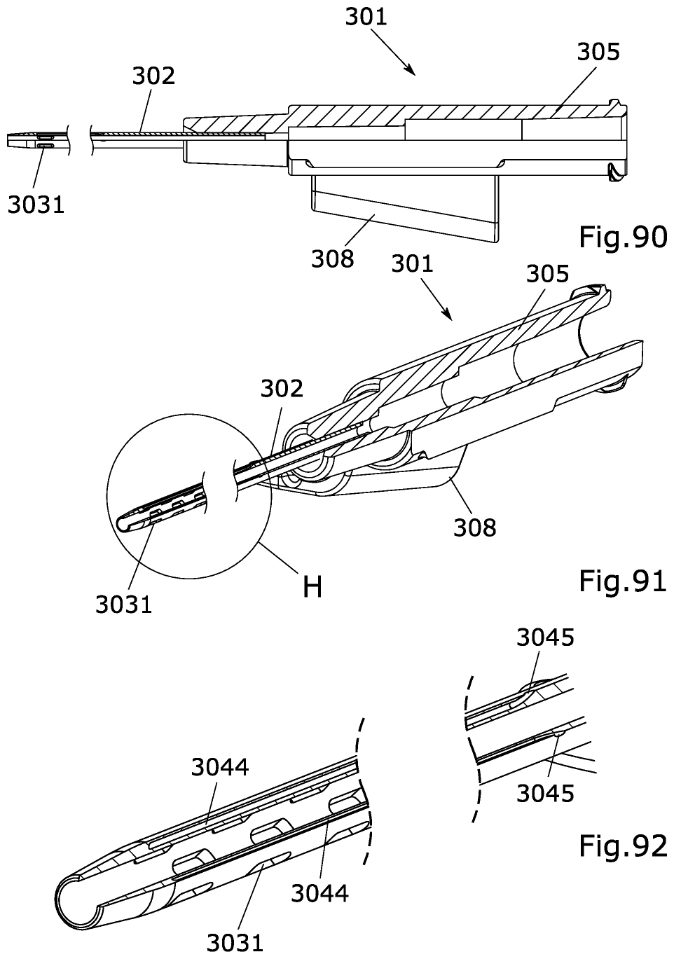

FIG. 90-92 show an indwelling venous cannula in a fourteenth embodiment.

FIG. 51 shows a schematic representation of an indwelling venous cannula 301 in a longitudinal section. The indwelling venous cannula 301 has a venous catheter 302; a puncture needle 303 can be guided longitudinally displaceably in the venous catheter 302.

The indwelling venous cannula 301 is in this case configured as a peripheral indwelling venous cannula 301. It is clear that the venous catheter 302 has been configured as a tightly wound spiral spring consisting of a piercing-resistant material, so that an undulating surface is created. The venous catheter 302 in this case consists of a piercing-resistant material over its entire length. The flexibility of the venous catheter 302 is ensured by the spiral structure. Such a version of the venous catheter 302 provides pricking and cutting protection which protects the venous catheter 302, for example, against piercing by a near-patient tip 306 of the puncture needle 303 during the application of the indwelling venous cannula 301. The risk of a damaged venous catheter 302 is thereby minimized. It can no longer be sheared by the near-patient tip 306 of the puncture needle 303 even by repeated displacement of the puncture needle 303 in relation to the venous catheter 302. Rejection due to damaged venous catheters may therefore be reduced significantly. Repeated use of the indwelling venous cannula 301 in the scope of a puncture process under permanent sterile conditions on a living being is also possible. This is important in particular when an incorrect puncture has initially been carried out, that is to say the blood vessel has been accidentally missed during the first puncture or it has at first not been possible to push the venous catheter 302 forward far enough into the blood vessel.

For improved aspiration, the venous catheter 302 is provided with a sealing coating 304. By the undulating surface, minimal openings that impede aspiration since, for example, air may be drawn undesirably through these openings, may be created. A sealing coating 304 may minimize or prevent the undesired aspiration of air. Advantageously, the sealing coating 304 is a PTFE coating which at the same time facilitates insertion of the venous catheter 302 into the punctured body part. By an additional dilatation element 3010 at the near-patient end of the venous catheter 302, uniform widening is achieved when the venous catheter 302 is pushed forward in the punctured body part.

The application of the indwelling venous cannula 301 on a living being may, for example, take place in the following steps:
1. puncturing a vein with the puncture needle 303
2. pushing the spiral venous catheter 302 forward with the sealing coating 304 beyond the near-patient tip 306 of the puncture needle 303 into the vein at the desired position
3. removing/withdrawing the puncture needle 303
4. sealing the spiral venous catheter 302 at a patient-remote end.

The indwelling venous cannula 301 has two holding elements 305. These holding elements 305 allow the user to operate the indwelling venous cannula 301 with one hand, in which case the second hand may for example be used to stabilize the body part to be punctured. The puncture needle 303 is configured as a hollow needle. After puncture has been carried out by the near-patient tip 306 of the puncture needle 303, the user can immediately identify whether the vein has been punctured correctly by the hollow puncture needle 303 being filled with venous blood and entering the chamber 307, so that the blood can be perceived directly by the user.

After puncture has been carried out, the venous catheter 302 may be pushed into the punctured body part and at the same time the puncture needle 303 together with the chamber 307 may be withdrawn from the components of the indwelling venous cannula 301 which remain in the body part. A securing mechanism may be configured so that the near-patient tip 306 of the puncture needle 303 is shielded after withdrawal from the indwelling venous cannula 301 and therefore protects the user as well as the living being against possible pricking injuries.

The venous catheter 302 may be held fixed on the living being in its end position in the punctured body part by means of fastening elements 308. The fixing may in this case be carried out by a self-adhesive dressing, which fixes the indwelling venous cannula 301 by means of the fastening elements 308 on the living being. The described fastening elements 308, which may for example be configured as wings, are optional elements of the indwelling venous cannula 301.

The puncture needle may in this case run substantially centrally between the holding elements 305 and/or the fastening elements 308.

It is clear that the functions of the holding element 305 and of the fastening element 308 may be combined in one element. This allows simple production of the indwelling venous cannula 301, while at the same time the construction of the indwelling venous cannula 301 is kept simple for the user.

By means of an attachment element 309, an aspiration element, for example a syringe, may be attached. The attachment element 309 may in this case be configured as a valve which allows simple medication delivery or aspiration of blood. The valve in this case prevents retrograde flow of liquids, for example blood, out of the attachment element 309 in the untouched state. The valve also prevents ingress of air from the outside into the attachment element 309 in the untouched state. The attachment element 309 may also contain a filter which prevents ingress of coarse particles, bacteria and air into the interior of the attachment element 309 and therefore into the interior of the indwelling venous cannula.

An aspiration element, for example a syringe, may be attached to the chamber 307. The indwelling venous cannula may thereby be introduced into a vein under constant aspiration with the syringe. The puncture outcome may therefore be established directly and very accurately. The chamber 307 may in this case also be formed as a further valve which allows the flow of fluids in only one defined direction. The chamber 307 may alternatively or additionally also be configured in such a way that it prevents ingress of air, or transmits air and other gases and vapors only in one defined direction. The chamber 307 may, for example, be configured in the manner of the attachment element 309.

The chamber 307 and the attachment element 309 may be covered by a protective cap, so that undesired contaminations do not occur when the chamber 307 and the attachment element 309 are not being used. The protective cap may in this case be connected respectively to the chamber 307 and/or to the attachment element 309 by means of a strap.

It is also conceivable that the flow of fluid in the indwelling venous cannula is interrupted or slowed by depressing a protective cap, which may be connected to the attachment element 309 or to the chamber 307 by means of a strap.

The venous catheter 302 has recesses 3011 arranged distributed over the circumference at the near-patient end. By the recesses 3011, it is possible to achieve homogeneous delivery, for example of a medication, into the living being. Undesired locally highly concentrated delivery of the medication into the living being is therefore avoided. By the arrangement of a plurality of recesses 3011, the flow rates of the applied infusion solutions and medications may also be increased. Aspiration of liquids, for example blood sampling, through the installed venous catheter or through the installed indwelling venous cannula from the living being may thereby also be facilitated. With a corresponding construction of other components of the indwelling venous cannula, a desired retrograde spontaneous exit of fluids, vapors and/or gases from the indwelling venous cannula may thereby also be possible, when it is intended to be used for example to drain fluids, vapors and/or gases, for example in the scope of puncturing the pleural space, other cavities or the interstitial body spaces described above.

As shown by FIGS. 52 and 53, the venous catheter 302 has a wall 3043. The wall has an inner side 3041 and an outer side 3040. The venous catheter 302, or the wall 3043, may have a circular or elliptical contour on the outer side 3040 or, as represented in FIG. 52, a polygonal contour, for example a hexagonal contour. In addition, flow channels in the form of indentations 3042 may be formed or introduced into the outer side 3040 of the wall 3043. The indentations 3042 may, for example, be configured as longitudinal grooves and extend over a more or less large longitudinal extent L1, L2, L3 along the venous catheter 302.

FIG. 54 shows an embodiment of the venous catheter 302 in which flow channels running in the longitudinal direction are present in the form of hollow cavities 3044 running inside the wall 3043. The hollow channels 3044 may in this case respectively have an entry opening 3045 for the fluid to flow in on the outer side 3040, as shown in FIGS. 55 and 56. The exit opening 3046, at which the fluid flowing through the hollow channel 3044 can flow back out of the respective hollow channel 3044, may selectively likewise be on the outer side 3040, as shown by FIG. 55, or on the inner side 3041 of the wall 3043, i.e. toward the inner lumen of the venous catheter 302.

The flow channels need not run exactly in the longitudinal direction, or parallel to the longitudinal direction, of the venous catheter 302, and they may also run wholly or in sections obliquely thereto, for example in a spiral shape.

FIGS. 57 to 59 show a further embodiment of an indwelling venous cannula 301, in which the venous catheter 302 has flow channels running in the longitudinal direction in the form of indentations 3042 on the outer side 3040 of the wall 3043. FIG. 57 shows the entire indwelling venous cannula 301 in a perspective view. The region A marked in FIG. 57 is represented as an enlarged detail in FIG. 58. FIG. 59 shows a cross-sectional view through the indwelling venous cannula 301 in the region of the indentations 3042. As may be seen, the venous catheter 302 has a circular contour on the outer side. The indentations 3042 extend over a length L in the longitudinal direction of the venous catheter 302.

FIGS. 60 to 62 show a further embodiment of an indwelling venous cannula 301, in which the venous catheter 302 has flow channels running in the longitudinal direction in the form of hollow channels 3044 running inside the wall 3043. FIG. 60 in this case shows the entire indwelling venous cannula 301 in a perspective view, FIG. 61 shows the region B marked in FIG. 60 in an enlarged representation and FIG. 62 shows a cross-sectional view through the venous catheter 301 in the region of the hollow channels 3044. It may be seen that the hollow channels 3044 need not necessarily have a circular cross-sectional shape, but may for example have a flattened cross-sectional shape, for example an elliptical contour or a curved contour following the arcuate shape of the wall 3043.

FIGS. 63 to 65 show an embodiment of an indwelling venous cannula 301 in which the venous catheter 302 has a polygonal outer contour 3025 on the outer side over a particular limited length section. FIG. 63 shows the entire indwelling venous cannula in a perspective view, FIG. 64 shows the region C marked in FIG. 63 in an enlarged detail representation and FIG. 65 shows a cross-sectional view through the region of the polygonal outer contour 3025. It may be seen that the venous catheter 302 may have the polygonal outer contour 3025 over a length section limited to the length L and in other regards may be configured as a conventional venous catheter, for example with a circular outer contour. For example, the polygonal region 3025 may be arranged at the near-patient end of the venous catheter 302. A hexagonal outer contour is represented by way of example, although other polygonal configurations are also possible.

FIGS. 66 and 67 show an embodiment of an indwelling venous cannula 301 in which the puncture needle 303 is configured bluntly at the tip 306 and at least a part of the puncture needle 303 consists of a bioresorbable material 3026. FIG. 66 shows the entire indwelling venous cannula 301 in a perspective view and FIG. 67 shows the region D marked in FIG. 66 in an enlarged detail representation. As may be seen, the puncture needle 303 is configured in the section near to the hollow body with a region consisting of bioresorbable material 3026, which is itself resorbed upon contact with the patient after a certain time. The puncture needle 303 is also configured comparatively bluntly at the tip 306. For example, the puncture needle 303 may consist of the bioresorbable material 3026 over a region of the length L. It is in addition possible that parts of the venous catheter 302, in particular in the region near to the hollow body, also consist of a bioresorbable material.

FIGS. 68 to 70 show an embodiment of an indwelling venous cannula 301 which has a spring retention mechanism 3020 for retaining the puncture needle 303 in the outer tunic (i.e. in the venous catheter 302). The spring retention mechanism 3020 is used as pricking protection in the initial or passive state of the indwelling venous cannula 301. In this state, the puncture needle 303 with its tip 306 is withdrawn fully into the venous catheter 302.

FIG. 68 shows the indwelling venous cannula 301 in a perspective view and FIGS. 69 and 70 show the indwelling venous cannula 301 in a plan view. In FIGS. 68 and 69, the spring retention mechanism is in the initial state, while in FIG. 70 it is in the activated state.

The spring retention mechanism 3020 may have a compression spring 3021 which generates, or at least in particular operating states can generate, a pressure force between a constituent part connected to the puncture needle 303, for example the chamber 307, and a constituent part connected to the venous catheter 302, for example a base body 3080, on which, for example, the holding elements 308 are fastened. The spring retention mechanism 3020 also has first latching elements 3022, which are coupled to the constituent part connected to the puncture needle 303, and second latching elements 3023, which are coupled to the base body 3080. If the indwelling venous cannula 301 is then converted into the activated state, namely by pushing the puncture needle 303 out of the venous catheter 302, as shown by FIG. 57, which may be carried out by applying a pressure force on the holding element 305, the compression spring 3021 is compressed. Beyond a particular setting, the first latching elements 3022 latch on the second latching elements 3023. The activated state of the indwelling venous cannula 301 is thereby adopted and maintained.

In order to release the latching, the first latching elements 3022 may simply be pressed on manually, i.e. they are compressed in the direction of the base body 3080. The latching may thereby be released, for example, by the second latching elements 3023 having a T-shaped profile. By the compression of the first latching elements 3022, they may then slide past the second latching elements 3023 while being assisted by the compression spring 3021. After release of the latching between the first latching elements 3022 and the second latching elements 3023, the puncture needle 303 is automatically withdrawn back into the venous catheter 302 by the force of the compression spring 3021.

FIGS. 71 to 74 show an embodiment of an indwelling venous cannula 301 in which a material 3047 having increased slideability is present on the outer side of the puncture needle 303 and/or the inner side of the outer tunic 302 over at least one longitudinal section. FIG. 71 shows the indwelling venous cannula 301 in a perspective view, FIG. 72 shows it in a longitudinal section, FIG. 73 shows the region E marked in FIG. 72 in an enlarged detail representation and FIG. 74 shows a cross section through the indwelling venous cannula 301. By the material 3074 effective between the outer side of the puncture needle and the inner side of the outer tunic 302, an increased slideability is provided between the puncture needle 303 and the outer tunic 302, particularly in the longitudinal displacement region of the puncture needle 303. The material 3047 may be present in the form of a friction-reducing auxiliary substance or in the form of individual roller bodies, in a similar way to a rolling bearing.

FIGS. 75, 76, 77, 78, 79 show an embodiment of an indwelling venous cannula 301 in which the outer tunic has two hollow bodies, namely an inner tube 3027 and an outer tube 3028. FIG. 75 shows the indwelling venous cannula in a perspective view, FIG. 76 shows it in a side view, FIG. 77 shows it in a longitudinal section, FIG. 78 shows it in a side view and FIG. 79 shows it in a longitudinal section. In FIGS. 75 to 77, the indwelling venous cannula 301 is in the retracted state, while in FIGS. 78 and 79 it is in the deployed state.

The inner tube 3027 is guided in the outer tube 3028 and is mounted longitudinally displaceably therein. The outer tube 3028 is firmly connected to the base body 3080. The inner tube 3027 is firmly connected to a displacement mechanism 3029. The displacement mechanism 3029 has a manual operating element 3030. The manual operating element 3030 can be moved forward and backward in a particular region of the base body 3080. The respective setting may, for example, be fixed by latching. If the manual operating element 3030 is for example moved from the setting represented in FIG. 77 into the setting represented in FIG. 79, the inner tube 3027 is moved with it and therefore pushed further into the outer tube 3028. In the retracted state, as represented for example in FIG. 77, the overlap between the inner tube 3027 and the outer tube 3028 is relatively small, while in the deployed state in FIG. 79 the overlap is substantially greater. In the retracted state, the rigidity of the outer tunic 302 is therefore less than in the deployed state, since in the deployed state the outer tube 3028 is additionally stiffened by the inner tube 3027 located therein. The stiffness of the outer tunic 302 may therefore be adjusted in particular stages or even continuously.

FIGS. 80 to 85 show an embodiment of an indwelling venous cannula 301 in which the outer tunic has two hollow bodies, namely an inner tube 3027 and an outer tube 3028. FIGS. 80 and 81 show perspective views, FIGS. 82 and 84 show side views and FIGS. 83 and 85 show longitudinal sections. In FIGS. 80, 82 and 83, the indwelling venous cannula is in the retracted state, while in FIGS. 81, 84 and 85 it is in the deployed state.

In contrast to the embodiment of FIGS. 75 to 79, in this embodiment the inner tube 3027 is configured to be substantially longer, in particular longer than the outer tube 3028. In the retracted state, the inner tube 3027 is fully or at least predominantly inside the outer tube 3028. In the deployed state, the inner tube 3027 protrudes by a particular extent from the outer tube 3028. For example, the application of the indwelling venous cannula on the patient may be carried out in the retracted state. During subsequent operation of the indwelling venous cannula on the patient, it is then converted into the deployed state. By means of the part of the inner tube 3027 protruding from the outer tube 3028, for example, medications may then be delivered or blood may be taken. The inner tube 3027 may in this case have the aforementioned lateral passage openings (side holes) at least in the region which may protrude from the outer tube 3028.

FIGS. 86 and 87 show an embodiment of an indwelling venous cannula 301 in which spiral or undulating structures 3048, for example spirally circumferential flow channels running predominantly in the longitudinal direction in the form of grooves, are formed on the inner side of the venous catheter 2. In general, the wall 3043 of the venous catheter 302 may have a plurality of spiral or undulating structures 3048, for example the structure 3048 which may be seen in FIG. 87 directed toward the inner side 3041, and alternatively or additionally spiral or undulating structures directed toward the outer side 3040. For example, the slideability of the puncture needle 303 in the venous catheter 302 may thereby be improved. Additional flow channels are furthermore provided. In this case, FIG. 86 shows the indwelling venous cannula 301 in a perspective representation and FIG. 87 shows the region F marked in FIG. 86 in an enlarged detail representation. By the spiral configuration of the structures 3048, a swirling effect of liquids guided through the outer tunic 302 may be achieved. For example, an infusion liquid may be swirled and thereby delivered to the patient while being distributed better.

FIGS. 88 and 89 show an embodiment of an indwelling venous cannula 301 in which spiral or undulating structures 3048, for example spirally circumferential flow channels running predominantly in the longitudinal direction in the form of grooves, are formed on the inner side of the venous catheter 302, in a similar way as in the previously described embodiment. In this case, FIG. 88 shows the indwelling venous cannula 301 in a perspective representation and FIG. 89 shows the region G marked in FIG. 88 in an enlarged detail representation. Additionally, the venous catheter 302 has a plurality of lateral passage openings 3031, through which liquids can flow, in the near-patient region.

FIGS. 90 to 92 show an embodiment of an indwelling venous cannula 301 in which the venous catheter 302 has a plurality of lateral passage openings 3031, through which liquids can flow, in the near-patient region, in a similar way as in the previously described embodiment. In this case, FIG. 90 shows the indwelling venous cannula 301 in a longitudinal section representation, FIG. 91 shows the indwelling venous cannula 301 in a perspective representation and FIG. 92 shows the region H marked in FIG. 91 in an enlarged detail representation. Additionally, the venous catheter 302 has a plurality of hollow channels 3044 running in the longitudinal direction inside the wall 3043 in the near-patient region. The hollow channels 3044 may in this case respectively have an entry opening 3045 but are configured without the exit opening 3046 described above, i.e. they are configured to be closed on at least one side. Advantageously, the entry opening 3045 is located in the region of the venous catheter 302 not inserted into the patient, i.e. away from the near-patient region. The hollow channels 3044 then extend into the near-patient region. These hollow channels 3044 may, for example, be filled with air or another medium that can be identified in an imaging examination. If the hollow channels are filled with air, for example, the visibility of the near-patient end of the venous catheter 302 in an ultrasound examination is made possible.

The present representations are merely schematic representations which give a good overview of the components of the indwelling venous cannula according to the invention. The lengths and size proportions may, however, differ in reality.

The figures are to be understood as a possible exemplary embodiment. Other forms of the teaching according to the invention may furthermore be envisioned. Furthermore, the configurations of the exemplary embodiment are not inseparably linked with one another, so that for example the implementation of the invention is not dependent on the specially described configurations of the exemplary embodiment. Thus, variability for example of the number, length or size of the individual elements may be envisioned at any time.

### LIST OF REFERENCE SIGNS

301 indwelling venous cannula
302 venous catheter/outer tunic
303 puncture needle
304 sealing coating
305 holding element
306 near-patient tip of the puncture needle
307 chamber
308 fastening element/holding element
309 attachment element
3010 dilatation element
3011 recess
3020 spring retention mechanism
3021 compression spring
3022 first latching element
3023 second latching element
3025 polygonal outer contour
3026 bioresorbable material
3027 inner tube
3028 outer tube
3029 displacement mechanism
3030 manual operating element
3031 lateral passage openings
3040 outer side
3041 inner side
3042 indentations
3043 wall
3044 hollow channels
3045 entry opening
3046 exit opening
3047 material with increased slideability
3048 spiral or undulating structure
3080 base body

1. An indwelling cannula for puncturing a hollow body by a puncture needle, comprising:
at least one catheter with a vasiform catheter tube, in which the puncture needle is guidable and longitudinally displaceably, wherein the at least one catheter is adapted to be pushed over at least a part of a length of the vasiform catheter tube through an opening created by the puncture needle through a tunic of the hollow body to be punctured after puncture of the tunic of the hollow body to be punctured, and wherein the at least one catheter is configured to remain through the tunic of the hollow body over a period of time.

2. The indwelling cannula as claimed in claim 1, further comprising a grip region which is grippable during manipulation of the indwelling cannula by the user.

3. The indwelling cannula as claimed in claim 1 further comprising a manually actuable forward feed mechanism by which a relative movement is generaratable between the vasiform catheter tube and the puncture needle as a result of manual actuation, by which a puncture tip, protruding from the vasiform catheter tube at a near-patient end of the puncture needle is receivable in the vasiform catheter tube.

4. The indwelling cannula as claimed in claim 3, wherein the forward feed mechanism is configured such that the vasiform catheter tube is pushable beyond the puncture tip of the puncture needle and/or the puncture tip of the puncture needle is withdrawable into the vasiform catheter tube.

5. The indwelling cannula as claimed in claim 3 wherein one or more operating elements of the manually actuable forward feed mechanism are arranged in the grip region and/or form parts of the grip region.

6. The indwelling cannula as claimed in claim 3 wherein feed forward mechanism is configured such that manual actuation of the forward feed mechanism limits relative movement between the vasiform catheter tube and the puncture needle to a maximum value.

7. The indwelling cannula as claimed in claim 6, wherein the maximum value of the relative movement is at least as great as a length of the puncture tip.

8. The indwelling cannula as claimed in claim 6 wherein the maximum value of the relative movement is less than two times a length of the puncture tip.

9. The indwelling cannula as claimed in claim 3 further comprising a lock for locking the forward feed mechanism in a manually actuated position in which the puncture tip is received in the vasiform catheter tube.

10. The indwelling cannula as claimed in claim 9, wherein in the manually actuated position of the forward feed mechanism in which the puncture tip is received in the vasiform catheter tube, at least one operating element of the forward feed mechanism is fixable by the lock and/or at least a part of a housing of the vasiform catheter is fixable on the needle device.

11. The indwelling cannula as claimed in claim 3 wherein at least one operating element of the forward feed mechanism is configured as a pivotably mounted lever with at least one lever arm.

12. The indwelling cannula as claimed in claim 3 wherein the forward feed mechanism is adapted to generate the relative movement between the vasiform catheter tube and the puncture needle by a manually applied actuation force and/or by a spring force.

13. The indwelling cannula as claimed in claim 3 wherein the forward feed mechanism comprises has at least two grip faces as operating elements, wherein on each of the at least two grip faces at least one finger of the user can be placed, wherein the at least two grip faces are being arranged turned away from one another.

14. The indwelling cannula as claimed in claim 13, wherein at least one of the at least two grip faces is arranged on a lever arm of a pivotably mounted lever of the forward feed mechanism.

15. The indwelling cannula as claimed in claim 14, wherein the lever is twistable about a rotation axis (D), wherein the at least one of the at least two grip faces arranged on the lever arm extends from a position which is further away than the rotation axis from the puncture needle to a position which is relatively closer to the puncture needle than the rotation axis in an unactuated state of the forward feed mechanism.

16. The indwelling cannula as claimed in claim 3 wherein the vasiform catheter tube has one or more markings by which the relative movement generated as a result of the manual actuation of the forward feed mechanism between the catheter tube and the puncture needle can be displayed.

17. The indwelling cannula as claimed in claim 3 wherein the forward feed mechanism has at least one intermediate setting which is haptically detectable by the user.

18. The indwelling cannula as claimed in claim 1 further comprising one or more reinforcing elements associated with the vasiform catheter tube by which a bending moment of resistance and/or a radial moment of resistance of the vasiform catheter tube is increasable relative to neighboring regions of the vasiform catheter tube which do not have a reinforcing element.

19. The indwelling cannula as claimed in claim 1 wherein at least a part of the vasiform catheter tube which is adapted to remain in the hollow body and has at least one spiral supporting structure over an entire length or over a predominant part of the length in which the puncture needle is guidable longitudinally displaceably.

20. The indwelling cannula as claimed in claim 1 further comprising a tube exit region in which the vasiform catheter tube projects from a housing of the indwelling cannula, wherein the indwelling cannula has a kink protection structure circumferentially enclosing the vasiform catheter tube fully or at least partially, by which a risk of the vasiform catheter tube kinking in a region of an exit site from the housing is reduced.

21. The indwelling cannula as claimed in claim 1 further comprising at least one finger stop device on a housing which is arranged on a tube exit region or near to the tube exit region.

22. The indwelling cannula as claimed in claim 1 further comprising one or more reinforcing elements associated with the vasiform catheter tube by which a bending moment of resistance and/or a radial moment of resistance of the vasiform catheter tube is increased relative to neighboring regions of the vasiform catheter tube.

23. The indwelling cannula as claimed in claim 1 wherein at least a part of the vasiform catheter tube is adapted to remain in the hollow body and has at least one spiral supporting structure over an entire length or over a predominant part of the length in which the puncture needle is guidable longitudinally displaceably.

24. The indwelling cannula as claimed in claim 23, wherein the spiral supporting structure has one or more spiral or undulating structures directed toward an inner side of the vasiform catheter tube and one or more spiral or undulating structures directed toward an outer side of the catheter tube.

25. The indwelling cannula as claimed in claim 23 wherein the at least spiral supporting structure has a smaller spacing of turns in front of one another in a region remote from the hollow body than in another region near to the hollow body.

26. The indwelling cannula as claimed in claim 1 wherein the vasiform catheter tube has lateral passage openings through which liquid can flow from an inner side to an outer side of the vasiform catheter tube and vice versa.

27. The indwelling cannula as claimed in claim 26, wherein one or more of the lateral passage openings are arranged in a region of the vasiform catheter tube not covered by a spiral supporting structure and/or between turns of the spiral supporting structure.

28. The indwelling cannula as claimed in claim 1 further comprising a tube exit region in which the vasiform catheter tube projects from a housing of the indwelling cannula; and,
a kink protection structure circumferentially enclosing the vasiform catheter tube fully or at least partially, by which a risk of the vasiform catheter tube kinking in the region of an exit site from the housing is reduced.

29. The indwelling cannula as claimed in claim 1 further comprising a housing of the vasiform catheter tube, wherein the housing has protecting projections for protecting the vasiform catheter tube, which extend from a tube exit region of the housing in a distal direction, and further comprising at least one guide slot running in a longitudinal direction and being present between the protecting projections, wherein the at least one guide slot at least a patient application side of the indwelling cannula, which is at least as wide as a diameter of the vasiform catheter tube so that the vasiform catheter tube can extend in a uniform arc through the at least one guide slot when the indwelling cannula is applied on the patient.

30. The indwelling cannula as claimed in claim 1 further comprising a grip region which is adapted such that the indwelling cannula is gripped in the grip region during manipulation by the user.

31. The indwelling cannula as claimed in claim 1 further comprising at least one finger stop device on a housing arranged on a tube exit region or near to the tube exit region.

32. The indwelling cannula as claimed in claim 1 further comprising at least one adhesive fastening element for self-adhesive fastening of the indwelling cannula on the patient.

33. The indwelling cannula as claimed in claim 1 further comprising at least one fixing wing or an arrangement of fixing wings coupled by a movement mechanism to a housing and by which a location of a least one fixing wing or of the arrangement of fixing wings in relation to the housing is readjustable.

34. A puncture device for puncturing a hollow body by a puncture needle, comprising: at least one vasiform outer tunic in which the puncture needle is guidable longitudinally displaceably, wherein the outer tunic is adapted to be pushable over at least a part of a length through an opening created by the puncture needle through a tunic of the hollow body to be punctured after puncture of the tunic of the hollow body to be punctured has been carried out, wherein the puncture device is configured to remain in the hollow body over a period of time.

35. The puncture device as claimed in claim 34, wherein the outer tunic and/or another part of the puncture device has one or more flow channels running predominantly or fully in a longitudinal direction of the puncture device through which a fluid flowing through a punctured hollow body can flow along past the outer tunic and/or the other part of the puncture device.

36. The puncture device as claimed in claim 34 wherein an outer side of the outer tunic comprises barb-like structures which impede or prevent extraction of the puncture device when inserted into the hollow body.

37. The puncture device as claimed in claim 34 wherein the outer tunic comprises an arrangement of a multiplicity of elongate cavities that extend at least predominantly in a longitudinal direction of the outer tunic.

38. The puncture device as claimed in claim 34 further comprising a further vasiform structure arranged between the outer tunic and the puncture needle.

39. The puncture device as claimed in claim 34 wherein an outer side of the puncture needle and/or an inner side of the outer tunic comprises a surface layer formed from a material having an increased slideability with respect to other material of the puncture needle and/or of the outer tunic.

40. The puncture device as claimed in claim 34 further comprising a friction-reducing auxiliary substance on an outer side of the puncture needle and/or an inner side of the outer tunic.

41. The puncture device as claimed in claim 34 wherein at least a part of the outer tunic which is adapted to remain in the hollow body has a spirally wound internally hollow guide wire over an entire length or over a predominant part of the entire length in which the puncture needle is guidable longitudinally displaceably.

42. The puncture device as claimed in claim 34 wherein a section of the puncture needle near to the hollow body is configured bluntly and/or consists of a bioresorbable material and/or any region of the puncture needle comprises a bioresorbable material or is coated with the bioresorbable material and/or at least a part of the outer tunic comprises a bioresorbable material or is coated with the bioresorbable material.

43. The puncture device as claimed in claim 34 wherein the puncture needle has an outer screw thread and/or the outer tunic has an inner screw thread.

44. The puncture device as claimed in claim 34 further comprising a guide wire which can be guided through the outer tunic or through the puncture needle, wherein the guide wire is formed partially, predominantly or fully from at least one bioresorbable material.

45. The puncture device as claimed in claim 34 wherein the puncture needle is twistable in relation to the outer tunic about a longitudinal axis, and wherein the puncture device further comprises holding elements by which the twistability of the puncture needle is restricted or negated at least in particular longitudinal displacement positions of the puncture needle in relation to the outer tunic.

46. The puncture device as claimed in claim 45, further comprising at least one restoring spring, wherein the puncture needle is held by a spring force of the at least one restoring spring in a longitudinal displacement position in which the twistability of the puncture needle is restricted or negated.

47. The puncture device as claimed in claim 34 wherein at least one constituent part of the puncture device has at least one marker that can be detected by an imaging examination apparatus.

48. The puncture device as claimed in claim 34 wherein the outer tunic has a fluid-permeable lattice-, network- or pore-like structure with fine openings which can be closed by blood constituents flowing through during operation of the puncture device.

49. The puncture device as claimed in claim 34 wherein at least parts of the puncture device have electrically insulating elements and/or electrically conductive elements, wherein the electrically conductive element provide one or more electrically conductive connections of one part of the puncture device to another part of the puncture device.

50. The puncture device as claimed in claim 34 wherein the outer tunic has a greater outer diameter in a region relatively nearer to the hollow body than in another region relatively remote from the hollow body.

51. The puncture device as claimed in claim 34 wherein at least one region of the outer tunic has a material which is swellable by contact with a fluid.

52. The puncture device as claimed in claim 34 further comprising an automatic blocking mechanism for preventing the puncture needle from being pushed forward toward an end near to the hollow body when the puncture needle has been withdrawn beyond a predetermined extent from the end near to the hollow body.

53. The puncture device as claimed in claim 34 wherein the outer tunic is configured in multiple layers with one or more removable layers.

54. The puncture device as claimed in claim 34 wherein a wall of the outer tunic has one spiral or undulating structure directed toward an inner side of the outer tunic and one spiral or undulating structure directed toward an outer side of the outer tunic.

55. The puncture device as claimed in claim 34 wherein the outer tunic has one or more reinforcing elements in one or more regions which are exposed to increased mechanical load.

56. The puncture device as claimed in claim 34 wherein the puncture device has at least one control element for adjusting a flexibility of at least one region of the outer tunic.

57. The puncture device as claimed in claim 34 further comprising an applicator at an end near to the hollow body for the application of a substance in a region of the entry site of the puncture needle on the hollow body.

58. The puncture device as claimed in claim 34 further comprising a mechanism for converting the outer tunic into a spiral configuration by a relative movement in relation to the puncture needle.

59. The puncture device as claimed in claim 34 wherein the outer tunic is configured with multiple lumens, wherein two or more lumens of the multiple lumens are connectable to one another via one or more openings.

60. The puncture device as claimed in claim 34 further comprising an automatic forward feed mechanism by which an automatic forward feed of the outer tunic into the punctured hollow body through the opening generated by means of the puncture needle is generated.

61. The puncture device as claimed in claim 34 further comprising a contamination protection device for protecting an end of the puncture device remote from the hollow body against contaminations.

62. The puncture device as claimed in claim 34 further comprising a spring retention mechanism for retaining the puncture needle in the outer tunic.

63. The puncture device as claimed in claim 34 wherein the puncture needle has two hollow bodies which are mounted longitudinally displaceably in relation to one another.

64. The puncture device as claimed in claim 34 wherein the outer tunic has two hollow bodies which are mounted longitudinally displaceably in relation to one another.

65. The puncture device as claimed in claim 34 wherein the outer tunic has lateral passage openings through which liquid can flow from an inner side to an outer side of the outer tunic and vice versa.
